SEQUENCE LISTING (31 sequences)

SEQ ID NO: 1 - protein, 261 aa, Artificial Sequence
  KLK2
MWDLVLSIALSVGCTGAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGGDSGGPLVCNGVLQGITSWGPEPCALPEKPAVYTKVVHYRKWIKDTIAANP

SEQ ID NO: 2 - protein, 405 aa, Artificial Sequence
  KLK2 fusion
MWDLVLSIALSVGCTGAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGGDSGGPLVCNGVLQGITSWGPEPCALPEKPAVYTKVVHYRKWIKDTIAANPGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 3 - RNA, 783 nt, Artificial Sequence
  KLK2 CDS
augugggaccugguucucuccaucgccuugucuguggggugcacuggugccgugccccucauccagucucggauugugggaggcugggagugugagaagcauucccaacccuggcagguggcuguguacagucauggaugggcacacuguggggguguccuggugcacccccagugggugcucacagcugcccauugccuaaagaagaauagccaggucuggcugggucggcacaaccuguuugagccugaagacacaggccagagggucccugucagccacagcuucccacacccgcucuacaauaugagccuucugaagcaucaaagccuuagaccagaugaagacuccagccaugaccucaugcugcuccgccugucagagccugccaagaucacagauguugugaagguccugggccugcccacccaggagccagcacuggggaccaccugcuacgccucaggcuggggcagcaucgaaccagaggaguucuugcgccccaggagucuucagugugugagccuccaucuccuguccaaugacaugugugcuagagcuuacucugagaaggugacagaguucauguugugugcugggcucuggacaggugguaaagacacuugugggggugauucuggggguccacuugucuguaauggugugcuucaagguaucacaucauggggcccugagccaugugcccugccugaaaagccugcuguguacaccaagguggugcauuaccggaaguggaucaaggacaccaucgcagccaacccc

SEQ ID NO: 4 - RNA, 1700 nt, Artificial Sequence
  KLK2 RNA
gggcgaacuaguauucuucugguccccacagacucagagagaacccgccaccaugugggaccugguucucuccaucgccuugucuguggggugcacuggugccgugccccucauccagucucggauugugggaggcugggagugugagaagcauucccaacccuggcagguggcuguguacagucauggaugggcacacuguggggguguccuggugcacccccagugggugcucacagcugcccauugccuaaagaagaauagccaggucuggcugggucggcacaaccuguuugagccugaagacacaggccagagggucccugucagccacagcuucccacacccgcucuacaauaugagccuucugaagcaucaaagccuuagaccagaugaagacuccagccaugaccucaugcugcuccgccugucagagccugccaagaucacagauguugugaagguccugggccugcccacccaggagccagcacuggggaccaccugcuacgccucaggcuggggcagcaucgaaccagaggaguucuugcgccccaggagucuucagugugugagccuccaucuccuguccaaugacaugugugcuagagcuuacucugagaaggugacagaguucauguugugugcugggcucuggacaggugguaaagacacuugugggggugauucuggggguccacuugucuguaauggugugcuucaagguaucacaucauggggcccugagccaugugcccugccugaaaagccugcuguguacaccaagguggugcauuaccggaaguggaucaaggacaccaucgcagccaaccccggaggauccggugguggcggcagcggcggcaagaagcaguacaucaaggccaacagcaaguucaucggcaucaccgagcugaagaagcugggagggggcaaacggggaggcggcaaaaagaugaccaacagcguggacgacgcccugaucaacagcaccaagaucuacagcuacuuccccagcgugaucagcaaagugaaccagggcgcucagggcaagaaacugggcucuagcggagggggaggcucuccuggcgggggaucuagcaucgugggaauuguggcaggacuggcagugcuggccgugguggugaucggagccgugguggcuaccgugaugugcagacggaaguccagcggaggcaagggcggcagcuacagccaggccgccagcucugauagcgcccagggcagcgacgugucacugacagccuaguaacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcuaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 5 - protein, 261 aa, Artificial Sequence
  PSA
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 6 - protein, 405 aa, Artificial Sequence
  PSA fusion
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANPGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 7 - RNA, 783 nt, Artificial Sequence
  PSA CDS
augugggucccgguugucuuccucacccuguccgugacguggauuggugcugcaccccucauccugucucggauugugggaggcugggagugcgagaagcauucccaacccuggcaggugcuuguggccucucguggcagggcagucugcggcgguguucuggugcacccccaguggguccucacagcugcccacugcaucaggaacaaaagcgugaucuugcugggucggcacagccuguuucauccugaagacacaggccagguauuucaggucagccacagcuucccacacccgcucuacgauaugagccuccugaagaaucgauuccucaggccaggugaugacuccagccacgaccucaugcugcuccgccugucagagccugccgagcucacggaugcugugaaggucauggaccugcccacccaggagccagcacuggggaccaccugcuacgccucaggcuggggcagcauugaaccagaggaguucuugaccccaaagaaacuucaguguguggaccuccauguuauuuccaaugacgugugugcgcaaguucacccucagaaggugaccaaguucaugcugugugcuggacgcuggacagggggcaaaagcaccugcucgggugauucugggggcccacuugucuguaauggugugcuucaagguaucacgucauggggcagugaaccaugugcccugcccgaaaggccuucccuguacaccaagguggugcauuaccggaaguggaucaaggacaccaucguggccaacccc

SEQ ID NO: 8 - RNA, 1700 nt, Artificial Sequence
  PSA RNA
gggcgaacuaguauucuucugguccccacagacucagagagaacccgccaccaugugggucccgguugucuuccucacccuguccgugacguggauuggugcugcaccccucauccugucucggauugugggaggcugggagugcgagaagcauucccaacccuggcaggugcuuguggccucucguggcagggcagucugcggcgguguucuggugcacccccaguggguccucacagcugcccacugcaucaggaacaaaagcgugaucuugcugggucggcacagccuguuucauccugaagacacaggccagguauuucaggucagccacagcuucccacacccgcucuacgauaugagccuccugaagaaucgauuccucaggccaggugaugacuccagccacgaccucaugcugcuccgccugucagagccugccgagcucacggaugcugugaaggucauggaccugcccacccaggagccagcacuggggaccaccugcuacgccucaggcuggggcagcauugaaccagaggaguucuugaccccaaagaaacuucaguguguggaccuccauguuauuuccaaugacgugugugcgcaaguucacccucagaaggugaccaaguucaugcugugugcuggacgcuggacagggggcaaaagcaccugcucgggugauucugggggcccacuugucuguaauggugugcuucaagguaucacgucauggggcagugaaccaugugcccugcccgaaaggccuucccuguacaccaagguggugcauuaccggaaguggaucaaggacaccaucguggccaaccccggaggauccggugguggcggcagcggcggcaagaagcaguacaucaaggccaacagcaaguucaucggcaucaccgagcugaagaagcugggagggggcaaacggggaggcggcaaaaagaugaccaacagcguggacgacgcccugaucaacagcaccaagaucuacagcuacuuccccagcgugaucagcaaagugaaccagggcgcucagggcaagaaacugggcucuagcggagggggaggcucuccuggcgggggaucuagcaucgugggaauuguggcaggacuggcagugcuggccgugguggugaucggagccgugguggcuaccgugaugugcagacggaaguccagcggaggcaagggcggcagcuacagccaggccgccagcucugauagcgcccagggcagcgacgugucacugacagccuaguaacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcuaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 9 - protein, 418 aa, Artificial Sequence
  PAP
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQVLKVIFAVAFCLISAVLMVLLFIHIRRGLCWQRESYGNI

SEQ ID NO: 10 - protein, 562 aa, Artificial Sequence
  PAP fusion
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQVLKVIFAVAFCLISAVLMVLLFIHIRRGLCWQRESYGNIGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 11 - RNA, 1254 nt, Artificial Sequence
  PAP CDS
augagagcugcuccccugcugcuggcaagagcugcuucucugucucugggauuucuguuucugcuguuuuucuggcuggauagaucugugcuggcaaaagaacugaaauuugugacccugguguuuagacacggagacagaucucccauugauaccuuucccaccgaucccaucaaagaaucuucuuggccacagggauuuggacagcugacccagcugggaauggagcagcacuacgaacugggagaauacaucagaaaaagauacagaaaauuucugaaugaaucuuacaaacacgaacagguguacaucagaucuaccgauguggauagaacccugaugucugcaaugaccaaucuggcugcucuguuucccccagaaggagugagcaucuggaaucccauccugcuguggcagcccaucccugugcacaccgugccucugucugaagaucagcugcuguaccugccuuuuagaaauugcccaagauuucaggaacuugaaucugaaacccugaaaucugaagaauuucagaaaagacugcacccuuacaaagauuuuaucgcaacccugggaaaacugucuggacugcacggacaggaucucuuuggaauuuggucaaaaguguacgauccucuguacugugaaucugugcacaauuuuacccugcccucuugggcaaccgaagauaccaugaccaaacugagagaacugucugaacugucucugcugucucuguacggaauccacaaacagaaagaaaaaucaagacugcagggaggagugcuggugaaugaaauccugaaucacaugaaaagagcaacccagaucccaucuuacaagaagcugaucauguacucugcucacgauaccacagugucuggacugcagauggcucuggauguguacaauggacugcugccucccuacgcuucuugccaccugaccgaacuguacuuugaaaaaggagaauacuuuguggaaauguacuacagaaaugaaacccagcacgaaccuuacccccugaugcugccuggaugcucucccucuugcccucuggaaagauuugcugaacuggugggaccugugaucccucaggauugguccacagaaugcaugaccaccaauucucaccaggugcugaaagugaucuuugcuguggcuuuuugccugaucucugcugugcugauggugcugcuguuuauccacaucaggagaggacugugcuggcagagagaaucuuacggaaauauc

SEQ ID NO: 12 - RNA, 2171 nt, Artificial Sequence
  PAP RNA
gggcgaacuaguauucuucugguccccacagacucagagagaacccgccaccaugagagcugcuccccugcugcuggcaagagcugcuucucugucucugggauuucuguuucugcuguuuuucuggcuggauagaucugugcuggcaaaagaacugaaauuugugacccugguguuuagacacggagacagaucucccauugauaccuuucccaccgaucccaucaaagaaucuucuuggccacagggauuuggacagcugacccagcugggaauggagcagcacuacgaacugggagaauacaucagaaaaagauacagaaaauuucugaaugaaucuuacaaacacgaacagguguacaucagaucuaccgauguggauagaacccugaugucugcaaugaccaaucuggcugcucuguuucccccagaaggagugagcaucuggaaucccauccugcuguggcagcccaucccugugcacaccgugccucugucugaagaucagcugcuguaccugccuuuuagaaauugcccaagauuucaggaacuugaaucugaaacccugaaaucugaagaauuucagaaaagacugcacccuuacaaagauuuuaucgcaacccugggaaaacugucuggacugcacggacaggaucucuuuggaauuuggucaaaaguguacgauccucuguacugugaaucugugcacaauuuuacccugcccucuugggcaaccgaagauaccaugaccaaacugagagaacugucugaacugucucugcugucucuguacggaauccacaaacagaaagaaaaaucaagacugcagggaggagugcuggugaaugaaauccugaaucacaugaaaagagcaacccagaucccaucuuacaagaagcugaucauguacucugcucacgauaccacagugucuggacugcagauggcucuggauguguacaauggacugcugccucccuacgcuucuugccaccugaccgaacuguacuuugaaaaaggagaauacuuuguggaaauguacuacagaaaugaaacccagcacgaaccuuacccccugaugcugccuggaugcucucccucuugcccucuggaaagauuugcugaacuggugggaccugugaucccucaggauugguccacagaaugcaugaccaccaauucucaccaggugcugaaagugaucuuugcuguggcuuuuugccugaucucugcugugcugauggugcugcuguuuauccacaucaggagaggacugugcuggcagagagaaucuuacggaaauaucggaggauccggugguggcggcagcggcggcaagaagcaguacaucaaggccaacagcaaguucaucggcaucaccgagcugaagaagcugggagggggcaaacggggaggcggcaaaaagaugaccaacagcguggacgacgcccugaucaacagcaccaagaucuacagcuacuuccccagcgugaucagcaaagugaaccagggcgcucagggcaagaaacugggcucuagcggagggggaggcucuccuggcgggggaucuagcaucgugggaauuguggcaggacuggcagugcuggccgugguggugaucggagccgugguggcuaccgugaugugcagacggaaguccagcggaggcaagggcggcagcuacagccaggccgccagcucugauagcgcccagggcagcgacgugucacugacagccuaguaacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcuaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 13 - protein, 284 aa, Artificial Sequence
  HOXB13
MEPGNYATLDGAKDIEGLLGAGGGRNLVAHSPLTSHPAAPTLMPAVNYAPLDLPGSAEPPKQCHPCPGVPQGTSPAPVPYGYFGGGYYSCRVSRSSLKPCAQAATLAAYPAETPTAGEEYPSRPTEFAFYPGYPGTYQPMASYLDVSVVQTLGAPGEPRHDSLLPVDSYQSWALAGGWNSQMCCQGEQNPPGPFWKAAFADSSGQHPPDACAFRRGRKKRIPYSKGQLRELEREYAANKFITKDKRRKISAATSLSERQITIWFQNRRVKEKKVLAKVKNSATP

SEQ ID NO: 14 - protein, 464 aa, Artificial Sequence
  HOXB13 fusion
MRVMAPRTLILLLSGALALTETWAGSGGSGGGGSGGMEPGNYATLDGAKDIEGLLGAGGGRNLVAHSPLTSHPAAPTLMPAVNYAPLDLPGSAEPPKQCHPCPGVPQGTSPAPVPYGYFGGGYYSCRVSRSSLKPCAQAATLAAYPAETPTAGEEYPSRPTEFAFYPGYPGTYQPMASYLDVSVVQTLGAPGEPRHDSLLPVDSYQSWALAGGWNSQMCCQGEQNPPGPFWKAAFADSSGQHPPDACAFRRGRKKRIPYSKGQLRELEREYAANKFITKDKRRKISAATSLSERQITIWFQNRRVKEKKVLAKVKNSATPGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 15 - RNA, 852 nt, Artificial Sequence
  HOXB13 CDS
auggagcccggcaauuaugccaccuuggauggagccaaggauaucgaaggcuugcugggagcgggaggggggcggaaucuggucgcccacuccccucugaccagccacccagcggcgccuacgcugaugccugcugucaacuaugcccccuuggaucugccaggcucggcggagccgccaaagcaaugccacccaugcccuggggugccccaggggacguccccagcucccgugccuuaugguuacuuuggaggcggguacuacuccugccgagugucccggaguucgcugaaacccugugcccaggcagccacccuggccgcguaccccgcggagacucccacggccggggaggaguaccccagccgccccacugaguuugccuucuauccgggauauccgggaaccuaccagccuauggccaguuaccuggacgugucuguggugcagacucugggugcuccuggagaaccgcgacaugacucccuguugccuguggacaguuaccagucuugggcucucgcugguggcuggaacagccagauguguugccagggagaacagaacccaccaggucccuuuuggaaggcagcauuugcagacuccagcgggcagcacccuccugacgccugcgccuuucgucgcggccgcaagaaacgcauuccguacagcaaggggcaguugcgggagcuggagcgggaguaugcggcuaacaaguucaucaccaaggacaagaggcgcaagaucucggcagccaccagccucucggagcgccagauuaccaucugguuucagaaccgccgggucaaagagaagaagguucucgccaaggugaagaacagcgcuaccccu

SEQ ID NO: 16 - RNA, 1877 nt, Artificial Sequence
  HOXB13 RNA
gggcgaacuaguauucuucugguccccacagacucagagagaacccgccaccaugagagugauggcccccagaacccugauccugcugcugucuggcgcccuggcccugacagagacaugggccggaagcggcggcucuggaggaggcggcuccggaggcauggagcccggcaauuaugccaccuuggauggagccaaggauaucgaaggcuugcugggagcgggaggggggcggaaucuggucgcccacuccccucugaccagccacccagcggcgccuacgcugaugccugcugucaacuaugcccccuuggaucugccaggcucggcggagccgccaaagcaaugccacccaugcccuggggugccccaggggacguccccagcucccgugccuuaugguuacuuuggaggcggguacuacuccugccgagugucccggaguucgcugaaacccugugcccaggcagccacccuggccgcguaccccgcggagacucccacggccggggaggaguaccccagccgccccacugaguuugccuucuauccgggauauccgggaaccuaccagccuauggccaguuaccuggacgugucuguggugcagacucugggugcuccuggagaaccgcgacaugacucccuguugccuguggacaguuaccagucuugggcucucgcugguggcuggaacagccagauguguugccagggagaacagaacccaccaggucccuuuuggaaggcagcauuugcagacuccagcgggcagcacccuccugacgccugcgccuuucgucgcggccgcaagaaacgcauuccguacagcaaggggcaguugcgggagcuggagcgggaguaugcggcuaacaaguucaucaccaaggacaagaggcgcaagaucucggcagccaccagccucucggagcgccagauuaccaucugguuucagaaccgccgggucaaagagaagaagguucucgccaaggugaagaacagcgcuaccccuggaggauccggugguggcggcagcggcggcaagaagcaguacaucaaggccaacagcaaguucaucggcaucaccgagcugaagaagcugggagggggcaaacggggaggcggcaaaaagaugaccaacagcguggacgacgcccugaucaacagcaccaagaucuacagcuacuuccccagcgugaucagcaaagugaaccagggcgcucagggcaagaaacugggcucuagcggagggggaggcucuccuggcgggggaucuagcaucgugggaauuguggcaggacuggcagugcuggccgugguggugaucggagccgugguggcuaccgugaugugcagacggaaguccagcggaggcaagggcggcagcuacagccaggccgccagcucugauagcgcccagggcagcgacgugucacugacagccuaguaacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcuaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 17 - protein, 234 aa, Artificial Sequence
  NKX3-1
MLRVPEPRPGEAKAEGAAPPTPSKPLTSFLIQDILRDGAQRQGGRTSSQRQRDPEPEPEPEPEGGRSRAGAQNDQLSTGPRAAPEEAETLAETEPERHLGSYLLDSENTSGALPRLPQTPKQPQKRSRAAFSHTQVIELERKFSHQKYLSAPERAHLAKNLKLTETQVKIWFQNRRYKTKRKQLSSELGDLEKHSSLPALKEEAFSRASLVSVYNSYPYYPYLYCVGSWSPAFW

SEQ ID NO: 18 - protein, 414 aa, Artificial Sequence
  NKX3-1 fusion
MRVMAPRTLILLLSGALALTETWAGSGGSGGGGSGGMLRVPEPRPGEAKAEGAAPPTPSKPLTSFLIQDILRDGAQRQGGRTSSQRQRDPEPEPEPEPEGGRSRAGAQNDQLSTGPRAAPEEAETLAETEPERHLGSYLLDSENTSGALPRLPQTPKQPQKRSRAAFSHTQVIELERKFSHQKYLSAPERAHLAKNLKLTETQVKIWFQNRRYKTKRKQLSSELGDLEKHSSLPALKEEAFSRASLVSVYNSYPYYPYLYCVGSWSPAFWGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 19 - RNA, 702 nt, Artificial Sequence
  NKX3-1 CDS
augcugagagugcccgaaccuagaccuggcgaggcuaaagcugaaggcgcugcuccuccuacaccuagcaagccucugaccagcuuccugauccaagacauccugagagauggcgcccagagacaaggcggcagaacaagcucucagagacagagagaucccgagccugagccugaaccagaaccugaaggcggaagaucuagagccggcgcucagaacgaucagcugagcacaggaccuagagccgcuccugaggaagccgaaacacuggccgaaaccgagccagagagacaccugggcagcuaccugcuggacagcgagaauacuucuggcgcccugccuagacugccccagacaccuaaacagccucagaagcggagcagagccgccuuuagccacacacaagugaucgagcuggaacggaaguucagccaccagaaguaccugagcgccccugaaagagcccaccuggccaagaaucugaagcugaccgagacucaagugaagaucugguuccagaaccggcgguacaagaccaagcggaagcagcugucaagcgagcugggcgaccuggaaaagcacagcucucugcccgcucugaaagaggaagccuucuccagagccagccugguguccguguacaacagcuacccuuacuaccccuaccuguacugcgucggcucuuggagcccugccuuuugg

SEQ ID NO: 20 - RNA, 1727 nt, Artificial Sequence
  NKX3-1 RNA
gggcgaacuaguauucuucugguccccacagacucagagagaacccgccaccaugagagugauggcccccagaacccugauccugcugcugucuggcgcccuggcccugacagagacaugggccggaagcggcggcucuggaggaggcggcuccggaggcaugcugagagugcccgaaccuagaccuggcgaggcuaaagcugaaggcgcugcuccuccuacaccuagcaagccucugaccagcuuccugauccaagacauccugagagauggcgcccagagacaaggcggcagaacaagcucucagagacagagagaucccgagccugagccugaaccagaaccugaaggcggaagaucuagagccggcgcucagaacgaucagcugagcacaggaccuagagccgcuccugaggaagccgaaacacuggccgaaaccgagccagagagacaccugggcagcuaccugcuggacagcgagaauacuucuggcgcccugccuagacugccccagacaccuaaacagccucagaagcggagcagagccgccuuuagccacacacaagugaucgagcuggaacggaaguucagccaccagaaguaccugagcgccccugaaagagcccaccuggccaagaaucugaagcugaccgagacucaagugaagaucugguuccagaaccggcgguacaagaccaagcggaagcagcugucaagcgagcugggcgaccuggaaaagcacagcucucugcccgcucugaaagaggaagccuucuccagagccagccugguguccguguacaacagcuacccuuacuaccccuaccuguacugcgucggcucuuggagcccugccuuuuggggaggauccggugguggcggcagcggcggcaagaagcaguacaucaaggccaacagcaaguucaucggcaucaccgagcugaagaagcugggagggggcaaacggggaggcggcaaaaagaugaccaacagcguggacgacgcccugaucaacagcaccaagaucuacagcuacuuccccagcgugaucagcaaagugaaccagggcgcucagggcaagaaacugggcucuagcggagggggaggcucuccuggcgggggaucuagcaucgugggaauuguggcaggacuggcagugcuggccgugguggugaucggagccgugguggcuaccgugaugugcagacggaaguccagcggaggcaagggcggcagcuacagccaggccgccagcucugauagcgcccagggcagcgacgugucacugacagccuaguaacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcuaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 21 - RNA, 52 nt, Artificial Sequence
  5′ UTR
gggcgaacuaguauucuucugguccccacagacucagagagaacccgccacc

SEQ ID NO: 22 - protein, 26 aa, Artificial Sequence
  Secretory signal
MRVMAPRTLILLLSGALALTETWAGS

SEQ ID NO: 23 - RNA, 78 nt, Artificial Sequence
  Secretory signal
augagagugauggcccccagaacccugauccugcugcugucuggcgcccuggcccugacagagacaugggccggaagc

SEQ ID NO: 24 - protein, 55 aa, Artificial Sequence
  MITD
IVGIVAGLAVLAVVVIGAVVATVMCRRKSSGGKGGSYSQAASSDSAQGSDVSLTA

SEQ ID NO: 25 - RNA, 171 nt, Artificial Sequence
  MITD
aucgugggaauuguggcaggacuggcagugcuggccgugguggugaucggagccgugguggcuaccgugaugugcagacggaaguccagcggaggcaagggcggcagcuacagccaggccgccagcucugauagcgcccagggcagcgacgugucacugacagccuaguaa

SEQ ID NO: 26 - protein, 65 aa, Artificial Sequence
  P2P16
KKQYIKANSKFIGITELKKLGGGKRGGGKKMTNSVDDALINSTKIYSYFPSVISKVNQGAQGKKL

SEQ ID NO: 27 - RNA, 195 nt, Artificial Sequence
  P2P16
aagaagcaguacaucaaggccaacagcaaguucaucggcaucaccgagcugaagaagcugggagggggcaaacggggaggcggcaaaaagaugaccaacagcguggacgacgcccugaucaacagcaccaagaucuacagcuacuuccccagcgugaucagcaaagugaaccagggcgcucagggcaagaaacug

SEQ ID NO: 28 - RNA, 317 nt, Artificial Sequence
  3′ UTR
cucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcu

SEQ ID NO: 29 - RNA, 110 nt, Artificial Sequence
  A30L70
aaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 30 - protein, 15 aa, Artificial Sequence
  P2
QYIKANSKFIGITEL

SEQ ID NO: 31 - protein, 32 aa, Artificial Sequence
  P16
MTNSVDDALINSTKIYSYFPSVISKVNQGAQG

(19) United States
(12) Patent Application Publication
(10) Pub. No.: US 2023/0114808 A1
(43) Pub. Date: Apr. 13, 2023

(54) THERAPEUTIC RNA FOR PROSTATE CANCER

(71) Applicants: BioNTech SE, Mainz (DE); TRON-Translationale Onkologie an der Universitatsmedizin der Johannes Gutenberg-Universitat Gemein-N, Mainz (DE)

(72) Inventors: David WEBER, Mainz (DE); Carina WALTER, Mainz (DE); Diana BAREA ROLDAN, Mainz (DE); Ruprecht KUNER, Mainz (DE); Elif DIKEN, Mainz (DE); Martin SUCHAN, Mainz (DE); Stefania GANGI MAURICI, Mainz (DE); Ugur SAHIN, Mainz (DE)

(21) Appl. No.: 17/310,773

(22) PCT Filed: Mar. 11, 2020
(86) PCT No.: PCT/EP2020/056476

§ 371 Date: Aug. 23, 2021

(30) Foreign Application Priority Data
Mar. 12, 2019 (EP) PCT/EP2019/056185

(51) Int. Cl. A61K 39/00; A61P 35/00; A61P 37/04; A61K 45/06; A61K 39/395; A61K 39/39

(52) U.S. Cl. CPC A61K 39/00116; A61K 39/001194; A61K 39/001193; A61K 39/001152; A61P 35/00; A61P 37/04; A61K 45/06; A61K 39/3955; A61K 39/39; A61K 2039/53; A61K 2039/884; A61K 2039/55544; A61K 2039/70; A61K 2039/55555

(57) ABSTRACT

Disclosed herein are compositions, uses, and methods for treatment of prostate cancers. In one aspect, disclosed herein is a composition or medical preparation comprising at least one RNA, wherein the at least one RNA encodes the following amino acid sequences: (i) an amino acid sequence comprising Kallikrein-2 (KLK2), an immunogenic variant thereof, or an immunogenic fragment of the KLK2 or the immunogenic variant thereof; (ii) an amino acid sequence comprising Prostate Specific Antigen (PSA), an immunogenic variant thereof, or an immunogenic fragment of the PSA or the immunogenic variant thereof; (iii) an amino acid sequence comprising Prostatic Acid Phosphatase (PAP), an immunogenic variant thereof, or an immunogenic fragment of the PAP or the immunogenic variant thereof; (iv) an amino acid sequence comprising Homeobox B13 (HOXB13), an immunogenic variant thereof, or an immunogenic fragment of the HOXB13 or the immunogenic variant thereof; and (v) an amino acid sequence comprising NK3 Homeobox 1 (NKX3-1), an immunogenic variant thereof, or an immunogenic fragment of the NKX3-1 or the immunogenic variant thereof.

### CROSS-REFERENCE TO RELATED APPLICATIONS

This application is a National Stage Entry of International Application Number PCT/EP2020/056476, which was filed on Mar. 11, 2020 and claimed priority to International Application Number PCT/EP2019/056185, which was filed on Mar. 12, 2019. The contents of each of the aforementioned applications are incorporated herein by reference in their entireties.

This disclosure relates to the field of therapeutic RNA to treat prostate cancer. Prostate cancer is a serious disease that affects thousands of men each year who are middle-aged or older. About 60 percent of the cases occur in men older than age 65. The American Cancer Society (ACS) estimates that 174,650 American men will be newly diagnosed with this condition in 2019. According to the Urology Care Foundation, prostate cancer is the second-leading cause of cancer deaths for men in the United States.

Disclosed herein are compositions, uses, and methods for treatment of prostate cancers. Administration of therapeutic RNAs to a patient having prostate cancer disclosed herein can reduce tumor size, prolong time to progressive disease, and/or protect against metastasis and/or recurrence of the tumor and ultimately extend survival time.

### SUMMARY

In one aspect, provided herein is a composition or medical preparation comprising at least one RNA, wherein the at least one RNA encodes the following amino acid sequences: (i) an amino acid sequence comprising Kallikrein-2 (KLK2), an immunogenic variant thereof, or an immunogenic fragment of the KLK2 or the immunogenic variant thereof; (ii) an amino acid sequence comprising Prostate Specific Antigen (PSA), an immunogenic variant thereof, or an immunogenic fragment of the PSA or the immunogenic variant thereof; (iii) an amino acid sequence comprising Prostatic Acid Phosphatase (PAP), an immunogenic variant thereof, or an immunogenic fragment of the PAP or the immunogenic variant thereof; (iv) an amino acid sequence comprising Homeobox B13 (HOXB13), an immunogenic variant thereof, or an immunogenic fragment of the HOXB13 or the immunogenic variant thereof; and

(v) an amino acid sequence comprising NK3 Homeobox 1 (NKX3-1), an immunogenic variant thereof, or an immunogenic fragment of the NKX3-1 or the immunogenic variant thereof.

In one embodiment, each of the amino acid sequences under (i), (ii), (iii), (iv), or (v) is encoded by a separate RNA.

In one embodiment,

(i) the RNA encoding the amino acid sequence under (i) comprises the nucleotide sequence of SEQ ID NO: 3 or 4, or a nucleotide sequence having at least 99%, 98%, 97%, 96%, 95%, 90%, 85%, or 80% identity to the nucleotide sequence of SEQ ID NO: 3 or 4; and/or

(ii) the amino acid sequence under (i) comprises the amino acid sequence of SEQ ID NO: 1 or 2, or an amino acid sequence having at least 99%, 98%, 97%, 96%, 95%, 90%, 85%, or 80% identity to the amino acid sequence of SEQ ID NO: 1 or 2.

In one embodiment,

(i) the RNA encoding the amino acid sequence under (ii) comprises the nucleotide sequence of SEQ ID NO: 7 or 8, or a nucleotide sequence having at least 99%, 98%, 97%, 96%, 95%, 90%, 85%, or 80% identity to the nucleotide sequence of SEQ ID NO: 7 or 8; and/or

(ii) the amino acid sequence under (ii) comprises the amino acid sequence of SEQ ID NO: 5 or 6, or an amino acid sequence having at least 99%, 98%, 97%, 96%, 95%, 90%, 85%, or 80% identity to the amino acid sequence of SEQ ID NO: 5 or 6.

In one embodiment,

(i) the RNA encoding the amino acid sequence under (iii) comprises the nucleotide sequence of SEQ ID NO: 11 or 12, or a nucleotide sequence having at least 99%, 98%, 97%, 96%, 95%, 90%, 85%, or 80% identity to the nucleotide sequence of SEQ ID NO: 11 or 12; and/or

(ii) the amino acid sequence under (iii) comprises the amino acid sequence of SEQ ID NO: 9 or 10, or an amino acid sequence having at least 99%, 98%, 97%, 96%, 95%, 90%, 85%, or 80% identity to the amino acid sequence of SEQ ID NO: 9 or 10.

In one embodiment,

(i) the RNA encoding the amino acid sequence under (iv) comprises the nucleotide sequence of SEQ ID NO: 15 or 16, or a nucleotide sequence having at least 99%, 98%, 97%, 96%, 95%, 90%, 85%, or 80% identity to the nucleotide sequence of SEQ ID NO: 15 or 16; and/or

(ii) the amino acid sequence under (iv) comprises the amino acid sequence of SEQ ID NO: 13 or 14, or an amino acid sequence having at least 99%, 98%, 97%, 96%, 95%, 90%, 85%, or 80% identity to the amino acid sequence of SEQ ID NO: 13 or 14.

In one embodiment,

(i) the RNA encoding the amino acid sequence under (v) comprises the nucleotide sequence of SEQ ID NO: 19 or 20, or a nucleotide sequence having at least 99%, 98%, 97%, 96%, 95%, 90%, 85%, or 80% identity to the nucleotide sequence of SEQ ID NO: 19 or 20; and/or

(ii) the amino acid sequence under (v) comprises the amino acid sequence of SEQ ID NO: 17 or 18, or an amino acid sequence having at least 99%, 98%, 97%, 96%, 95%, 90%, 85%, or 80% identity to the amino acid sequence of SEQ ID NO: 17 or 18.

In one embodiment, at least one of the amino acid sequences under (i), (ii), (iii), (iv), or (v) is encoded by a coding sequence which is codon-optimized and/or the G/C content of which is increased compared to wild type coding sequence, wherein the codon-optimization and/or the increase in the G/C content preferably does not change the sequence of the encoded amino acid sequence. In one embodiment, each of the amino acid sequences under (i), (ii), (iii), (iv), or (v) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In one embodiment, at least one RNA is a modified RNA, in particular a stabilized mRNA. In one embodiment, at least one RNA comprises a modified nucleoside in place of at least one uridine. In one embodiment, at least one RNA comprises a modified nucleoside in place of each uridine. In one embodiment, each RNA comprises a modified nucleoside in place of at least one uridine. In one embodiment, each RNA comprises a modified nucleoside in place of each uridine. In one embodiment, the modified nucleoside is independently selected from pseudouridine (ψ), N1-methyl-pseudouridine (m1ψ), and 5-methyl-uridine (m5U).

In one embodiment, at least one RNA comprises the 5′-cap m₂^{7,2′-O}Gppₛp(5′)G. In one embodiment, each RNA comprises the 5′-cap m₂^{7,2′-O}Gppₛp(5′)G.

In one embodiment, at least one RNA comprises a 5′-UTR comprising the nucleotide sequence of SEQ ID NO: 21, or a nucleotide sequence having at least 99%, 98%, 97%, 96%, 95%, 90%, 85%, or 80% identity to the nucleotide sequence of SEQ ID NO: 21. In one embodiment, each RNA comprises a 5′-UTR comprising the nucleotide sequence of SEQ ID NO: 21, or a nucleotide sequence having at least 99%, 98%, 97%, 96%, 95%, 90%, 85%, or 80% identity to the nucleotide sequence of SEQ ID NO: 21.

In one embodiment, at least one amino acid sequence under (i), (ii), (iii), (iv), or (v) comprises an amino acid sequence enhancing antigen processing and/or presentation. In one embodiment, each amino acid sequence under (i), (ii), (iii), (iv), or (v) comprises an amino acid sequence enhancing antigen processing and/or presentation. In one embodiment, the amino acid sequence enhancing antigen processing and/or presentation comprises an amino acid sequence corresponding to the transmembrane and cytoplasmic domain of a MHC molecule, preferably a MHC class I molecule.

In one embodiment,

(i) the RNA encoding the amino acid sequence enhancing antigen processing and/or presentation comprises the nucleotide sequence of SEQ ID NO: 25, or a nucleotide sequence having at least 99%, 98%, 97%, 96%, 95%, 90%, 85%, or 80% identity to the nucleotide sequence of SEQ ID NO: 25; and/or

(ii) the amino acid sequence enhancing antigen processing and/or presentation comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24.

In one embodiment, the amino acid sequence enhancing antigen processing and/or presentation further comprises an amino acid sequence coding for a secretory signal peptide.

In one embodiment,

(i) the RNA encoding the secretory signal peptide comprises the nucleotide sequence of SEQ ID NO: 23, or a nucleotide sequence having at least 99%, 98%, 97%, 96%, 95%, 90%, 85%, or 80% identity to the nucleotide sequence of SEQ ID NO: 23; and/or

(ii) the secretory signal peptide comprises the amino acid sequence of SEQ ID NO: 22, or an amino acid sequence having at least 99%, 98%, 97%, 96%, 95%, 90%, 85%, or 80% identity to the amino acid sequence of SEQ ID NO: 22.

In one embodiment, at least one amino acid sequence under (i), (ii), (iii), (iv), or (v) comprises an amino acid sequence which breaks immunological tolerance. In one embodiment, each amino acid sequence under (i), (ii), (iii), (iv), or (v) comprises an amino acid sequence which breaks immunological tolerance. In one embodiment, the amino acid sequence which breaks immunological tolerance comprises helper epitopes, preferably tetanus toxoid-derived helper epitopes.

In one embodiment,

(i) the RNA encoding the amino acid sequence which breaks immunological tolerance comprises the nucleotide sequence of SEQ ID NO: 27, or a nucleotide sequence having at least 99%, 98%, 97%, 96%, 95%, 90%, 85%, or 80% identity to the nucleotide sequence of SEQ ID NO: 27; and/or

(ii) the amino acid sequence which breaks immunological tolerance comprises the amino acid sequence of SEQ ID NO: 26, or an amino acid sequence having at least 99%, 98%, 97%, 96%, 95%, 90%, 85%, or 80% identity to the amino acid sequence of SEQ ID NO: 26.

In one embodiment, at least one RNA comprises a 3′-UTR comprising the nucleotide sequence of SEQ ID NO: 28, or a nucleotide sequence having at least 99%, 98%, 97%, 96%, 95%, 90%, 85%, or 80% identity to the nucleotide sequence of SEQ ID NO: 28. In one embodiment, each RNA comprises a 3′-UTR comprising the nucleotide sequence of SEQ ID NO: 28, or a nucleotide sequence having at least 99%, 98%, 97%, 96%, 95%, 90%, 85%, or 80% identity to the nucleotide sequence of SEQ ID NO: 28.

In one embodiment, at least one RNA comprises a poly-A sequence. In one embodiment, each RNA comprises a poly-A sequence. In one embodiment, the poly-A sequence comprises at least 100 nucleotides. In one embodiment, the poly-A sequence comprises or consists of the nucleotide sequence of SEQ ID NO: 29.

In one embodiment, the RNA is formulated as a liquid, formulated as a solid, or a combination thereof. In one embodiment, the RNA is formulated for injection. In one embodiment, the RNA is formulated for intravenous administration. In one embodiment, the RNA is formulated or is to be formulated as lipoplex particles. In one embodiment, the RNA lipoplex particles are obtainable by mixing the RNA with liposomes.

In one embodiment, the composition or medical preparation is a pharmaceutical composition. In one embodiment, the pharmaceutical composition further comprises one or more pharmaceutically acceptable carriers, diluents and/or excipients.

In one embodiment, the medical preparation is a kit. In one embodiment, the RNAs and optionally the liposomes are in separate vials.

In one embodiment, the composition or medical preparation further comprises instructions for use of the RNAs and optionally the liposomes for treating or preventing prostate cancer. In one aspect, provided herein is the composition or medical preparation described herein for pharmaceutical use. In one embodiment, the pharmaceutical use comprises a therapeutic or prophylactic treatment of a disease or disorder. In one embodiment, the therapeutic or prophylactic treatment of a disease or disorder comprises treating or preventing prostate cancer. In one embodiment, the composition or medical preparation described herein is for administration to a human.

In one embodiment, the therapeutic or prophylactic treatment of a disease or disorder further comprises administering a further therapy. In one embodiment, the further therapy comprises one or more selected from the group consisting of: (i) surgery to excise, resect, or debulk a tumor, (ii) radiotherapy, and (iii) chemotherapy. In one embodiment, the further therapy comprises administering a further therapeutic agent. In one embodiment, the further therapeutic agent comprises an anti-cancer therapeutic agent. In one embodiment, the further therapeutic agent is a checkpoint modulator. In one embodiment, the checkpoint modulator is an anti-PD1 antibody, an anti-CTLA-4 antibody, or a combination of an anti-PD1 antibody and an anti-CTLA-4 antibody.

In one aspect, provided herein is a method of treating prostate cancer in a subject comprising administering at least one RNA to the subject, wherein the at least one RNA encodes the following amino acid sequences:

(i) an amino acid sequence comprising Kallikrein-2 (KLK2), an immunogenic variant thereof, or an immunogenic fragment of the KLK2 or the immunogenic variant thereof;

(ii) an amino acid sequence comprising Prostate Specific Antigen (PSA), an immunogenic variant thereof, or an immunogenic fragment of the PSA or the immunogenic variant thereof;

(iii) an amino acid sequence comprising Prostatic Acid Phosphatase (PAP), an immunogenic variant thereof, or an immunogenic fragment of the PAP or the immunogenic variant thereof;

(iv) an amino acid sequence comprising Homeobox B13 (HOXB13), an immunogenic variant thereof, or an immunogenic fragment of the HOXB13 or the immunogenic variant thereof; and

(v) an amino acid sequence comprising NK3 Homeobox 1 (NKX3-1), an immunogenic variant thereof, or an immunogenic fragment of the NKX3-1 or the immunogenic variant thereof.

In one embodiment, each of the amino acid sequences under (i), (ii), (iii), (iv), or (v) is encoded by a separate RNA.

In one embodiment,

(i) the RNA encoding the amino acid sequence under (i) comprises the nucleotide sequence of SEQ ID NO: 3 or 4, or a nucleotide sequence having at least 99%, 98%, 97%, 96%, 95%, 90%, 85%, or 80% identity to the nucleotide sequence of SEQ ID NO: 3 or 4; and/or

(ii) the amino acid sequence under (i) comprises the amino acid sequence of SEQ ID NO: 1 or 2, or an amino acid sequence having at least 99%, 98%, 97%, 96%, 95%, 90%, 85%, or 80% identity to the amino acid sequence of SEQ ID NO: 1 or 2.

In one embodiment,

(i) the RNA encoding the amino acid sequence under (ii) comprises the nucleotide sequence of SEQ ID NO: 7 or 8, or a nucleotide sequence having at least 99%, 98%, 97%, 96%, 95%, 90%, 85%, or 80% identity to the nucleotide sequence of SEQ ID NO: 7 or 8; and/or

(ii) the amino acid sequence under (ii) comprises the amino acid sequence of SEQ ID NO: 5 or 6, or an amino acid sequence having at least 99%, 98%, 97%, 96%, 95%, 90%, 85%, or 80% identity to the amino acid sequence of SEQ ID NO: 5 or 6.

In one embodiment,

(i) the RNA encoding the amino acid sequence under (iii) comprises the nucleotide sequence of SEQ ID NO: 11 or 12, or a nucleotide sequence having at least 99%, 98%, 97%, 96%, 95%, 90%, 85%, or 80% identity to the nucleotide sequence of SEQ ID NO: 11 or 12; and/or

(ii) the amino acid sequence under (iii) comprises the amino acid sequence of SEQ ID NO: 9 or 10, or an amino acid sequence having at least 99%, 98%, 97%, 96%, 95%, 90%, 85%, or 80% identity to the amino acid sequence of SEQ ID NO: 9 or 10.

In one embodiment,

(i) the RNA encoding the amino acid sequence under (iv) comprises the nucleotide sequence of SEQ ID NO: 15 or 16, or a nucleotide sequence having at least 99%, 98%, 97%, 96%, 95%, 90%, 85%, or 80% identity to the nucleotide sequence of SEQ ID NO: 15 or 16; and/or

(ii) the amino acid sequence under (iv) comprises the amino acid sequence of SEQ ID NO: 13 or 14, or an amino acid sequence having at least 99%, 98%, 97%, 96%, 95%, 90%, 85%, or 80% identity to the amino acid sequence of SEQ ID NO: 13 or 14.

In one embodiment,

(i) the RNA encoding the amino acid sequence under (v) comprises the nucleotide sequence of SEQ ID NO: 19 or 20, or a nucleotide sequence having at least 99%, 98%, 97%, 96%, 95%, 90%, 85%, or 80% identity to the nucleotide sequence of SEQ ID NO: 19 or 20; and/or

(ii) the amino acid sequence under (v) comprises the amino acid sequence of SEQ ID NO: 17 or 18, or an amino acid sequence having at least 99%, 98%, 97%, 96%, 95%, 90%, 85%, or 80% identity to the amino acid sequence of SEQ ID NO: 17 or 18.

In one embodiment, at least one of the amino acid sequences under (i), (ii), (iii), (iv), or (v) is encoded by a coding sequence which is codon-optimized and/or the G/C content of which is increased compared to wild type coding sequence, wherein the codon-optimization and/or the increase in the G/C content preferably does not change the sequence of the encoded amino acid sequence. In one embodiment, each of the amino acid sequences under (i), (ii), (iii), (iv), or (v) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In one embodiment, at least one RNA is a modified RNA, in particular a stabilized mRNA. In one embodiment, at least one RNA comprises a modified nucleoside in place of at least one uridine. In one embodiment, at least one RNA comprises a modified nucleoside in place of each uridine. In one embodiment, each RNA comprises a modified nucleoside in place of at least one uridine. In one embodiment, each RNA comprises a modified nucleoside in place of each uridine. In one embodiment, the modified nucleoside is independently selected from pseudouridine (ψ), N1-methyl-pseudouridine (m1ψ), and 5-methyl-uridine (m5U).

In one embodiment, at least one RNA comprises the 5′-cap m₂^{7,2′-O}Gpp₅p(5′)G. In one embodiment, each RNA comprises the 5′-cap m₂^{7,2′-O}Gpp₅p(5′)G.

In one embodiment, at least one RNA comprises a 5′-UTR comprising the nucleotide sequence of SEQ ID NO: 21, or a nucleotide sequence having at least 99%, 98%, 97%, 96%, 95%, 90%, 85%, or 80% identity to the nucleotide sequence of SEQ ID NO: 21. In one embodiment, each RNA comprises a 5′-UTR comprising the nucleotide sequence of SEQ ID NO: 21, or a nucleotide sequence having at least 99%, 98%, 97%, 96%, 95%, 90%, 85%, or 80% identity to the nucleotide sequence of SEQ ID NO: 21.

In one embodiment, at least one amino acid sequence under (i), (ii), (iii), (iv), or (v) comprises an amino acid sequence enhancing antigen processing and/or presentation. In one embodiment, each amino acid sequence under (i), (ii), (iii), (iv), or (v) comprises an amino acid sequence enhancing antigen processing and/or presentation. In one embodiment, the amino acid sequence enhancing antigen processing and/or presentation comprises an amino acid sequence corresponding to the transmembrane and cytoplasmic domain of a MHC molecule, preferably a MHC class I molecule.

In one embodiment,

(i) the RNA encoding the amino acid sequence enhancing antigen processing and/or presentation comprises the nucleotide sequence of SEQ ID NO: 25, or a nucleotide sequence having at least 99%, 98%, 97%, 96%, 95%, 90%, 85%, or 80% identity to the nucleotide sequence of SEQ ID NO: 25; and/or

(ii) the amino acid sequence enhancing antigen processing and/or presentation comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24.

In one embodiment, the amino acid sequence enhancing antigen processing and/or presentation further comprises an amino acid sequence coding for a secretory signal peptide.

In one embodiment,

(i) the RNA encoding the secretory signal peptide comprises the nucleotide sequence of SEQ ID NO: 23, or a nucleotide sequence having at least 99%, 98%, 97%, 96%, 95%, 90%, 85%, or 80% identity to the nucleotide sequence of SEQ ID NO: 23; and/or

(ii) the secretory signal peptide comprises the amino acid sequence of SEQ ID NO: 22, or an amino acid sequence having at least 99%, 98%, 97%, 96%, 95%, 90%, 85%, or 80% identity to the amino acid sequence of SEQ ID NO: 22.

In one embodiment, at least one amino acid sequence under (i), (ii), (iii), (iv), or (v) comprises an amino acid sequence which breaks immunological tolerance. In one embodiment, each amino acid sequence under (i), (ii), (iii), (iv), or (v) comprises an amino acid sequence which breaks immunological tolerance. In one embodiment, the amino acid sequence which breaks immunological tolerance comprises helper epitopes, preferably tetanus toxoid-derived helper epitopes.

In one embodiment,

(i) the RNA encoding the amino acid sequence which breaks immunological tolerance comprises the nucleotide sequence of SEQ ID NO: 27, or a nucleotide sequence having at least 99%, 98%, 97%, 96%, 95%, 90%, 85%, or 80% identity to the nucleotide sequence of SEQ ID NO: 27; and/or

(ii) the amino acid sequence which breaks immunological tolerance comprises the amino acid sequence of SEQ ID NO: 26, or an amino acid sequence having at least 99%, 98%, 97%, 96%, 95%, 90%, 85%, or 80% identity to the amino acid sequence of SEQ ID NO: 26.

In one embodiment, at least one RNA comprises a 3′-UTR comprising the nucleotide sequence of SEQ ID NO: 28, or a nucleotide sequence having at least 99%, 98%, 97%, 96%, 95%, 90%, 85%, or 80% identity to the nucleotide sequence of SEQ ID NO: 28. In one embodiment, each RNA comprises a 3′-UTR comprising the nucleotide sequence of SEQ ID NO: 28, or a nucleotide sequence having at least 99%, 98%, 97%, 96%, 95%, 90%, 85%, or 80% identity to the nucleotide sequence of SEQ ID NO: 28.

In one embodiment, at least one RNA comprises a poly-A sequence. In one embodiment, each RNA comprises a poly-A sequence. In one embodiment, the poly-A sequence comprises at least 100 nucleotides. In one embodiment, the poly-A sequence comprises or consists of the nucleotide sequence of SEQ ID NO: 29.

In one embodiment, the RNA is administered by injection. In one embodiment, the RNA is administered by intravenous administration.

In one embodiment, the RNA is formulated as lipoplex particles. In one embodiment, the RNA lipoplex particles are obtainable by mixing the RNA with liposomes.

In one embodiment, the subject is a human.

In one embodiment, the method described herein further comprises administering a further therapy. In one embodiment, the further therapy comprises one or more selected from the group consisting of: (i) surgery to excise, resect, or debulk a tumor, (ii) radiotherapy, and (iii) chemotherapy. In one embodiment, the further therapy comprises administering a further therapeutic agent. In one embodiment, the further therapeutic agent comprises an anti-cancer therapeutic agent. In one embodiment, the further therapeutic agent is a checkpoint modulator. In one embodiment, the checkpoint modulator is an anti-PD1 antibody, an anti-CTLA-4 antibody, or a combination of an anti-PD1 antibody and an anti-CTLA-4 antibody.

In one aspect, provided herein is RNA described herein, e.g.,

(i) RNA encoding an amino acid sequence comprising Kallikrein-2 (KLK2), an immunogenic variant thereof, or an immunogenic fragment of the KLK2 or the immunogenic variant thereof;

(ii) RNA encoding an amino acid sequence comprising Prostate Specific Antigen (PSA), an immunogenic variant thereof, or an immunogenic fragment of the PSA or the immunogenic variant thereof;

(iii) RNA encoding an amino acid sequence comprising Prostatic Acid Phosphatase (PAP), an immunogenic variant thereof, or an immunogenic fragment of the PAP or the immunogenic variant thereof;

(iv) RNA encoding an amino acid sequence comprising Homeobox B13 (HOXB13), an immunogenic variant thereof, or an immunogenic fragment of the HOXB13 or the immunogenic variant thereof; and/or

(v) RNA encoding an amino acid sequence comprising NK3 Homeobox 1 (NKX3-1), an immunogenic variant thereof, or an immunogenic fragment of the NKX3-1 or the immunogenic variant thereof, for use in a method described herein.

### BRIEF DESCRIPTION OF THE DRAWINGS

FIG. 1: General structure of the RNAs RBL038.1, RBL039.1, RBL040.1, RBL041.1, and RBL045.1.

Schematic illustration of the general structures of all RNA vaccines with 5′-cap, 5′- and 3′-untranslated regions (UTRs), coding sequences with N- and C-terminal fusion tags (sec and P2P16/MITD, respectively) and poly(A)-tail. Please note that the individual elements are not drawn exactly true to scale compared to their respective sequence lengths.

FIG. 2: 5′-capping structure beta-S-ARCA(D1) (m₂^{7,2′-O}GppSpG).

Shown in red are the differences between beta-S-ARCA(D1) and the basic cap analog m⁷GpppG: an —OCH₃ group at the C2′ position of the building block m⁷G and substitution of a non-bridging oxygen at the beta-phosphate by sulfur. Owing to the presence of a stereogenic P center (labeled with asterisk), the phosphorothioate cap analog beta-S-ARCA exists as two diastereomers. Based on their elution order in reversed phase HPLC, these have been designated as D1 and D2.

FIG. 3: Vector map of plasmid pST4-hAg-Kozak-KLK2-GS-P2P16-GS-MITD-FI-A30L70 for RBL038.1 production.

The insert with the sequence elements as labeled is shown in different colors. Eam1104I indicates the recognition site of the restriction endonuclease used for linearization. The Kanamycin resistance gene is shown in black.

FIG. 4: Vector map of plasmid pST4-hAg-Kozak-KLK3-GS-P2P16-GS-MITD-FI-A30L70 for RBL039.1 production.

The insert with the sequence elements as labeled is shown in different colors. Eam1104I indicates the recognition site of the restriction endonuclease used for linearization. The Kanamycin resistance gene is shown in black.

FIG. 5: Vector map of plasmid pST4-hAg-Kozak-ACPP-GS-P2P16-GS-MITD-FI-A30L70 for RBL40.1 production.

The insert with the sequence elements as labeled is shown in different colors. Eam1104I indicates the recognition site of the restriction endonuclease used for linearization. The Kanamycin resistance gene is shown in black.

FIG. 6: Vector map of plasmid pST4-hAg-Kozak-sec-GS-HOXB13-GS-P2P16-GS-MITD-FI-A30L70 for RBL041.1 production.

The insert with the sequence elements as labeled is shown in different colors. Eam1104I indicates the recognition site of the restriction endonuclease used for linearization. The Kanamycin resistance gene is shown in black.

FIG. 7: Vector map of plasmid pST4-hAg-Kozak-sec-GS-NKX3-1-GS-P2P16-GS-MITD-FI-A30L70 for RBL045.1 production.

The insert with the sequence elements as labeled is shown in different colors. Eam1104I indicates the recognition site of the restriction endonuclease used for linearization. The Kanamycin resistance gene is shown in black.

FIG. 8: Chemical structure of selected cationic lipids and co-lipids tested during formulation development.

FIG. 9: Organ selectivity of RNA lipoplexes with different charge ratios.

Positively charged luc-RNA lipoplexes show high luciferase expression in the lung, while negatively charged RNA lipoplexes show high selectivity of luciferase expression in the spleen.

FIG. 10: Biological activity of RNA lipoplexes depends on particle size and size of liposomes used for preparation.

20 μg luc-RNA was condensed with small (198 nm) and large (381 nm) liposomes for reconstitution of RNA lipoplexes (RNA(LIP)) and injected into BALB/c mice (n=5). Luciferase expression in the spleen was analyzed 6 h after luc-RNA(LIP) administration (mean±SD).

FIG. 11: Particle sizes of RNA lipoplexes reconstituted according to the clinical formulation protocol.

Particle sizes of RNA lipoplexes reconstituted by different experimenters, in different laboratories, and with different RNA constructs, were analyzed by PCS measurements. For experiments numbers 3 and 10, two independent preparations have been performed.

FIG. 12: Size and polydispersity index for RNA lipoplexes with different charge ratios.

Particle size (z-average) and polydispersity index were measured for RNA lipoplexes with different charge ratios (DOTMA:RNA) 10 min, 2 h, and 24 h after preparation.

FIG. 13: Size and biological activity of RNA lipoplexes with different charge ratios.

(A) Particle size (z-average) and polydispersity index was measured for RNA lipoplexes with different charge ratios (DOTMA:RNA) directly after preparation (10 min). (B) Luciferase expression in the spleen was analyzed 6 h after luc-RNA(LIP) (20 μg RNA) administration in BALB/c mice (n=4-5).

FIG. 14: Localization of bioluminescence signal after IV administration of luciferase RNA(LIP).

Bioluminescence imaging 6 h after intravenous injection of luc-RNA(LIP) (20 μg RNA) into BALB/c mice (n=3) in vivo (A) and of explanted spleen, liver as well as lungs ex vivo (B). One representative mouse is shown.

FIG. 15: RNA(LIP) is selectively internalized by splenic APCs.

BALB/c mice (n=3) were injected intravenously with Cy5-RNA (40 μg (HED: 9.48 mg)) formulated with rhodamine-labeled liposomes. Uptake of Cy5-labelled RNA (lower row) or Rhodamine-labeled liposomes (upper row) by cell populations in spleen was assessed by flow cytometry 1 h after lipoplex injection. Representative dot plots are shown.

FIG. 16: Break of tolerance and antigen-specific in vivo cytotoxicity following immunization with AH5-RNA(LIP).

BALB/c mice (n=5) were immunized intravenously with AH5-RNA(LIP) (40 μg RNA) on days 0, 3, 8, and 15 (green). The frequencies of antigen-specific CD8+ T cells were monitored in blood via gp70-MHC tetramer staining (grey). The line represents mean value of tetramer frequency (A). BALB/c mice (n=5) were immunized intravenously with AH5-RNA(LIP) (40 μg RNA) on days 0, 3, 8, or left untreated. On day 12, an in vivo cytotoxicity assay was performed by administration of a mixture of CFSEhigh (AH-5 peptide loaded) and CFSElow (Inf-HA peptide loaded) spleen cells from naïve BALB/c mice. AH5-specific lyses was shown for one representative mouse. All AH5-RNA(LIP) immunized mice showed over 90% antigen-specific lytic activity (B).

FIG. 17: Transient elevation of IFN-α after RNA(LIP) vaccination.

(A) C57BL/6 mice (n=3) were injected with HA-RNA(LIP) (40 μg RNA), liposomes alone or PBS as control. Serum concentrations of IFN-α and TNF-α were assessed via ELISA 6 h and 24 h after treatments (mean±SD). (B) Untouched or splenectomized C57BL/6 mice (n=2) were injected intravenously with HA-RNA(LIP) (40 μg RNA). Serum concentrations of IFN-α was assessed via ELISA 6 h after treatment (mean±SD).

FIG. 18: Vaccination with W_pro1 antigen RNAs leads to antigen-specific T-cell responses. Splenocytes of intravenously vaccinated A2/DR1 mice (n=4-5/group) were restimulated for 20 h with BMDCs electroporated with the corresponding mRNAs as indicated. BMDCs electroporated with irrelevant mRNA were used as control (open symbols, grey bars). Effector function was measured using an IFN-γ ELISPOT assay. Symbols indicate mean values of triplicate wells from individual animals. Bars indicate median of all animals per group.

FIG. 19: Mean levels of IFN-α (black bars) and IL-6 (grey bars) in animals of the high dose group.

Error bars show standard deviations. IL-6 induction was much stronger after the 1^{st} dosing (day 1) than after the 5^{th} dosing (day 22).

FIG. 20: Induction of antigen-specific T cells in the spleen by KLK2-, KLK3-, ACPP-, NKX3-1- and HOXB13-coding RNA.

IFN-γ ELISPOT analysis of T-cell effectors from the spleen of mice immunized with lipoplex-formulated RNA coding for KLK2(aa 1-261), KLK3(aa 1-261), ACPP(aa 1-418), HOXB13(aa 1-284) or NKX3-1(aa 1-234). Splenocytes obtained five days after the final immunization were re-stimulated with either a peptide pool spanning the respective human protein, P2/P16/P17 peptides or with an irrelevant control peptide CMV pp65(495-504). Dots represent individual animals; horizontal bars indicate the mean±SD of the three animals.

### DESCRIPTION OF THE SEQUENCES

The following table provides a listing of certain sequences referenced herein.

TABLE 1
DESCRIPTION OF THE SEQUENCES

| | | |
|---|---|---|
| SEQ | | |
| ID | | |
| NO: | Description | SEQUENCE |

KLK2

| | | |
|---|---|---|
| 1 | KLK2 (amino | MWDLVLSIALSVGCTGAVPLIQSRIVGGWECEKHSQPWQVAVYSHGWAHCGGVLVHPQWVLTAAHCLKKNSQVWLGRHNLFEPEDTGQRVP |
| | acid) | VSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYS |
| | | EKVTEFMLCAGLWTGGKDTCGGDSGGPLVCNGVLQGITSWGPEPCALPEKPAVYTKVVHYRKWIKDTIAANP |
| 2 | KLK2 fusion | MWDLVLSIALSVGCTGAVPLIQSRIVGGWECEKHSQPWQVAVYSHGWAHCGGVLVHPQWVLTAAHCLKKNSQVWLGRHNLFEPEDTGQRVP |
| | (amino acid) | VSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYS |
| | | EKVTEFMLCAGLWTGGKDTCGGDSGGPLVCNGVLQGITSWGPEPCALPEKPAVYTKVVHYRKWIKDTIAANPGGSGGGGSGGKKQYIKANSKFI |
| | | GITELKKLGGGKRGGGKKMTNSVDDALINSTKIYSYFPSVISKVNQGAQGKKLGSSGGGGSPGGGSSIVGIVAGLAVLAVVVIGAVVATVMCRRKS |
| | | SGGKGGSYSQAASSDSAQGSDVSLTA |
| 3 | KLK2 (CDS) | AUGUGGGACCUGGUUCUCUCCAUCGCCUUGUCUGUGGGGUGCACUGGUGCCGUGCCCCUCAUCCAGUCUCGGAUUGUGGGAGGCUG |
| | | GGAGUGUGAGAAGCAUUCCCAACCCUGGCAGGUGGCUGUGUACAGUCAUGGAUGGGCACACUGUGGGGGUGUCCUGGUGCACCCCCA |
| | | GUGGGUGCUCACAGCUGCCCAUUGCCUAAAGAAGAAUAGCCAGGUCUGGCUGGGUCGGCACAACCUGUUUGAGCCUGAAGACACAGG |
| | | CCAGAGGGUCCCUGUCAGCCACAGCUUCCCACACCCGCUCUACAAUAUGAGCCUUCUGAAGCAUCAAAGCCUUAGACCAGAUGAAGAC |
| | | UCCAGCCAUGACCUCAUGCUGCUCCGCCUGUCAGAGCCUGCCAAGAUCACAGAUGUUGUGAAGGUCCUGGGCCUGCCCACCCAGGAGC |
| | | CAGCACUGGGGACCACCUGCUACGCCUCAGGCUGGGGCAGCAUCGAACCAGAGGAGUUCUUGCGCCCCAGGAGUCUUCAGUGUGUGA |
| | | GCCUCCAUCUCCUGUCCAAUGACAUGUGUGCUAGAGCUUACUCUGAGAAGGUGACAGAGUUCAUGUUGUGUGCUGGGCUCUGGACA |
| | | GGUGGUAAAGACACUUGUGGGGGUGAUUCUGGGGGUCCACUUGUCUGUAAUGGUGUGCUUCAAGGUAUCACAUCAUGGGGCCCUG |
| | | AGCCAUGUGCCCUGCCUGAAAAGCCUGCUGUGUACACCAAGGUGGUGCAUUACCGGAAGUGGAUCAAGGACACCAUCGCAGCCAACCC |
| | | C |
| 4 | KLK2 (RNA) | GGGCGAACUAGUAUUCUUCUGGUCCCCACAGACUCAGAGAGAACCCGCCACCAUGUGGGACCUGGUUCUCUCCAUCGCCUUGUCUGUG |
| | | GGGUGCACUGGUGCCGUGCCCCUCAUCCAGUCUCGGAUUGUGGGAGGCUGGGAGUGUGAGAAGCAUUCCCAACCCUGGCAGGUGGCU |
| | | GUGUACAGUCAUGGAUGGGCACACUGUGGGGGUGUCCUGGUGCACCCCCAGUGGGUGCUCACAGCUGCCCAUUGCCUAAAGAAGAAU |
| | | AGCCAGGUCUGGCUGGGUCGGCACAACCUGUUUGAGCCUGAAGACACAGGCCAGAGGGUCCCUGUCAGCCACAGCUUCCCACACCCGC |
| | | UCUACAAUAUGAGCCUUCUGAAGCAUCAAAGCCUUAGACCAGAUGAAGACUCCAGCCAUGACCUCAUGCUGCUCCGCCUGUCAGAGCC |
| | | UGCCAAGAUCACAGAUGUUGUGAAGGUCCUGGGCCUGCCCACCCAGGAGCCAGCACUGGGGACCACCUGCUACGCCUCAGGCUGGGGC |
| | | AGCAUCGAACCAGAGGAGUUCUUGCGCCCCAGGAGUCUUCAGUGUGUGAGCCUCCAUCUCCUGUCCAAUGACAUGUGUGCUAGAGCU |
| | | UACUCUGAGAAGGUGACAGAGUUCAUGUUGUGUGCUGGGCUCUGGACAGGUGGUAAAGACACUUGUGGGGGUGAUUCUGGGGGUC |
| | | CACUUGUCUGUAAUGGUGUGCUUCAAGGUAUCACAUCAUGGGGCCCUGAGCCAUGUGCCCUGCCUGAAAAGCCUGCUGUGUACACCA |
| | | AGGUGGUGCAUUACCGGAAGUGGAUCAAGGACACCAUCGCAGCCAACCCCGGAGGAUCCGGUGGUGGCGGCAGCGGCGGCAAGAAGC |
| | | AGUACAUCAAGGCCAACAGCAAGUUCAUCGGCAUCACCGAGCUGAAGAAGCUGGGAGGGGGCAAACGGGGAGGCGGCAAAAAGAUGA |
| | | CCAACAGCGUGGACGACGCCCUGAUCAACAGCACCAAGAUCUACAGCUACUUCCCCAGCGUGAUCAGCAAAGUGAACCAGGGCGCUCAG |
| | | GGCAAGAAACUGGGCUCUAGCGGAGGGGGAGGCUCUCCUGGCGGGGGAUCUAGCAUCGUGGGAAUUGUGGCAGGACUGGCAGUGCU |
| | | GGCCGUGGUGGUGAUCGGAGCCGUGGUGGCUACCGUGAUGUGCAGACGGAAGUCCAGCGGAGGCAAGGGCGGCAGCUACAGCCAGGC |
| | | CGCCAGCUCUGAUAGCGCCCAGGGCAGCGACGUGUCACUGACAGCCUAGUAACUCGAGCUGGUACUGCAUGCACGCAAUGCUAGCUGC |
| | | CCCUUUCCCGUCCUGGGUACCCCGAGUCUCCCCCGACCUCGGGUCCCAGGUAUGCUCCCACCUCCACCUGCCCCACUCACCACCUCUGCU |
| | | AGUUCCAGACACCUCCCAAGCACGCAGCAAUGCAGCUCAAAACGCUUAGCCUAGCCACACCCCCACGGGAAACAGCAGUGAUUAACCUU |
| | | UAGCAAUAAACGAAAGUUUAACUAAGCUAUACUAACCCCAGGGUUGGUCAAUUUCGUGCCAGCCACACCGAGACCUGGUCCAGAGUC |
| | | GCUAGCCGCGUCGCUAAAAAAAAAAAAAAAAAAAAAAAAAAAAAAGCAUAUGACUAAAAAAAAAAAAAAAAAAAAAAAAAAAAAAAA |
| | | AAAAAAAAAAAAAAAAAAAAAAAAAAAAAAAAAAAAAA |

PSA

| | | |
|---|---|---|
| 5 | PSA (amino acid) | MWVPVVFLTLSVTWIGAAPLILSRIVGGWECEKHSQPWQVLVASRGRAVCGGVLVHPQWVLTAAHCIRNKSVILLGRHSLFHPEDTGQVFQVSH |
| | | SFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQ |
| | | KVTKFMLCAGRWTGGKSTCSGDSGGPLVCNGVLQGITSWGSEPCALPERPSLYTKVVHYRKWIKDTIVANP |
| 6 | PSA fusion | MWVPVVFLTLSVTWIGAAPLILSRIVGGWECEKHSQPWQVLVASRGRAVCGGVLVHPQWVLTAAHCIRNKSVILLGRHSLFHPEDTGQVFQVSH |
| | (amino acid) | SFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQ |
| | | KVTKFMLCAGRWTGGKSTCSGDSGGPLVCNGVLQGITSWGSEPCALPERPSLYTKVVHYRKWIKDTIVANPGGSGGGGSGGKKQYIKANSKFIGI |
| | | TELKKLGGGKRGGGKKMTNSVDDALINSTKIYSYFPSVISKVNQGAQGKKLGSSGGGGSPGGGSSIVGIVAGLAVLAVVVIGAVVATVMCRRKSS |
| | | GGKGGSYSQAASSDSAQGSDVSLTA |
| 7 | PSA (CDS) | AUGUGGGUCCCGGUUGUCUUCCUCACCCUGUCCGUGACGUGGAUUGGUGCUGCACCCCUCAUCCUGUCUCGGAUUGUGGGAGGCUGG |
| | | GAGUGCGAGAAGCAUUCCCAACCCUGGCAGGUGCUUGUGGCCUCUCGUGGCAGGGCAGUCUGCGGCGGUGUUCUGGUGCACCCCCAG |
| | | UGGGUCCUCACAGCUGCCCACUGCAUCAGGAACAAAAGCGUGAUCUUGCUGGGUCGGCACAGCCUGUUUCAUCCUGAAGACACAGGCC |
| | | AGGUAUUUCAGGUCAGCCACAGCUUCCCACACCCGCUCUACGAUAUGAGCCUCCUGAAGAAUCGAUUCCUCAGGCCAGGUGAUGACUC |
| | | CAGCCACGACCUCAUGCUGCUCCGCCUGUCAGAGCCUGCCGAGCUCACGGAUGCUGUGAAGGUCAUGGACCUGCCCACCCAGGAGCCAG |
| | | CACUGGGGACCACCUGCUACGCCUCAGGCUGGGGCAGCAUUGAACCAGAGGAGUUCUUGACCCCAAAGAAACUUCAGUGUGUGGACC |
| | | UCCAUGUUAUUUCCAAUGACGUGUGUGCGCAAGUUCACCCUCAGAAGGUGACCAAGUUCAUGCUGUGUGCUGGACGCUGGACAGGG |
| | | GGCAAAAGCACCUGCUCGGGUGAUUCUGGGGGCCCACUUGUCUGUAAUGGUGUGCUUCAAGGUAUCACGUCAUGGGGCAGUGAACC |
| | | AUGUGCCCUGCCCGAAAGGCCUUCCCUGUACACCAAGGUGGUGCAUUACCGGAAGUGGAUCAAGGACACCAUCGUGGCCAACCCC |
| 8 | PSA (RNA) | GGGCGAACUAGUAUUCUUCUGGUCCCCACAGACUCAGAGAGAACCCGCCACCAUGUGGGUCCCGGUUGUCUUCCUCACCCUGUCCGUG |
| | | ACGUGGAUUGGUGCUGCACCCCUCAUCCUGUCUCGGAUUGUGGGAGGCUGGGAGUGCGAGAAGCAUUCCCAACCCUGGCAGGUGCUU |
| | | GUGGCCUCUCGUGGCAGGGCAGUCUGCGGCGGUGUUCUGGUGCACCCCCAGUGGGUCCUCACAGCUGCCCACUGCAUCAGGAACAAAA |
| | | GCGUGAUCUUGCUGGGUCGGCACAGCCUGUUUCAUCCUGAAGACACAGGCCAGGUAUUUCAGGUCAGCCACAGCUUCCCACACCCGCU |
| | | CUACGAUAUGAGCCUCCUGAAGAAUCGAUUCCUCAGGCCAGGUGAUGACUCCAGCCACGACCUCAUGCUGCUCCGCCUGUCAGAGCCU |
| | | GCCGAGCUCACGGAUGCUGUGAAGGUCAUGGACCUGCCCACCCAGGAGCCAGCACUGGGGACCACCUGCUACGCCUCAGGCUGGGGCA |
| | | GCAUUGAACCAGAGGAGUUCUUGACCCCAAAGAAACUUCAGUGUGUGGACCUCCAUGUUAUUUCCAAUGACGUGUGUGCGCAAGUU |
| | | CACCCUCAGAAGGUGACCAAGUUCAUGCUGUGUGCUGGACGCUGGACAGGGGGCAAAAGCACCUGCUCGGGUGAUUCUGGGGGCCCA |
| | | CUUGUCUGUAAUGGUGUGCUUCAAGGUAUCACGUCAUGGGGCAGUGAACCAUGUGCCCUGCCCGAAAGGCCUUCCCUGUACACCAAG |
| | | GUGGUGCAUUACCGGAAGUGGAUCAAGGACACCAUCGUGGCCAACCCCGGAGGAUCCGGUGGUGGCGGCAGCGGCGGCAAGAAGCAG |
| | | UACAUCAAGGCCAACAGCAAGUUCAUCGGCAUCACCGAGCUGAAGAAGCUGGGAGGGGGCAAACGGGGAGGCGGCAAAAAGAUGACC |
| | | AACAGCGUGGACGACGCCCUGAUCAACAGCACCAAGAUCUACAGCUACUUCCCCAGCGUGAUCAGCAAAGUGAACCAGGGCGCUCAGG |
| | | GCAAGAAACUGGGCUCUAGCGGAGGGGGAGGCUCUCCUGGCGGGGGAUCUAGCAUCGUGGGAAUUGUGGCAGGACUGGCAGUGCUG |
| | | GCCGUGGUGGUGAUCGGAGCCGUGGUGGCUACCGUGAUGUGCAGACGGAAGUCCAGCGGAGGCAAGGGCGGCAGCUACAGCCAGGCC |
| | | GCCAGCUCUGAUAGCGCCCAGGGCAGCGACGUGUCACUGACAGCCUAGUAACUCGAGCUGGUACUGCAUGCACGCAAUGCUAGCUGCC |
| | | CCUUUCCCGUCCUGGGUACCCCGAGUCUCCCCCGACCUCGGGUCCCAGGUAUGCUCCCACCUCCACCUGCCCCACUCACCACCUCUGCUA |
| | | GUUCCAGACACCUCCCAAGCACGCAGCAAUGCAGCUCAAAACGCUUAGCCUAGCCACACCCCCACGGGAAACAGCAGUGAUUAACCUUU |
| | | AGCAAUAAACGAAAGUUUAACUAAGCUAUACUAACCCCAGGGUUGGUCAAUUUCGUGCCAGCCACACCGAGACCUGGUCCAGAGUCG |
| | | CUAGCCGCGUCGCUAAAAAAAAAAAAAAAAAAAAAAAAAAAAAAGCAUAUGACUAAAAAAAAAAAAAAAAAAAAAAAAAAAAAAAAA |
| | | AAAAAAAAAAAAAAAAAAAAAAAAAAAAAAAAAAAAA |

PAP

| | | |
|---|---|---|
| 9 | PAP (amino acid) | MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYK |
| | | HEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGL |
| | | HGQDLFGIWSKVYDPLYCESVHNFTLPSWATEDTMTKLRELSELSLLSLYGIHKQKEKSRLQGGVLVNEILNHMKRATQIPSYKKLIMYSAHDTTVS |
| | | GLQMALDVYNGLLPPYASCHLTELYFEKGEYFVEMYYRNETQHEPYPLMLPGCSPSCPLERFAELVGPVIPQDWSTECMTTNSHQVLKVIFAVAFC |
| | | LISAVLMVLLFIHIRRGLCWQRESYGNI |
| 10 | PAP fusion | MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYK |
| | (amino acid) | HEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGL |
| | | HGQDLFGIWSKVYDPLYCESVHNFTLPSWATEDTMTKLRELSELSLLSLYGIHKQKEKSRLQGGVLVNEILNHMKRATQIPSYKKLIMYSAHDTTVS |
| | | GLQMALDVYNGLLPPYASCHLTELYFEKGEYFVEMYYRNETQHEPYPLMLPGCSPSCPLERFAELVGPVIPQDWSTECMTTNSHQVLKVIFAVAFC |
| | | LISAVLMVLLFIHIRRGLCWQRESYGNIGGSGGGGSGGKKQYIKANSKFIGITELKKLGGGKRGGGKKMTNSVDDALINSTKIYSYFPSVISKVNQGA |
| | | QGKKLGSSGGGGSPGGGSSIVGIVAGLAVLAVVVIGAVVATVMCRRKSSGGKGGSYSQAASSDSAQGSDVSLTA |
| 11 | PAP (CDS) | AUGAGAGCUGCUCCCCUGCUGCUGGCAAGAGCUGCUUCUCUGUCUCUGGGAUUUCUGUUUCUGCUGUUUUUCUGGCUGGAUAGAUC |
| | | UGUGCUGGCAAAAGAACUGAAAUUUGUGACCCUGGUGUUUAGACACGGAGACAGAUCUCCCAUUGAUACCUUUCCCACCGAUCCCAU |
| | | CAAAGAAUCUUCUUGGCCACAGGGAUUUGGACAGCUGACCCAGCUGGGAAUGGAGCAGCACUACGAACUGGGAGAAUACAUCAGAAA |
| | | AAGAUACAGAAAAUUUCUGAAUGAAUCUUACAAACACGAACAGGUGUACAUCAGAUCUACCGAUGUGGAUAGAACCCUGAUGUCUG |
| | | CAAUGACCAAUCUGGCUGCUCUGUUUCCCCCAGAAGGAGUGAGCAUCUGGAAUCCCAUCCUGCUGUGGCAGCCCAUCCCUGUGCACAC |
| | | CGUGCCUCUGUCUGAAGAUCAGCUGCUGUACCUGCCUUUUAGAAAUUGCCCAAGAUUUCAGGAACUUGAAUCUGAAACCCUGAAAUC |
| | | UGAAGAAUUUCAGAAAAGACUGCACCCUUACAAAGAUUUUAUCGCAACCCUGGGAAAACUGUCUGGACUGCACGGACAGGAUCUCUU |
| | | UGGAAUUUGGUCAAAAGUGUACGAUCCUCUGUACUGUGAAUCUGUGCACAAUUUUACCCUGCCCUCUUGGGCAACCGAAGAUACCAU |
| | | GACCAAACUGAGAGAACUGUCUGAACUGUCUCUGCUGUCUCUGUACGGAAUCCACAAACAGAAAGAAAAAUCAAGACUGCAGGGAGG |
| | | AGUGCUGGUGAAUGAAAUCCUGAAUCACAUGAAAAGAGCAACCCAGAUCCCAUCUUACAAGAAGCUGAUCAUGUACUCUGCUCACGA |
| | | UACCACAGUGUCUGGACUGCAGAUGGCUCUGGAUGUGUACAAUGGACUGCUGCCUCCCUACGCUUCUUGCCACCUGACCGAACUGUA |
| | | CUUUGAAAAAGGAGAAUACUUUGUGGAAAUGUACUACAGAAAUGAAACCCAGCACGAACCUUACCCCCUGAUGCUGCCUGGAUGCUC |
| | | UCCCUCUUGCCCUCUGGAAAGAUUUGCUGAACUGGUGGGACCUGUGAUCCCUCAGGAUUGGUCCACAGAAUGCAUGACCACCAAUUC |
| | | UCACCAGGUGCUGAAAGUGAUCUUUGCUGUGGCUUUUUGCCUGAUCUCUGCUGUGCUGAUGGUGCUGCUGUUUAUCCACAUCAGGA |
| | | GAGGACUGUGCUGGCAGAGAGAAUCUUACGGAAAUAUC |
| 12 | PAP (RNA) | GGGCGAACUAGUAUUCUUCUGGUCCCCACAGACUCAGAGAGAACCCGCCACCAUGAGAGCUGCUCCCCUGCUGCUGGCAAGAGCUGCU |
| | | UCUCUGUCUCUGGGAUUUCUGUUUCUGCUGUUUUUCUGGCUGGAUAGAUCUGUGCUGGCAAAAGAACUGAAAUUUGUGACCCUGG |
| | | UGUUUAGACACGGAGACAGAUCUCCCAUUGAUACCUUUCCCACCGAUCCCAUCAAAGAAUCUUCUUGGCCACAGGGAUUUGGACAGC |
| | | UGACCCAGCUGGGAAUGGAGCAGCACUACGAACUGGGAGAAUACAUCAGAAAAAGAUACAGAAAAUUUCUGAAUGAAUCUUACAAAC |
| | | ACGAACAGGUGUACAUCAGAUCUACCGAUGUGGAUAGAACCCUGAUGUCUGCAAUGACCAAUCUGGCUGCUCUGUUUCCCCCAGAAG |
| | | GAGUGAGCAUCUGGAAUCCCAUCCUGCUGUGGCAGCCCAUCCCUGUGCACACCGUGCCUCUGUCUGAAGAUCAGCUGCUGUACCUGCC |
| | | UUUUAGAAAUUGCCCAAGAUUUCAGGAACUUGAAUCUGAAACCCUGAAAUCUGAAGAAUUUCAGAAAAGACUGCACCCUUACAAAGA |
| | | UUUUAUCGCAACCCUGGGAAAACUGUCUGGACUGCACGGACAGGAUCUCUUUGGAAUUUGGUCAAAAGUGUACGAUCCUCUGUACU |
| | | GUGAAUCUGUGCACAAUUUUACCCUGCCCUCUUGGGCAACCGAAGAUACCAUGACCAAACUGAGAGAACUGUCUGAACUGUCUCUGC |
| | | UGUCUCUGUACGGAAUCCACAAACAGAAAGAAAAAUCAAGACUGCAGGGAGGAGUGCUGGUGAAUGAAAUCCUGAAUCACAUGAAA |
| | | AGAGCAACCCAGAUCCCAUCUUACAAGAAGCUGAUCAUGUACUCUGCUCACGAUACCACAGUGUCUGGACUGCAGAUGGCUCUGGAU |
| | | GUGUACAAUGGACUGCUGCCUCCCUACGCUUCUUGCCACCUGACCGAACUGUACUUUGAAAAAGGAGAAUACUUUGUGGAAAUGUAC |
| | | UACAGAAAUGAAACCCAGCACGAACCUUACCCCCUGAUGCUGCCUGGAUGCUCUCCCUCUUGCCCUCUGGAAAGAUUUGCUGAACUGG |
| | | UGGGACCUGUGAUCCCUCAGGAUUGGUCCACAGAAUGCAUGACCACCAAUUCUCACCAGGUGCUGAAAGUGAUCUUUGCUGUGGCUU |
| | | UUUGCCUGAUCUCUGCUGUGCUGAUGGUGCUGCUGUUUAUCCACAUCAGGAGAGGACUGUGCUGGCAGAGAGAAUCUUACGGAAAU |
| | | AUCGGAGGAUCCGGUGGUGGCGGCAGCGGCGGCAAGAAGCAGUACAUCAAGGCCAACAGCAAGUUCAUCGGCAUCACCGAGCUGAAG |
| | | AAGCUGGGAGGGGGCAAACGGGGAGGCGGCAAAAAGAUGACCAACAGCGUGGACGACGCCCUGAUCAACAGCACCAAGAUCUACAGCU |
| | | ACUUCCCCAGCGUGAUCAGCAAAGUGAACCAGGGCGCUCAGGGCAAGAAACUGGGCUCUAGCGGAGGGGGAGGCUCUCCUGGCGGGG |
| | | GAUCUAGCAUCGUGGGAAUUGUGGCAGGACUGGCAGUGCUGGCCGUGGUGGUGAUCGGAGCCGUGGUGGCUACCGUGAUGUGCAG |
| | | ACGGAAGUCCAGCGGAGGCAAGGGCGGCAGCUACAGCCAGGCCGCCAGCUCUGAUAGCGCCCAGGGCAGCGACGUGUCACUGACAGCC |
| | | UAGUAACUCGAGCUGGUACUGCAUGCACGCAAUGCUAGCUGCCCCUUUCCCGUCCUGGGUACCCCGAGUCUCCCCCGACCUCGGGUCCC |
| | | AGGUAUGCUCCCACCUCCACCUGCCCCACUCACCACCUCUGCUAGUUCCAGACACCUCCCAAGCACGCAGCAAUGCAGCUCAAAACGCUU |
| | | AGCCUAGCCACACCCCCACGGGAAACAGCAGUGAUUAACCUUUAGCAAUAAACGAAAGUUUAACUAAGCUAUACUAACCCCAGGGUUG |
| | | GUCAAUUUCGUGCCAGCCACACCGAGACCUGGUCCAGAGUCGCUAGCCGCGUCGCUAAAAAAAAAAAAAAAAAAAAAAAAAAAAAAGC |
| | | AUAUGACUAAAAAAAAAAAAAAAAAAAAAAAAAAAAAAAAAAAAAAAAAAAAAAAAAAAAAAAAAAAAAAAAAAAAAA |

HOXB13

| | | |
|---|---|---|
| 13 | HOXB13 (amino | MEPGNYATLDGAKDIEGLLGAGGGRNLVAHSPLTSHPAAPTLMPAVNYAPLDLPGSAEPPKQCHPCPGVPQGTSPAPVPYGYFGGGYYSCRVSR |
| | acid) | SSLKPCAQAATLAAYPAETPTAGEEYPSRPTEFAFYPGYPGTYQPMASYLDVSVVQTLGAPGEPRHDSLLPVDSYQSWALAGGWNSQMCCQGE |
| | | QNPPGPFWKAAFADSSGQHPPDACAFRRGRKKRIPYSKGQLRELEREYAANKFITKDKRRKISAATSLSERQITIWFQNRRVKEKKVLAKVKNSAT |
| | | P |
| 14 | HOXB13 fusion | MRVMAPRTLILLLSGALALTETWAGSGGSGGGGSGGMEPGNYATLDGAKDIEGLLGAGGGRNLVAHSPLTSHPAAPTLMPAVNYAPLDLPGSAE |
| | (amino acid) | PPKQCHPCPGVPQGTSPAPVPYGYFGGGYYSCRVSRSSLKPCAQAATLAAYPAETPTAGEEYPSRPTEFAFYPGYPGTYQPMASYLDVSVVQTLG |
| | | APGEPRHDSLLPVDSYQSWALAGGWNSQMCCQGEQNPPGPFWKAAFADSSGQHPPDACAFRRGRKKRIPYSKGQLRELEREYAANKFITKDKR |
| | | RKISAATSLSERQITIWFQNRRVKEKKVLAKVKNSATPGGSGGGGSGGKKQYIKANSKFIGITELKKLGGGKRGGGKKMTNSVDDALINSTKIYSYF |
| | | PSVISKVNQGAQGKKLGSSGGGGSPGGGSSIVGIVAGLAVLAVVVIGAVVATVMCRRKSSGGKGGSYSQAASSDSAQGSDVSLTA |
| 15 | HOXB13 (CDS) | AUGGAGCCCGGCAAUUAUGCCACCUUGGAUGGAGCCAAGGAUAUCGAAGGCUUGCUGGGAGCGGGAGGGGGGCGGAAUCUGGUCGC |
| | | CCACUCCCCUCUGACCAGCCACCCAGCGGCGCCUACGCUGAUGCCUGCUGUCAACUAUGCCCCCUUGGAUCUGCCAGGCUCGGCGGAGC |
| | | CGCCAAAGCAAUGCCACCCAUGCCCUGGGGUGCCCCAGGGGACGUCCCCAGCUCCCGUGCCUUAUGGUUACUUUGGAGGCGGGUACUA |
| | | CUCCUGCCGAGUGUCCCGGAGUUCGCUGAAACCCUGUGCCCAGGCAGCCACCCUGGCCGCGUACCCCGCGGAGACUCCCACGGCCGGGG |
| | | AGGAGUACCCCAGCCGCCCCACUGAGUUUGCCUUCUAUCCGGGAUAUCCGGGAACCUACCAGCCUAUGGCCAGUUACCUGGACGUGUC |
| | | UGUGGUGCAGACUCUGGGUGCUCCUGGAGAACCGCGACAUGACUCCCUGUUGCCUGUGGACAGUUACCAGUCUUGGGCUCUCGCUGG |
| | | UGGCUGGAACAGCCAGAUGUGUUGCCAGGGAGAACAGAACCCACCAGGUCCCUUUUGGAAGGCAGCAUUUGCAGACUCCAGCGGGCA |
| | | GCACCCUCCUGACGCCUGCGCCUUUCGUCGCGGCCGCAAGAAACGCAUUCCGUACAGCAAGGGGCAGUUGCGGGAGCUGGAGCGGGAG |
| | | UAUGCGGCUAACAAGUUCAUCACCAAGGACAAGAGGCGCAAGAUCUCGGCAGCCACCAGCCUCUCGGAGCGCCAGAUUACCAUCUGGU |
| | | UUCAGAACCGCCGGGUCAAAGAGAAGAAGGUUCUCGCCAAGGUGAAGAACAGCGCUACCCCU |
| 16 | HOXB13(RNA) | GGGCGAACUAGUAUUCUUCUGGUCCCCACAGACUCAGAGAGAACCCGCCACCAUGAGAGUGAUGGCCCCCAGAACCCUGAUCCUGCUG |
| | | CUGUCUGGCGCCCUGGCCCUGACAGAGACAUGGGCCGGAAGCGGCGGCUCUGGAGGAGGCGGCUCCGGAGGCAUGGAGCCCGGCAAU |
| | | UAUGCCACCUUGGAUGGAGCCAAGGAUAUCGAAGGCUUGCUGGGAGCGGGAGGGGGGCGGAAUCUGGUCGCCCACUCCCCUCUGACC |
| | | AGCCACCCAGCGGCGCCUACGCUGAUGCCUGCUGUCAACUAUGCCCCCUUGGAUCUGCCAGGCUCGGCGGAGCCGCCAAAGCAAUGCCA |
| | | CCCAUGCCCUGGGGUGCCCCAGGGGACGUCCCCAGCUCCCGUGCCUUAUGGUUACUUUGGAGGCGGGUACUACUCCUGCCGAGUGUCC |
| | | CGGAGUUCGCUGAAACCCUGUGCCCAGGCAGCCACCCUGGCCGCGUACCCCGCGGAGACUCCCACGGCCGGGGAGGAGUACCCCAGCCG |
| | | CCCCACUGAGUUUGCCUUCUAUCCGGGAUAUCCGGGAACCUACCAGCCUAUGGCCAGUUACCUGGACGUGUCUGUGGUGCAGACUCU |
| | | GGGUGCUCCUGGAGAACCGCGACAUGACUCCCUGUUGCCUGUGGACAGUUACCAGUCUUGGGCUCUCGCUGGUGGCUGGAACAGCCA |
| | | GAUGUGUUGCCAGGGAGAACAGAACCCACCAGGUCCCUUUUGGAAGGCAGCAUUUGCAGACUCCAGCGGGCAGCACCCUCCUGACGCC |
| | | UGCGCCUUUCGUCGCGGCCGCAAGAAACGCAUUCCGUACAGCAAGGGGCAGUUGCGGGAGCUGGAGCGGGAGUAUGCGGCUAACAAG |
| | | UUCAUCACCAAGGACAAGAGGCGCAAGAUCUCGGCAGCCACCAGCCUCUCGGAGCGCCAGAUUACCAUCUGGUUUCAGAACCGCCGGG |
| | | UCAAAGAGAAGAAGGUUCUCGCCAAGGUGAAGAACAGCGCUACCCCUGGAGGAUCCGGUGGUGGCGGCAGCGGCGGCAAGAAGCAGU |
| | | ACAUCAAGGCCAACAGCAAGUUCAUCGGCAUCACCGAGCUGAAGAAGCUGGGAGGGGGCAAACGGGGAGGCGGCAAAAAGAUGACCA |
| | | ACAGCGUGGACGACGCCCUGAUCAACAGCACCAAGAUCUACAGCUACUUCCCCAGCGUGAUCAGCAAAGUGAACCAGGGCGCUCAGGG |
| | | CAAGAAACUGGGCUCUAGCGGAGGGGGAGGCUCUCCUGGCGGGGGAUCUAGCAUCGUGGGAAUUGUGGCAGGACUGGCAGUGCUGG |
| | | CCGUGGUGGUGAUCGGAGCCGUGGUGGCUACCGUGAUGUGCAGACGGAAGUCCAGCGGAGGCAAGGGCGGCAGCUACAGCCAGGCCG |
| | | CCAGCUCUGAUAGCGCCCAGGGCAGCGACGUGUCACUGACAGCCUAGUAACUCGAGCUGGUACUGCAUGCACGCAAUGCUAGCUGCCC |
| | | CUUUCCCGUCCUGGGUACCCCGAGUCUCCCCCGACCUCGGGUCCCAGGUAUGCUCCCACCUCCACCUGCCCCACUCACCACCUCUGCUAG |
| | | UUCCAGACACCUCCCAAGCACGCAGCAAUGCAGCUCAAAACGCUUAGCCUAGCCACACCCCCACGGGAAACAGCAGUGAUUAACCUUUA |
| | | GCAAUAAACGAAAGUUUAACUAAGCUAUACUAACCCCAGGGUUGGUCAAUUUCGUGCCAGCCACACCGAGACCUGGUCCAGAGUCGC |
| | | UAGCCGCGUCGCUAAAAAAAAAAAAAAAAAAAAAAAAAAAAAAGCAUAUGACUAAAAAAAAAAAAAAAAAAAAAAAAAAAAAAAAAA |
| | | AAAAAAAAAAAAAAAAAAAAAAAAAAAAAAAAAAAA |

NKX3-1

| | | |
|---|---|---|
| 17 | NKX3-1 (amino | MLRVPEPRPGEAKAEGAAPPTPSKPLTSFLIQDILRDGAQRQGGRTSSQRQRDPEPEPEPEPEGGRSRAGAQNDQLSTGPRAAPEEAETLAETEPE |
| | acid) | RHLGSYLLDSENTSGALPRLPQTPKQPQKRSRAAFSHTQVIELERKFSHQKYLSAPERAHLAKNLKLTETQVKIWFQNRRYKTKRKQLSSELGDLEKH |
| | | SSLPALKEEAFSRASLVSVYNSYPYYPYLYCVGSWSPAFW |
| 18 | NKX3-1 fusion | MRVMAPRTLILLLSGALALTETWAGSGGSGGGGSGGMLRVPEPRPGEAKAEGAAPPTPSKPLTSFLIQDILRDGAQRQGGRTSSQRQRDPEPEPE |
| | (amino acid) | PEPEGGRSRAGAQNDQLSTGPRAAPEEAETLAETEPERHLGSYLLDSENTSGALPRLPQTPKQPQKRSRAAFSHTQVIELERKFSHQKYLSAPERAH |
| | | LAKNLKLTETQVKIWFQNRRYKTKRKQLSSELGDLEKHSSLPALKEEAFSRASLVSVYNSYPYYPYLYCVGSWSPAFWGGSGGGGSGGKKQYIKAN |
| | | SKFIGITELKKLGGGKRGGGKKMTNSVDDALINSTKIYSYFPSVISKVNQGAQGKKLGSSGGGGSPGGGSSIVGIVAGLAVLAVVVIGAVVATVMC |
| | | RRKSSGGKGGSYSQAASSDSAQGSDVSLTA |
| 19 | NKX3-1 (CDS) | AUGCUGAGAGUGCCCGAACCUAGACCUGGCGAGGCUAAAGCUGAAGGCGCUGCUCCUCCUACACCUAGCAAGCCUCUGACCAGCUUCC |
| | | UGAUCCAAGACAUCCUGAGAGAUGGCGCCCAGAGACAAGGCGGCAGAACAAGCUCUCAGAGACAGAGAGAUCCCGAGCCUGAGCCUGA |
| | | ACCAGAACCUGAAGGCGGAAGAUCUAGAGCCGGCGCUCAGAACGAUCAGCUGAGCACAGGACCUAGAGCCGCUCCUGAGGAAGCCGAA |
| | | ACACUGGCCGAAACCGAGCCAGAGAGACACCUGGGCAGCUACCUGCUGGACAGCGAGAAUACUUCUGGCGCCCUGCCUAGACUGCCCCA |
| | | GACACCUAAACAGCCUCAGAAGCGGAGCAGAGCCGCCUUUAGCCACACACAAGUGAUCGAGCUGGAACGGAAGUUCAGCCACCAGAAG |
| | | UACCUGAGCGCCCCUGAAAGAGCCCACCUGGCCAAGAAUCUGAAGCUGACCGAGACUCAAGUGAAGAUCUGGUUCCAGAACCGGCGGU |
| | | ACAAGACCAAGCGGAAGCAGCUGUCAAGCGAGCUGGGCGACCUGGAAAAGCACAGCUCUCUGCCCGCUCUGAAAGAGGAAGCCUUCUC |
| | | CAGAGCCAGCCUGGUGUCCGUGUACAACAGCUACCCUUACUACCCCUACCUGUACUGCGUCGGCUCUUGGAGCCCUGCCUUUUGG |
| 20 | NKX3-1 (RNA) | GGGCGAACUAGUAUUCUUCUGGUCCCCACAGACUCAGAGAGAACCCGCCACCAUGAGAGUGAUGGCCCCCAGAACCCUGAUCCUGCUG |
| | | CUGUCUGGCGCCCUGGCCCUGACAGAGACAUGGGCCGGAAGCGGCGGCUCUGGAGGAGGCGGCUCCGGAGGCAUGCUGAGAGUGCCC |
| | | GAACCUAGACCUGGCGAGGCUAAAGCUGAAGGCGCUGCUCCUCCUACACCUAGCAAGCCUCUGACCAGCUUCCUGAUCCAAGACAUCC |
| | | UGAGAGAUGGCGCCCAGAGACAAGGCGGCAGAACAAGCUCUCAGAGACAGAGAGAUCCCGAGCCUGAGCCUGAACCAGAACCUGAAGG |
| | | CGGAAGAUCUAGAGCCGGCGCUCAGAACGAUCAGCUGAGCACAGGACCUAGAGCCGCUCCUGAGGAAGCCGAAACACUGGCCGAAACC |
| | | GAGCCAGAGAGACACCUGGGCAGCUACCUGCUGGACAGCGAGAAUACUUCUGGCGCCCUGCCUAGACUGCCCCAGACACCUAAACAGCC |
| | | UCAGAAGCGGAGCAGAGCCGCCUUUAGCCACACACAAGUGAUCGAGCUGGAACGGAAGUUCAGCCACCAGAAGUACCUGAGCGCCCCU |
| | | GAAAGAGCCCACCUGGCCAAGAAUCUGAAGCUGACCGAGACUCAAGUGAAGAUCUGGUUCCAGAACCGGCGGUACAAGACCAAGCGGA |
| | | AGCAGCUGUCAAGCGAGCUGGGCGACCUGGAAAAGCACAGCUCUCUGCCCGCUCUGAAAGAGGAAGCCUUCUCCAGAGCCAGCCUGGU |
| | | GUCCGUGUACAACAGCUACCCUUACUACCCCUACCUGUACUGCGUCGGCUCUUGGAGCCCUGCCUUUUGGGGAGGAUCCGGUGGUGG |
| | | CGGCAGCGGCGGCAAGAAGCAGUACAUCAAGGCCAACAGCAAGUUCAUCGGCAUCACCGAGCUGAAGAAGCUGGGAGGGGGCAAACG |
| | | GGGAGGCGGCAAAAAGAUGACCAACAGCGUGGACGACGCCCUGAUCAACAGCACCAAGAUCUACAGCUACUUCCCCAGCGUGAUCAGC |
| | | AAAGUGAACCAGGGCGCUCAGGGCAAGAAACUGGGCUCUAGCGGAGGGGGAGGCUCUCCUGGCGGGGGAUCUAGCAUCGUGGGAAU |
| | | UGUGGCAGGACUGGCAGUGCUGGCCGUGGUGGUGAUCGGAGCCGUGGUGGCUACCGUGAUGUGCAGACGGAAGUCCAGCGGAGGCA |
| | | AGGGCGGCAGCUACAGCCAGGCCGCCAGCUCUGAUAGCGCCCAGGGCAGCGACGUGUCACUGACAGCCUAGUAACUCGAGCUGGUACU |
| | | GCAUGCACGCAAUGCUAGCUGCCCCUUUCCCGUCCUGGGUACCCCGAGUCUCCCCCGACCUCGGGUCCCAGGUAUGCUCCCACCUCCACC |
| | | UGCCCCACUCACCACCUCUGCUAGUUCCAGACACCUCCCAAGCACGCAGCAAUGCAGCUCAAAACGCUUAGCCUAGCCACACCCCCACGG |
| | | GAAACAGCAGUGAUUAACCUUUAGCAAUAAACGAAAGUUUAACUAAGCUAUACUAACCCCAGGGUUGGUCAAUUUCGUGCCAGCCAC |
| | | ACCGAGACCUGGUCCAGAGUCGCUAGCCGCGUCGCUAAAAAAAAAAAAAAAAAAAAAAAAAAAAAAGCAUAUGACUAAAAAAAAAAA |
| | | AAAAAAAAAAAAAAAAAAAAAAAAAAAAAAAAAAAAAAAAAAAAAAAAAAAAAAAAAAA |

5′-UTR (hAg-Kozak)

| | | |
|---|---|---|
| 21 | 5′-UTR | GGGCGAACUAGUAUUCUUCUGGUCCCCACAGACUCAGAGAGAACCCGCCACC |

Sec/MITD

| | | |
|---|---|---|
| 22 | Sec (amino acid) | MRVMAPRTLILLLSGALALTETWAGS |
| 23 | Sec (CDS) | AUGAGAGUGAUGGCCCCCAGAACCCUGAUCCUGCUGCUGUCUGGCGCCCUGGCCCUGACAGAGACAUGGGCCGGAAGC |
| 24 | MITD (amino | IVGIVAGLAVLAVVVIGAVVATVMCRRKSSGGKGGSYSQAASSDSAQGSDVSLTA |
| | acid) | |
| 25 | MITD (CDS) | AUCGUGGGAAUUGUGGCAGGACUGGCAGUGCUGGCCGUGGUGGUGAUCGGAGCCGUGGUGGCUACCGUGAUGUGCAGACGGAAGU |
| | | CCAGCGGAGGCAAGGGCGGCAGCUACAGCCAGGCCGCCAGCUCUGAUAGCGCCCAGGGCAGCGACGUGUCACUGACAGCCUAGUAA |

P2P16

| | | |
|---|---|---|
| 26 | P2P16 (amino | KKQYIKANSKFIGITELKKLGGGKRGGGKKMTNSVDDALINSTKIYSYFPSVISKVNQGAQGKKL |
| | acid) | |
| 27 | P2P16 (CDS) | AAGAAGCAGUACAUCAAGGCCAACAGCAAGUUCAUCGGCAUCACCGAGCUGAAGAAGCUGGGAGGGGGCAAACGGGGAGGCGGCAAA |
| | | AAGAUGACCAACAGCGUGGACGACGCCCUGAUCAACAGCACCAAGAUCUACAGCUACUUCCCCAGCGUGAUCAGCAAAGUGAACCAGG |
| | | GCGCUCAGGGCAAGAAACUG |

3′-UTR (F1 element)

| | | |
|---|---|---|
| 28 | 3′-UTR | CUCGAGCUGGUACUGCAUGCACGCAAUGCUAGCUGCCCCUUUCCCGUCCUGGGUACCCCGAGUCUCCCCCGACCUCGGGUCCCAGGUAU |
| | | GCUCCCACCUCCACCUGCCCCACUCACCACCUCUGCUAGUUCCAGACACCUCCCAAGCACGCAGCAAUGCAGCUCAAAACGCUUAGCCUA |
| | | GCCACACCCCCACGGGAAACAGCAGUGAUUAACCUUUAGCAAUAAACGAAAGUUUAACUAAGCUAUACUAACCCCAGGGUUGGUCAAU |
| | | UUCGUGCCAGCCACACCGAGACCUGGUCCAGAGUCGCUAGCCGCGUCGCU |

A30L70

| | | |
|---|---|---|
| 29 | A30L70 | AAAAAAAAAAAAAAAAAAAAAAAAAAAAAAGCAUAUGACUAAAAAAAAAAAAAAAAAAAAAAAAAAAAAAAAAAAAAAAAAAAAAAA |
| | | AAAAAAAAAAAAAAAAAAAAAAA |

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

### Definitions

In the following, definitions will be provided which apply to all aspects of the present disclosure. The following terms have the following meanings unless otherwise indicated. Any undefined terms have their art recognized meanings.

Terms such as “reduce” or “inhibit” as used herein means the ability to cause an overall decrease, for example, of about 5% or greater, about 10% or greater, about 20% or greater, about 50% or greater, or about 75% or greater, in the level. The term “inhibit” or similar phrases includes a complete or essentially complete inhibition, i.e., a reduction to zero or essentially to zero.

Terms such as “increase” or “enhance” in one embodiment relate to an increase or enhancement by at least about 10%, at least about 20%, at least about 30%, at least about 40%, at least about 50%, at least about 80%, or at least about 100%. “Physiological pH” as used herein refers to a pH of about 7.5.

The term “ionic strength” refers to the mathematical relationship between the number of different kinds of ionic species in a particular solution and their respective charges. Thus, ionic strength I is represented mathematically by the formula

$$I = \frac{1}{2} \cdot {\sum }_{i} {z}_{i}^{2} \cdot {c}_{i}$$

in which c is the molar concentration of a particular ionic species and z the absolute value of its charge. The sum Σ is taken over all the different kinds of ions (i) in solution.

According to the disclosure, the term “ionic strength” in one embodiment relates to the presence of monovalent ions. Regarding the presence of divalent ions, in particular divalent cations, their concentration or effective concentration (presence of free ions) due to the presence of chelating agents is in one embodiment sufficiently low so as to prevent degradation of the RNA. In one embodiment, the concentration or effective concentration of divalent ions is below the catalytic level for hydrolysis of the phosphodiester bonds between RNA nucleotides. In one embodiment, the concentration of free divalent ions is 20 μM or less. In one embodiment, there are no or essentially no free divalent ions.

The term “freezing” relates to the solidification of a liquid, usually with the removal of heat.

The term “lyophilizing” or “lyophilization” refers to the freeze-drying of a substance by freezing it and then reducing the surrounding pressure to allow the frozen medium in the substance to sublimate directly from the solid phase to the gas phase.

The term “spray-drying” refers to spray-drying a substance by mixing (heated) gas with a fluid that is atomized (sprayed) within a vessel (spray dryer), where the solvent from the formed droplets evaporates, leading to a dry powder.

The term “cryoprotectant” relates to a substance that is added to a formulation in order to protect the active ingredients during the freezing stages.

The term “lyoprotectant” relates to a substance that is added to a formulation in order to protect the active ingredients during the drying stages.

The term “reconstitute” relates to adding a solvent such as water to a dried product to return it to a liquid state such as its original liquid state.

The term “recombinant” in the context of the present disclosure means “made through genetic engineering”. In one embodiment, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

In the context of the present disclosure, the term “particle” relates to a structured entity formed by molecules or molecule complexes. In one embodiment, the term “particle” relates to a micro- or nano-sized structure, such as a micro- or nano-sized compact structure. In the context of the present disclosure, the term “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

As used in the present disclosure, “nanoparticle” refers to a particle comprising RNA and at least one cationic lipid and having an average diameter suitable for intravenous administration.

The term “average diameter” refers to the mean hydrodynamic diameter of particles as measured by dynamic light scattering (DLS) with data analysis using the so-called cumulant algorithm, which provides as results the so-called Z_{average} with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

The term “polydispersity index” is used herein as a measure of the size distribution of an ensemble of particles, e.g., nanoparticles. The polydispersity index is calculated based on dynamic light scattering measurements by the so-called cumulant analysis.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in one embodiment, formed as follows: an ethanol solution comprising lipids, such as cationic lipids like DOTMA and additional lipids, is injected into an aqueous solution under stirring. In one embodiment, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

The prostate is a small gland found in a man's lower abdomen. It's located under the bladder and surrounding the urethra. The prostate is regulated by the hormone testosterone and produces seminal fluid, also known as semen. Semen is the substance containing sperm that exits the urethra during ejaculation.

As used herein, “prostate cancer,” is cancer in the prostate. When an abnormal, malignant growth of cells—which is called a tumor—forms in the prostate, it's called prostate cancer. Most prostate cancers are slow growing; however, some grow relatively quickly. The cancer cells may spread from the prostate to other areas of the body, particularly the bones and lymph nodes. It may initially cause no symptoms. In later stages, it can lead to difficulty urinating, blood in the urine or pain in the pelvis, back, or when urinating. About 99% of cases occur in males over the age of 50. Many cases are managed with active surveillance or watchful waiting. Other treatments may include a combination of surgery, radiation therapy, hormone therapy or chemotherapy. When it only occurs inside the prostate, it may be curable. In those in whom the disease has spread to the bones, pain medications, bisphosphonates and targeted therapy, among others, may be useful. Outcomes depend on a person's age and other health problems as well as how aggressive and extensive the cancer is. Globally, it is the second most common type of cancer and the fifth leading cause of cancer-related death in men.

The term “co-administered” or “co-administration” or the like as used herein refers to administration of two or more agents concurrently, simultaneously, or essentially at the same time, either as part of a single formulation or as multiple formulations that are administered by the same or different routes. “Essentially at the same time” as used herein means within about 1 minute, 5 minutes, 10 minutes, 15 minutes, 30 minutes, 1 hour, 2 hours, or 6 hours period of each other.

The disclosure describes nucleic acid sequences and amino acid sequences having a certain degree of identity to a given nucleic acid sequence or amino acid sequence, respectively (a reference sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. One important property includes an immunogenic property, in particular when administered to a subject. In some embodiments, a nucleic acid sequence or amino acid sequence having a particular degree of identity to a given nucleic acid sequence or amino acid sequence is functionally equivalent to the given sequence.

RNA

In the present disclosure, the term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA.

In certain embodiments of the present disclosure, the RNA is messenger RNA (mRNA) that relates to a RNA transcript which encodes a peptide or protein. As established in the art, mRNA generally contains a 5′-untranslated region (5′-UTR), a peptide coding region and a 3′-untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In one embodiment, the RNA may have modified nucleosides. In some embodiments, the RNA comprises a modified nucleoside in place of at least one (e.g., every) uridine. The term “uracil,” as used herein, describes one of the nucleobases that can occur in the nucleic acid of RNA. The structure of uracil is:

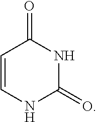

The term “uridine,” as used herein, describes one of the nucleosides that can occur in RNA. The structure of uridine is:

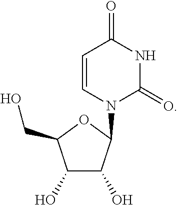 [SMILES: O=c1ccn([C@@H]2O[C@H](CO)[C@@H](O)[C@H]2O)c(=O)[nH]1]

UTP (uridine 5′-triphosphate) has the following structure:

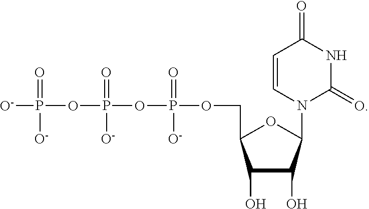

Pseudo-UTP (pseudouridine 5′-triphosphate) has the following structure:

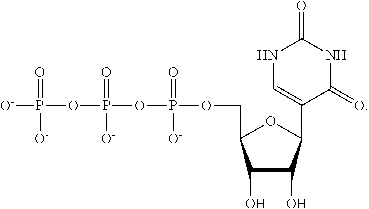

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m14W), which has the structure:

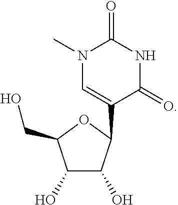 [SMILES: Cn1cc([C@@H]2O[C@H](CO)[C@@H](O)[C@H]2O)c(=O)[nH]c1=O]

N1-methyl-pseudo-UTP has the following structure:

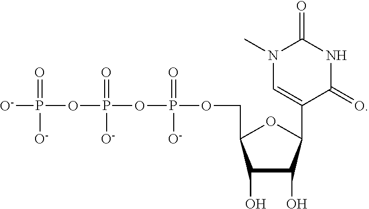

Another exemplary modified nucleoside is 5-methyl-uridine (m5U), which has the structure:

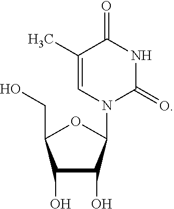

In some embodiments, one or more uridine in the RNA described herein is replaced by a modified nucleoside. In some embodiments, the modified nucleoside is a modified uridine. In some embodiments, the modified uridine replacing uridine is pseudouridine (ψ), N1-methyl-pseudouridine (m1ψ), or 5-methyl-uridine (m5U).

In some embodiments, the modified nucleoside replacing one or more uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³ψ), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Urn), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, at least one RNA comprises a modified nucleoside in place of at least one uridine. In some embodiments, at least one RNA comprises a modified nucleoside in place of each uridine. In some embodiments, each RNA comprises a modified nucleoside in place of at least one uridine. In some embodiments, each RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, at least one RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the RNA according to the present disclosure comprises a 5′-cap. In one embodiment, the RNA of the present disclosure does not have uncapped 5′-triphosphates. In one embodiment, the RNA may be modified by a 5′-cap analog. The term “5′-cap” refers to a structure found on the 5′-end of an mRNA molecule and generally consists of a guanosine nucleotide connected to the mRNA via a 5′- to 5′-triphosphate linkage. In one embodiment, this guanosine is methylated at the 7-position. Providing an RNA with a 5′-cap or 5′-cap analog may be achieved by in vitro transcription, in which the 5′-cap is co-transcriptionally expressed into the RNA strand, or may be attached to RNA post-transcriptionally using capping enzymes. In some embodiments, the building block cap for RNA is m₂^{7,3′-O}Gppp(m₁^{2′-O})ApG (also sometimes referred to as m₂^{7,3′-O}G(5′)ppp(5′)m^{2′-O}ApG), which has the following structure:

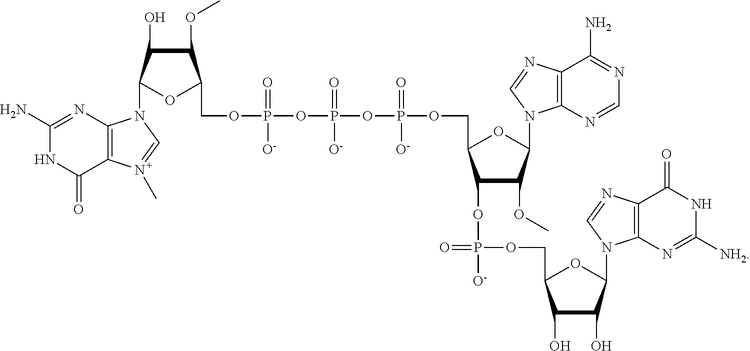

Below is an exemplary Cap1 RNA, which comprises RNA and m₂^{7,3′O}G(5′)ppp(5′)m^{2′-O}ApG:

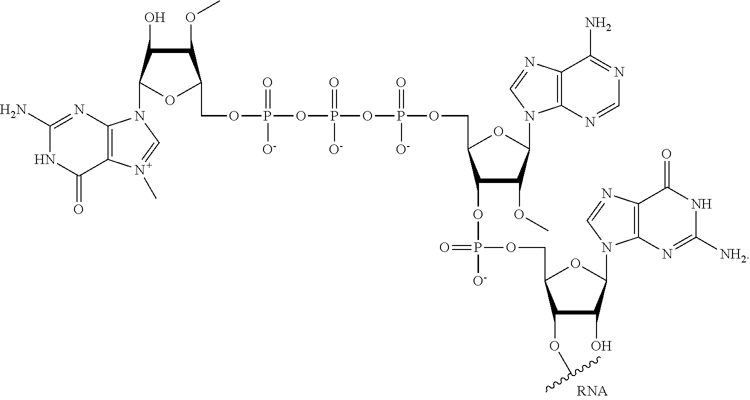

Below is another exemplary Cap1 RNA (no cap analog):

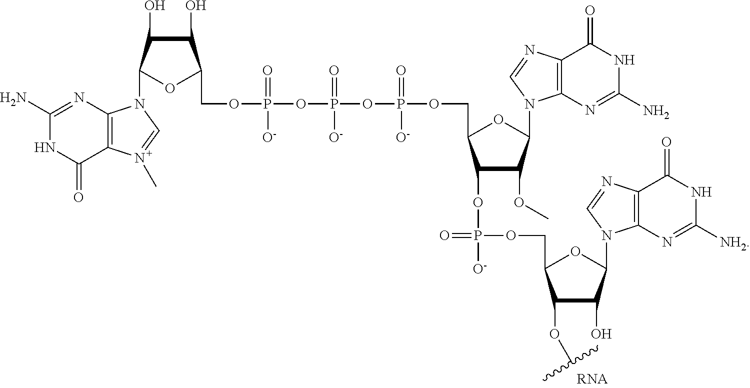

In some embodiments, the RNA is modified with “Cap0” structures using, in one embodiment, the cap analog anti-reverse cap (ARCA Cap (m₂^{7,3′O}G(5′)ppp(5′)G)) with the structure:

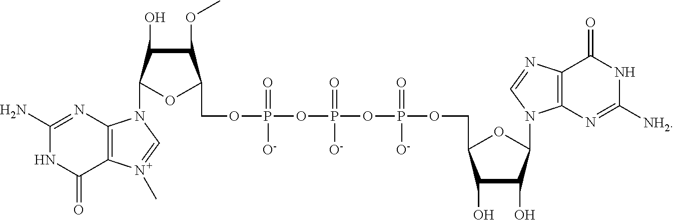

Below is an exemplary Cap0 RNA comprising RNA and m₂^{7,3′O}G(5′)ppp(5′)G:

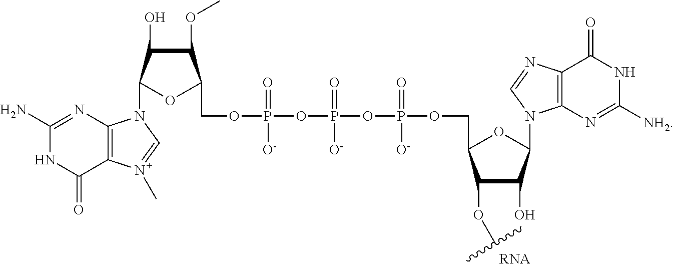

In some embodiments, the “Cap0” structures are generated using the cap analog Beta-S-ARCA (m₂^{7,2′O}G(5′)ppSp(5′)G) with the structure:

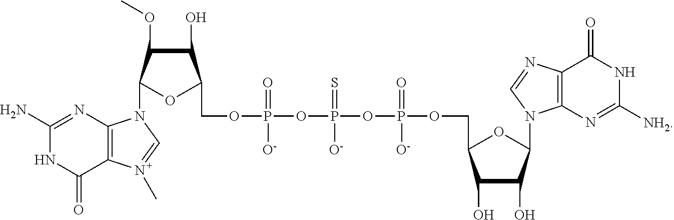

Below is an exemplary Cap0 RNA comprising Beta-S-ARCA (m₂^{7,2′O}G(5′)ppSp(5′)G) and RNA:

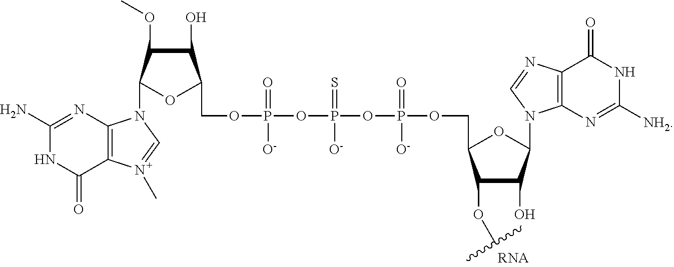

A particularly preferred Cap comprises the 5′-cap m₂^{7,2′O}G(5′)ppSp(5′)G. In some embodiments, at least one RNA described herein comprises the 5′-cap m₂^{7,2′O}G(5′)ppSp(5′)G. In some embodiments, each RNA described herein comprises the 5′-cap m₂^{7,2′O}G(5′)ppSp(5′)G. In some embodiments, RNA according to the present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′-end, upstream of the start codon of a protein-encoding region. A 5′-UTR is downstream of the 5′-cap (if present), e.g., directly adjacent to the 5′-cap. A 3′-UTR, if present, is located at the 3′-end, downstream of the termination codon of a protein-encoding region, but the term “3′-UTR” does preferably not include the poly-A sequence. Thus, the 3′-UTR is upstream of the poly-A sequence (if present), e.g., directly adjacent to the poly-A sequence.

A particularly preferred 5′-UTR comprises the nucleotide sequence of SEQ ID NO: 21. A particularly preferred 3′-UTR comprises the nucleotide sequence of SEQ ID NO: 28. In some embodiments, at least one RNA comprises a 5′-UTR comprising the nucleotide sequence of SEQ ID NO: 21, or a nucleotide sequence having at least 99%, 98%, 97%, 96%, 95%, 90%, 85%, or 80% identity to the nucleotide sequence of SEQ ID NO: 21. In some embodiments, each RNA comprises a 5′-UTR comprising the nucleotide sequence of SEQ ID NO: 21, or a nucleotide sequence having at least 99%, 98%, 97%, 96%, 95%, 90%, 85%, or 80% identity to the nucleotide sequence of SEQ ID NO: 21.

In some embodiments, at least one RNA comprises a 3′-UTR comprising the nucleotide sequence of SEQ ID NO: 28, or a nucleotide sequence having at least 99%, 98%, 97%, 96%, 95%, 90%, 85%, or 80% identity to the nucleotide sequence of SEQ ID NO: 28. In some embodiments, each RNA comprises a 3′-UTR comprising the nucleotide sequence of SEQ ID NO: 28, or a nucleotide sequence having at least 99%, 98%, 97%, 96%, 95%, 90%, 85%, or 80% identity to the nucleotide sequence of SEQ ID NO: 28.

As used herein, the term “poly-A tail” or “poly-A sequence” refers to an uninterrupted or interrupted sequence of adenylate residues which is typically located at the 3′-end of an RNA molecule. Poly-A tails or poly-A sequences are known to those of skill in the art and may follow the 3′-UTR in the RNAs described herein. An uninterrupted poly-A tail is characterized by consecutive adenylate residues. In nature, an uninterrupted poly-A tail is typical. RNAs disclosed herein can have a poly-A tail attached to the free 3′-end of the RNA by a template-independent RNA polymerase after transcription or a poly-A tail encoded by DNA and transcribed by a template-dependent RNA polymerase.

It has been demonstrated that a poly-A tail of about 120 A nucleotides has a beneficial influence on the levels of RNA in transfected eukaryotic cells, as well as on the levels of protein that is translated from an open reading frame that is present upstream (5′) of the poly-A tail (Holtkamp et al., 2006, Blood, vol. 108, pp. 4009-4017).

The poly-A tail may be of any length. In some embodiments, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette. In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an RNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly-A tail at its 3′-end, i.e., the poly-A tail is not masked or followed at its 3′-end by a nucleotide other than A. In some embodiments, a poly-A tail comprises the sequence of SEQ ID NO: 29.

In some embodiments, at least one RNA comprises a poly-A tail. In some embodiments, each RNA comprises a poly-A tail. In some embodiments, the poly-A tail may comprise at least 20, at least 30, at least 40, at least 80, or at least 100 and up to 500, up to 400, up to 300, up to 200, or up to 150 nucleotides. In some embodiments, the poly-A tail may essentially consist of at least 20, at least 30, at least 40, at least 80, or at least 100 and up to 500, up to 400, up to 300, up to 200, or up to 150 nucleotides. In some embodiments, the poly-A tail may consist of at least 20, at least 30, at least 40, at least 80, or at least 100 and up to 500, up to 400, up to 300, up to 200, or up to 150 nucleotides. In some embodiments, the poly-A tail may comprise the poly-A tail shown in SEQ ID NO: 29. In some embodiments, the poly-A tail comprises at least 100 nucleotides. In some embodiments, the poly-A tail comprises about 150 nucleotides. In some embodiments, the poly-A tail comprises about 120 nucleotides.

In the context of the present disclosure, the term “transcription” relates to a process, wherein the genetic code in a DNA sequence is transcribed into RNA. Subsequently, the RNA may be translated into peptide or protein.

With respect to RNA, the term “expression” or “translation” relates to the process in the ribosomes of a cell by which a strand of mRNA directs the assembly of a sequence of amino acids to make a peptide or protein.

In one embodiment, after administration of the RNA described herein, e.g., formulated as RNA lipoplex particles, at least a portion of the RNA is delivered to a target cell. In one embodiment, at least a portion of the RNA is delivered to the cytosol of the target cell. In one embodiment, the RNA is translated by the target cell to produce the peptide or protein it enodes. In one embodiment, the target cell is a spleen cell. In one embodiment, the target cell is an antigen presenting cell such as a professional antigen presenting cell in the spleen. In one embodiment, the target cell is a dendritic cell or macrophage. RNA lipoplex particles described herein may be used for delivering RNA to such target cell. Accordingly, the present disclosure also relates to a method for delivering RNA to a target cell in a subject comprising the administration of the RNA lipoplex particles described herein to the subject. In one embodiment, the RNA is delivered to the cytosol of the target cell. In one embodiment, the RNA is translated by the target cell to produce the peptide or protein encoded by the RNA. According to the disclosure, the term “RNA encodes” means that the RNA, if present in the appropriate environment, such as within cells of a target tissue, can direct the assembly of amino acids to produce the peptide or protein it encodes during the process of translation. In one embodiment, RNA is able to interact with the cellular translation machinery allowing translation of the peptide or protein. A cell may produce the encoded peptide or protein intracellularly (e.g., in the cytoplasm and/or in the nucleus), may secrete the encoded peptide or protein, or may produce it on the surface.

According to the disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” refers to large peptides, in particular peptides having at least about 151 amino acids, but the terms “peptide” and “protein” are used herein usually as synonyms.

The term “antigen” relates to an agent comprising an epitope against which an immune response can be generated. The term “antigen” includes, in particular, proteins and peptides. In one embodiment, an antigen is presented by cells of the immune system such as antigen presenting cells like dendritic cells or macrophages. An antigen or a processing product thereof such as a T-cell epitope is in one embodiment bound by a T- or B-cell receptor, or by an immunoglobulin molecule such as an antibody. Accordingly, an antigen or a processing product thereof may react specifically with antibodies or T lymphocytes (T cells). In one embodiment, an antigen is a disease-associated antigen, such as a tumor antigen and an epitope is derived from such antigen.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. The disease-associated antigen or an epitope thereof may therefore be used for therapeutic purposes. Disease-associated antigens may be associated with cancer, typically tumors.

The term “tumor antigen” refers to a constituent of cancer cells which may be derived from the cytoplasm, the cell surface and the cell nucleus. In particular, it refers to those antigens which are produced intracellularly or as surface antigens on tumor cells.

The term “epitope” refers to a part or fragment a molecule such as an antigen that is recognized by the immune system. For example, the epitope may be recognized by T cells, B cells or antibodies. An epitope of an antigen may include a continuous or discontinuous portion of the antigen and may be between about 5 and about 100 amino acids in length. In one embodiment, an epitope is between about 10 and about 25 amino acids in length. The term “epitope” includes T-cell epitopes.

The term “T-cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T-cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

In certain embodiments of the present disclosure, the RNA encodes at least one epitope. In certain embodiments, the epitope is derived from a tumor antigen as described herein.

Administered RNAs

In some embodiments, compositions described herein comprise RNA encoding a Kallikrein-2 (KLK2) protein, RNA encoding a Prostate Specific Antigen (PSA) protein, RNA encoding a Prostatic Acid Phosphatase (PAP) protein, RNA encoding a Homeobox B13 (HOXB13) protein, and RNA encoding a NK3 Homeobox 1 (NKX3-1) protein. Likewise, methods described herein comprise administration of RNA encoding a Kallikrein-2 (KLK2) protein, RNA encoding a Prostate Specific Antigen (PSA) protein, RNA encoding a Prostatic Acid Phosphatase (PAP) protein, RNA encoding a Homeobox B13 (HOXB13) protein, and RNA encoding a NK3 Homeobox 1 (NKX3-1) protein.

A Kallikrein-2 (KLK2) protein comprises an amino acid sequence comprising KLK2, an immunogenic variant thereof, or an immunogenic fragment of the KLK2 or the immunogenic variant thereof, and may have an amino acid sequence comprising the amino acid sequence of SEQ ID NO: 1 or 2, or an amino acid sequence having at least 99%, 98%, 97%, 96%, 95%, 90%, 85%, or 80% identity to the amino acid sequence of SEQ ID NO:1 or 2. RNA encoding a KLK2 protein (i) may comprise the nucleotide sequence of SEQ ID NO: 3 or 4, or a nucleotide sequence having at least 99%, 98%, 97%, 96%, 95%, 90%, 85%, or 80% identity to the nucleotide sequence of SEQ ID NO: 3 or 4; and/or (ii) may encode an amino acid sequence comprising the amino acid sequence of SEQ ID NO: 1 or 2, or an amino acid sequence having at least 99%, 98%, 97%, 96%, 95%, 90%, 85%, or 80% identity to the amino acid sequence of SEQ ID NO: 1 or 2.

A Prostate Specific Antigen (PSA) protein comprises an amino acid sequence comprising PSA, an immunogenic variant thereof, or an immunogenic fragment of the PSA or the immunogenic variant thereof, and may have an amino acid sequence comprising the amino acid sequence of SEQ ID NO: 5 or 6, or an amino acid sequence having at least 99%, 98%, 97%, 96%, 95%, 90%, 85%, or 80% identity to the amino acid sequence of SEQ ID NO: 5 or 6. RNA encoding a PSA protein (i) may comprise the nucleotide sequence of SEQ ID NO: 7 or 8, or a nucleotide sequence having at least 99%, 98%, 97%, 96%, 95%, 90%, 85%, or 80% identity to the nucleotide sequence of SEQ ID NO: 7 or 8; and/or (ii) may encode an amino acid sequence comprising the amino acid sequence of SEQ ID NO: 5 or 6, or an amino acid sequence having at least 99%, 98%, 97%, 96%, 95%, 90%, 85%, or 80% identity to the amino acid sequence of SEQ ID NO: 5 or 6.

A Prostatic Acid Phosphatase (PAP) protein comprises an amino acid sequence comprising PAP, an immunogenic variant thereof, or an immunogenic fragment of the PAP or the immunogenic variant thereof, and may have an amino acid sequence comprising the amino acid sequence of SEQ ID NO: 9 or 10, or an amino acid sequence having at least 99%, 98%, 97%, 96%, 95%, 90%, 85%, or 80% identity to the amino acid sequence of SEQ ID NO: 9 or 10. RNA encoding a PAP protein (i) may comprise the nucleotide sequence of SEQ ID NO: 11 or 12, or a nucleotide sequence having at least 99%, 98%, 97%, 96%, 95%, 90%, 85%, or 80% identity to the nucleotide sequence of SEQ ID NO: 11 or 12; and/or (ii) may encode an amino acid sequence comprising the amino acid sequence of SEQ ID NO: 9 or 10, or an amino acid sequence having at least 99%, 98%, 97%, 96%, 95%, 90%, 85%, or 80% identity to the amino acid sequence of SEQ ID NO: 9 or 10.

A Homeobox B13 (HOXB13) protein comprises an amino acid sequence comprising HOXB13, an immunogenic variant thereof, or an immunogenic fragment of the HOXB13 or the immunogenic variant thereof, and may have an amino acid sequence comprising the amino acid sequence of SEQ ID NO: 13 or 14, or an amino acid sequence having at least 99%, 98%, 97%, 96%, 95%, 90%, 85%, or 80% identity to the amino acid sequence of SEQ ID NO: 13 or 14. RNA encoding a HOXB13 protein (i) may comprise the nucleotide sequence of SEQ ID NO: 15 or 16, or a nucleotide sequence having at least 99%, 98%, 97%, 96%, 95%, 90%, 85%, or 80% identity to the nucleotide sequence of SEQ ID NO: 15 or 16; and/or (ii) may encode an amino acid sequence comprising the amino acid sequence of SEQ ID NO: 13 or 14, or an amino acid sequence having at least 99%, 98%, 97%, 96%, 95%, 90%, 85%, or 80% identity to the amino acid sequence of SEQ ID NO: 13 or 14.

A NK3 Homeobox 1 (NKX3-1) protein comprises an amino acid sequence comprising NKX3-1, an immunogenic variant thereof, or an immunogenic fragment of the NKX3-1 or the immunogenic variant thereof, and may have an amino acid sequence comprising the amino acid sequence of SEQ ID NO: 17 or 18, or an amino acid sequence having at least 99%, 98%, 97%, 96%, 95%, 90%, 85%, or 80% identity to the amino acid sequence of SEQ ID NO: 17 or 18. RNA encoding a NKX3-1 protein (i) may comprise the nucleotide sequence of SEQ ID NO: 19 or 20, or a nucleotide sequence having at least 99%, 98%, 97%, 96%, 95%, 90%, 85%, or 80% identity to the nucleotide sequence of SEQ ID NO: 19 or 20; and/or (ii) may encode an amino acid sequence comprising the amino acid sequence of SEQ ID NO: 17 or 18, or an amino acid sequence having at least 99%, 98%, 97%, 96%, 95%, 90%, 85%, or 80% identity to the amino acid sequence of SEQ ID NO: 17 or 18.

By “variant” herein is meant an amino acid sequence that differs from a parent amino acid sequence by virtue of at least one amino acid modification. The parent amino acid sequence may be a naturally occurring or wild type (WT) amino acid sequence, or may be a modified version of a wild type amino acid sequence. Preferably, the variant amino acid sequence has at least one amino acid modification compared to the parent amino acid sequence, e.g., from 1 to about 20 amino acid modifications, and preferably from 1 to about 10 or from 1 to about 5 amino acid modifications compared to the parent.

By “wild type” or “WT” or “native” herein is meant an amino acid sequence that is found in nature, including allelic variations. A wild type amino acid sequence, peptide or protein has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:

glycine, alanine;

valine, isoleucine, leucine;

aspartic acid, glutamic acid;

asparagine, glutamine;

serine, threonine;

lysine, arginine; and

phenylalanine, tyrosine.

Preferably the degree of similarity, preferably identity between a given amino acid sequence and an amino acid sequence which is a variant of said given amino acid sequence will be at least about 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 100, at least about 120, at least about 140, at least about 160, at least about 180, or about 200 amino acids, preferably continuous amino acids. In preferred embodiments, the degree of similarity or identity is given for the entire length of the reference amino acid sequence.

“Sequence similarity” indicates the percentage of amino acids that either are identical or that represent conservative amino acid substitutions. “Sequence identity” between two amino acid sequences indicates the percentage of amino acids that are identical between the sequences.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof.

A peptide and protein antigen described herein (KLK2 protein, PSA protein, PAP protein, HOXB13 protein, and NKX3-1 protein) when provided to a subject by administration of RNA encoding the antigen, i.e., a vaccine antigen, preferably results in stimulation, priming and/or expansion of T cells in the subject. Said stimulated, primed and/or expanded T cells are preferably directed against the target antigen, in particular the target antigen expressed by diseased cells, tissues and/or organs, i.e., the disease-associated antigen. Thus, a vaccine antigen may comprise the disease-associated antigen, or a fragment or variant thereof. In one embodiment, such fragment or variant is immunologically equivalent to the disease-associated antigen. In the context of the present disclosure, the term “fragment of an antigen” or “variant of an antigen” means an agent which results in stimulation, priming and/or expansion of T cells which stimulated, primed and/or expanded T cells target the disease-associated antigen, in particular when expressed on the surface of diseased cells, tissues and/or organs. Thus, the vaccine antigen administered according to the disclosure may correspond to or may comprise the disease-associated antigen, may correspond to or may comprise a fragment of the disease-associated antigen or may correspond to or may comprise an antigen which is homologous to the disease-associated antigen or a fragment thereof. If the vaccine antigen administered according to the disclosure comprises a fragment of the disease-associated antigen or an amino acid sequence which is homologous to a fragment of the disease-associated antigen said fragment or amino acid sequence may comprise an epitope of the disease-associated antigen or a sequence which is homologous to an epitope of the disease-associated antigen, wherein the T cells bind to said epitope. Thus, according to the disclosure, an antigen may comprise an immunogenic fragment of the disease-associated antigen or an amino acid sequence being homologous to an immunogenic fragment of the disease-associated antigen. An “immunogenic fragment of an antigen” according to the disclosure preferably relates to a fragment of an antigen which is capable of stimulating, priming and/or expanding T cells. It is preferred that the vaccine antigen (similar to the disease-associated antigen) provides the relevant epitope for binding by T cells. It is also preferred that the vaccine antigen (similar to the disease-associated antigen) is expressed on the surface of a cell such as an antigen-presenting cell so as to provide the relevant epitope for binding by the T cells. The vaccine antigen according to the invention may be a recombinant antigen.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants. For example, an amino acid sequence is immunologically equivalent to a reference amino acid sequence, if said amino acid sequence when exposed toT cells binding to the reference amino acid sequence or cells expressing the reference amino acid sequence induces an immune reaction having a specificity of reacting with the reference amino acid sequence, in particular stimulation, priming and/or expansion of T cells. Thus, a molecule which is immunologically equivalent to an antigen exhibits the same or essentially the same properties and/or exerts the same or essentially the same effects regarding the stimulation, priming and/or expansion of T cells as the antigen to which the T cells are targeted.

“Activation” or “stimulation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division. The term “priming” refers to a process wherein a T cell has its first contact with its specific antigen and causes differentiation into effector T cells.

The term “clonal expansion” or “expansion” refers to a process wherein a specific entity is multiplied. In the context of the present disclosure, the term is preferably used in the context of an immunological response in which lymphocytes are stimulated by an antigen, proliferate, and the specific lymphocyte recognizing said antigen is amplified. Preferably, clonal expansion leads to differentiation of the lymphocytes.

Lipoplex Particles

In certain embodiments of the present disclosure, the RNA described herein may be present in RNA lipoplex particles. The RNA lipoplex particles and compositions comprising RNA lipoplex particles described herein are useful for delivery of RNA to a target tissue after parenteral administration, in particular after intravenous administration. The RNA lipoplex particles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE). Liposomes may be used for preparing RNA lipoplex particles by mixing the liposomes with RNA.

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

RNA Lipoplex Particle Diameter

RNA lipoplex particles described herein have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

In one embodiment, RNA lipoplex particles described herein exhibit a polydispersity index less than about 0.5, less than about 0.4, or less than about 0.3. By way of example, the RNA lipoplex particles can exhibit a polydispersity index in a range of about 0.1 to about 0.3.

Lipid

In one embodiment, the lipid solutions, liposomes and RNA lipoplex particles described herein include a cationic lipid. As used herein, a “cationic lipid” refers to a lipid having a net positive charge. Cationic lipids bind negatively charged RNA by electrostatic interaction to the lipid matrix. Generally, cationic lipids possess a lipophilic moiety, such as a sterol, an acyl or diacyl chain, and the head group of the lipid typically carries the positive charge. Examples of cationic lipids include, but are not limited to 1,2-di-O-octadecenyl-3-trimethylammonium propane (DOTMA), dimethyldioctadecylammonium (DDAB); 1,2-dioleoyl-3-trimethylammonium propane (DOTAP); 1,2-dioleoyl-3-dimethylammonium-propane (DODAP); 1,2-diacyloxy-3-dimethylammonium propanes; 1,2-dialkyloxy-3- dimethylammonium propanes; dioctadecyldimethyl ammonium chloride (DODAC), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA). Preferred are DOTMA, DOTAP, DODAC, and DOSPA. In specific embodiments, the cationic lipid is DOTMA and/or DOTAP.

An additional lipid may be incorporated to adjust the overall positive to negative charge ratio and physical stability of the RNA lipoplex particles. In certain embodiments, the additional lipid is a neutral lipid. As used herein, a “neutral lipid” refers to a lipid having a net charge of zero. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, cholesterol, and cerebroside. In specific embodiments, the additional lipid is DOPE, cholesterol and/or DOPC.

In certain embodiments, the RNA lipoplex particles include both a cationic lipid and an additional lipid. In an exemplary embodiment, the cationic lipid is DOTMA and the additional lipid is DOPE. Without wishing to be bound by theory, the amount of the at least one cationic lipid compared to the amount of the at least one additional lipid may affect important RNA lipoplex particle characteristics, such as charge, particle size, stability, tissue selectivity, and bioactivity of the RNA. Accordingly, in some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

Charge Ratio

The electric charge of the RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)]. The concentration of RNA and the at least one cationic lipid amount can be determined using routine methods by one skilled in the art. In one embodiment, at physiological pH the charge ratio of positive charges to negative charges in the RNA lipoplex particles is from about 1.6:2 to about 1:2, or about 1.6:2 to about 1.1:2. In specific embodiments, the charge ratio of positive charges to negative charges in the RNA lipoplex particles at physiological pH is about 1.6:2.0, about 1.5:2.0, about 1.4:2.0, about 1.3:2.0, about 1.2:2.0, about 1.1:2.0, or about 1:2.0.

It has been found that RNA lipoplex particles having such charge ratio may be used to preferentially target spleen tissue or spleen cells such as antigen-presenting cells, in particular dendritic cells. Accordingly, in one embodiment, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

A. Salt and Ionic Strength

According to the present disclosure, the compositions described herein may comprise salts such as sodium chloride. Without wishing to be bound by theory, sodium chloride functions as an ionic osmolality agent for preconditioning RNA prior to mixing with the at least one cationic lipid. Certain embodiments contemplate alternative organic or inorganic salts to sodium chloride in the present disclosure. Alternative salts include, without limitation, potassium chloride, dipotassium phosphate, monopotassium phosphate, potassium acetate, potassium bicarbonate, potassium sulfate, potassium acetate, disodium phosphate, monosodium phosphate, sodium acetate, sodium bicarbonate, sodium sulfate, sodium acetate, lithium chloride, magnesium chloride, magnesium phosphate, calcium chloride, and sodium salts of ethylenediaminetetraacetic acid (EDTA).

Generally, compositions comprising RNA lipoplex particles described herein comprise sodium chloride at a concentration that preferably ranges from 0 mM to about 500 mM, from about 5 mM to about 400 mM, or from about 10 mM to about 300 mM. In one embodiment, compositions comprising RNA lipoplex particles comprise an ionic strength corresponding to such sodium chloride concentrations.

B. Stabilizer

Compositions described herein may comprise a stabilizer to avoid substantial loss of the product quality and, in particular, substantial loss of RNA activity during freezing, lyophilization, spray-drying or storage such as storage of the frozen, lyophilized or spray-dried composition.

In an embodiment the stabilizer is a carbohydrate. The term “carbohydrate”, as used herein refers to and encompasses monosaccharides, disaccharides, trisaccharides, oligosaccharides, and polysaccharides.

In embodiments of the disclosure, the stabilizer is mannose, glucose, sucrose or trehalose. According to the present disclosure, the RNA lipoplex particle compositions described herein have a stabilizer concentration suitable for the stability of the composition, in particular for the stability of the RNA lipoplex particles and for the stability of the RNA.

C. pH and Buffer

According to the present disclosure, the RNA lipoplex particle compositions described herein have a pH suitable for the stability of the RNA lipoplex particles and, in particular, for the stability of the RNA. In one embodiment, the RNA lipoplex particle compositions described herein have a pH from about 5.5 to about 7.5.

According to the present disclosure, compositions that include buffer are provided. Without wishing to be bound by theory, the use of buffer maintains the pH of the composition during manufacturing, storage and use of the composition. In certain embodiments of the present disclosure, the buffer may be sodium bicarbonate, monosodium phosphate, disodium phosphate, monopotassium phosphate, dipotassium phosphate, [tris(hydroxymethyl)methylamino]propanesulfonic acid (TAPS), 2-(Bis(2-hydroxyethyl)amino)acetic acid (Bicine), 2-Amino-2-(hydroxymethyl)propane-1,3-diol (Tris), N-(2-Hydroxy-1,1-bis(hydroxymethyl)ethyl)glycine (Tricine), 3-[[1,3-dihydroxy-2-(hydroxymethyl)propan-2-yl]amino]-2-hydroxypropane-1-sulfonic acid (TAPSO), 2-[4-(2-hydroxyethyl)piperazin-1-yl]ethanesulfonic acid (HEPES), 2-[[1,3-dihydroxy-2-(hydroxymethyl)propan-2-yl]amino]ethanesulfonic acid (TES), 1,4-piperazinediethanesulfonic acid (PIPES), dimethylarsinic acid, 2-morpholin-4-ylethanesulfonic acid (MES), 3-morpholino-2-hydroxypropanesulfonic acid (MOPSO), or phosphate buffered saline (PBS). Other suitable buffers may be acetic acid in a salt, citric acid in a salt, boric acid in a salt and phosphoric acid in a salt.

In one embodiment, the buffer is HEPES.

In one embodiment, the buffer has a concentration from about 2.5 mM to about 15 mM.

D. Chelating Agent

Certain embodiments of the present disclosure contemplate the use of a chelating agent. Chelating agents refer to chemical compounds that are capable of forming at least two coordinate covalent bonds with a metal ion, thereby generating a stable, water-soluble complex. Without wishing to be bound by theory, chelating agents reduce the concentration of free divalent ions, which may otherwise induce accelerated RNA degradation in the present disclosure. Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid (DTPA), bis(aminoethyl)glycolether-N,N,N′,N′-tetraacetic acid, iminodiacetic acid, citric acid, tartaric acid, fumaric acid, or a salt thereof. In certain embodiments, the chelating agent is EDTA or a salt of EDTA. In an exemplary embodiment, the chelating agent is EDTA disodium dihydrate.

In some embodiments, the EDTA is at a concentration from about 0.05 mM to about 5 mM.

E. Physical State of Compositions of the Disclosure

In embodiments, the composition of the present disclosure is a liquid or a solid. Non-limiting examples of a solid include a frozen form or a lyophilized form. In a preferred embodiment, the composition is a liquid.

Pharmaceutical Compositions of the Disclosure

The RNA described herein, e.g., formulated as RNA lipoplex particles, is useful as or for preparing pharmaceutical compositions or medicaments for therapeutic or prophylactic treatments.

The compositions of the present disclosure may be administered in the form of any suitable pharmaceutical composition.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation. In the context of the present disclosure, the pharmaceutical composition comprises the RNA described herein, e.g., formulated as RNA lipoplex particles. The pharmaceutical compositions of the present disclosure preferably comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cyctokines, such as monokines, lymphokines, interleukins, chemokines. The chemokines may be IL-1, IL-2, IL-3, IL-4, IL-5, IL-6, IL-7, IL-8, IL-9, IL-10, IL-12, INFa, INF-γ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys. The pharmaceutical compositions according to the present disclosure are generally applied in a “pharmaceutically effective amount” and in “a pharmaceutically acceptable preparation”. The term “pharmaceutically acceptable” refers to the non-toxicity of a material which does not interact with the action of the active component of the pharmaceutical composition. The term “pharmaceutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

In some embodiments, an effective amount comprises an amount sufficient to cause a tumor/lesion to shrink. In some embodiments, an effective amount is an amount sufficient to decrease the growth rate of a tumor (such as to suppress tumor growth). In some embodiments, an effective amount is an amount sufficient to delay tumor development. In some embodiments, an effective amount is an amount sufficient to prevent or delay tumor recurrence. In some embodiments, an effective amount is an amount sufficient to increase a subject's immune response to a tumor, such that tumor growth and/or size and/or metastasis is reduced, delayed, ameliorated, and/or prevented. An effective amount can be administered in one or more administrations. In some embodiments, administration of an effective amount (e.g., of a composition comprising mRNAs) may: (i) reduce the number of cancer cells; (ii) reduce tumor size; (iii) inhibit, retard, slow to some extent and may stop cancer cell infiltration into peripheral organs; (iv) inhibit (e.g., slow to some extent and/or block or prevent) metastasis; (v) inhibit tumor growth; (vi) prevent or delay occurrence and/or recurrence of tumor; and/or (vii) relieve to some extent one or more of the symptoms associated with the cancer.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents, and/or excipients.

Suitable preservatives for use in the pharmaceutical compositions of the present disclosure include, without limitation, benzalkonium chloride, chlorobutanol, paraben, and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid, or solid suspension and/or mixing media. Examples of suitable diluents include ethanol, glycerol, and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents orencapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients, or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

Routes of Administration of Pharmaceutical Compositions of the Disclosure

In one embodiment, pharmaceutical compositions described herein may be administered intravenously, intraarterially, subcutaneously, intradermally, intranodullary or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical composition is formulated for systemic administration. In another preferred embodiment, the systemic administration is by intravenous administration.

Use of Pharmaceutical Compositions of the Disclosure

The RNA described herein, e.g., formulated as RNA lipoplex particles, may be used in the therapeutic or prophylactic treatment of diseases in which provision of amino acid sequences encoded by the RNA to a subject results in a therapeutic or prophylactic effect. The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating”, or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse, or primate) that can be afflicted with or is susceptible to a disease or disorder (e.g., cancer) but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In one embodiment of the disclosure, the aim is to provide an immune response against cancer cells expressing one or more tumor antigens, and to treat a cancer disease involving cells expressing one or more tumor antigens. In one embodiment, the cancer is prostate cancer. In one embodiment, the tumor antigens are KLK2, PSA, PAP, HOXB13, and/or NKX3-1. A pharmaceutical composition comprising RNA may be administered to a subject to elicit an immune response against one or more antigens or one or more epitopes encoded by the RNA in the subject which may be therapeutic or partially or fully protective. A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, pharmaceutical compositions described herein are applicable for inducing or enhancing an immune response. Pharmaceutical compositions described herein are thus useful in a prophylactic and/or therapeutic treatment of a disease involving an antigen or epitope, in particular prostate cancer.

As used herein, “immune response” refers to an integrated bodily response to an antigen or a cell expressing an antigen and refers to a cellular immune response and/or a humoral immune response. A cellular immune response includes, without limitation, a cellular response directed to cells expressing an antigen and being characterized by presentation of an antigen with class I or class II MHC molecule. The cellular response relates to T lymphocytes, which may be classified as helper T cells (also termed CD4+ T cells) that play a central role by regulating the immune response or killer cells (also termed cytotoxic T cells, CD8+ T cells, or CTLs) that induce apoptosis in infected cells or cancer cells. In one embodiment, administering a pharmaceutical composition of the present disclosure involves stimulation of an anti-tumor CD8+ T-cell response against cancer cells expressing one or more tumor antigens. In a specific embodiment, the tumor antigens are presented with class I MHC molecule.

The present disclosure contemplates an immune response that may be protective, preventive, prophylactic, and/or therapeutic. As used herein, “induces [or inducing] an immune response” may indicate that no immune response against a particular antigen was present before induction or it may indicate that there was a basal level of immune response against a particular antigen before induction, which was enhanced after induction. Therefore, “induces [or inducing] an immune response” includes “enhances [or enhancing] an immune response”. The term “immunotherapy” relates to the treatment of a disease or condition by inducing, or enhancing an immune response. The term “immunotherapy” includes antigen immunization or antigen vaccination.

The terms “immunization” or “vaccination” describe the process of administering an antigen to an individual with the purpose of inducing an immune response, for example, for therapeutic or prophylactic reasons.

In one embodiment, the present disclosure envisions embodiments wherein RNA lipoplex particles as described herein targeting spleen tissue are administered. The RNA encodes a peptide or protein comprising an antigen or an epitope as described, for example, herein. The RNA is taken up by antigen-presenting cells in the spleen such as dendritic cells to express the peptide or protein. Following optional processing and presentation by the antigen-presenting cells an immune response may be generated against the antigen or epitope resulting in a prophylactic and/or therapeutic treatment of a disease involving the antigen or epitope. In one embodiment, the immune response induced by the RNA lipoplex particles described herein comprises presentation of an antigen or fragment thereof, such as an epitope, by antigen presenting cells, such as dendritic cells and/or macrophages, and activation of cytotoxic T cells due to this presentation. For example, peptides or proteins encoded by the RNAs or procession products thereof may be presented by major histocompatibility complex (MHC) proteins expressed on antigen presenting cells. The MHC peptide complex can then be recognized by immune cells such as T cells or B cells leading to their activation.

Thus, in one embodiment the RNA in the RNA lipoplex particles described herein, following administration, is delivered to the spleen and/or is expressed in the spleen. In one embodiment, the RNA lipoplex particles are delivered to the spleen for activating splenic antigen presenting cells. Thus, in one embodiment, after administration of the RNA lipoplex particles RNA delivery and/or RNA expression in antigen presenting cells occurs. Antigen presenting cells may be professional antigen presenting cells or non-professional antigen presenting cells. The professional antigen presenting cells may be dendritic cells and/or macrophages, even more preferably splenic dendritic cells and/or splenic macrophages.

Accordingly, the present disclosure relates to RNA lipoplex particles or a pharmaceutical composition comprising RNA lipoplex particles as described herein for inducing or enhancing an immune response, preferably an immune response against prostate cancer.

In one embodiment, systemically administering RNA lipoplex particles or a pharmaceutical composition comprising RNA lipoplex particles as described herein results in targeting and/or accumulation of the RNA lipoplex particles or RNA in the spleen and not in the lung and/or liver. In one embodiment, RNA lipoplex particles release RNA in the spleen and/or enter cells in the spleen. In one embodiment, systemically administering RNA lipoplex particles or a pharmaceutical composition comprising RNA lipoplex particles as described herein delivers the RNA to antigen presenting cells in the spleen. In a specific embodiment, the antigen presenting cells in the spleen are dendritic cells or macrophages.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B-cell surface, resulting in T- and B-cell activation and further stimulation of the immune response. Macrophages belong to the class of antigen presenting cells. In one embodiment, the macrophages are splenic macrophages.

The term “dendritic cell” (DC) refers to another subtype of phagocytic cells belonging to the class of antigen presenting cells. In one embodiment, dendritic cells are derived from hematopoietic bone marrow progenitor cells. These progenitor cells initially transform into immature dendritic cells. These immature cells are characterized by high phagocytic activity and low T-cell activation potential. Immature dendritic cells constantly sample the surrounding environment for pathogens such as viruses and bacteria. Once they have come into contact with a presentable antigen, they become activated into mature dendritic cells and begin to migrate to the spleen orto the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T-cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helperT cells and killerT cells as well as B cells by presenting them antigens, alongside non-antigen specific co-stimulatory signals. Thus, dendritic cells can actively induce a T-cell- or B-cell-related immune response. In one embodiment, the dendritic cells are splenic dendritic cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naïve T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

The term “disease involving an antigen” or “disease involving an epitope” refers to any disease which implicates an antigen or epitope, e.g., a disease which is characterized by the presence of an antigen or epitope. The disease involving an antigen or epitope can be a cancer disease or simply cancer. As mentioned above, the antigen may be a disease-associated antigen, such as a tumor-associated antigen and the epitope may be derived from such antigen.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. One particular form of cancer that can be treated by the compositions and methods described herein is prostate cancer. The term “cancer” according to the disclosure also comprises cancer metastases.

Combination strategies in cancer treatment may be desirable due to a resulting synergistic effect, which may be considerably stronger than the impact of a monotherapeutic approach. In one embodiment, the pharmaceutical composition is administered with an immunotherapeutic agent. As used herein “immunotherapeutic agent” relates to any agent that may be involved in activating a specific immune response and/or immune effector function(s). The present disclosure contemplates the use of an antibody as an immunotherapeutic agent. Without wishing to be bound by theory, antibodies are capable of achieving a therapeutic effect against cancer cells through various mechanisms, including inducing apoptosis, block components of signal transduction pathways or inhibiting proliferation of tumor cells. In certain embodiments, the antibody is a monoclonal antibody. A monoclonal antibody may induce cell death via antibody-dependent cell mediated cytotoxicity (ADCC), or bind complement proteins, leading to direct cell toxicity, known as complement dependent cytotoxicity (CDC). Non-limiting examples of anti-cancer antibodies and potential antibody targets (in brackets) which may be used in combination with the present disclosure include: Abagovomab (CA-125), Abciximab (CD41), Adecatumumab (EpCAM), Afutuzumab (CD20), Alacizumab pegol (VEGFR2), Altumomab pentetate (CEA), Amatuximab (MORAb-009), Anatumomab mafenatox (TAG-72), Apolizumab (HLA-DR), Arcitumomab (CEA), Atezolizumab (PD-L1), Bavituximab (phosphatidylserine), Bectumomab (CD22), Belimumab (BAFF), Bevacizumab (VEGF-A), Bivatuzumab mertansine (CD44 v6), Blinatumomab (CD 19), Brentuximab vedotin (CD30 TNFRSF8), Cantuzumab mertansin (mucin CanAg), Cantuzumab ravtansine (MUC1), Capromab pendetide (prostatic carcinoma cells), Carlumab (CNT0888), Catumaxomab (EpCAM, CD3), Cetuximab (EGFR), Citatuzumab bogatox (EpCAM), Cixutumumab (IGF-1 receptor), Claudiximab (Claudin), Clivatuzumab tetraxetan (MUC1), Conatumumab (TRAIL-R2), Dacetuzumab (CD40), Dalotuzumab (insulin-like growth factor I receptor), Denosumab (RANKL), Detumomab (B-lymphoma cell), Drozitumab (DR5), Ecromeximab (GD3 ganglioside), Edrecolomab (EpCAM), Elotuzumab (SLAMF7), Enavatuzumab (PDL192), Ensituximab (NPC-1C), Epratuzumab (CD22), Ertumaxomab (HER2/neu, CD3), Etaracizumab (integrin av33), Farletuzumab (folate receptor 1), FBTA05 (CD20), Ficlatuzumab (SCH 900105), Figitumumab (IGF-1 receptor), Flanvotumab (glycoprotein 75), Fresolimumab (TGF-3), Galiximab (CD80), Ganitumab (IGF-I), Gemtuzumab ozogamicin (CD33), Gevokizumab (IL-I3), Girentuximab (carbonic anhydrase 9 (CA-IX)), Glembatumumab vedotin (GPNMB), Ibritumomab tiuxetan (CD20), Icrucumab (VEGFR-1), Igovoma (CA-125), Indatuximab ravtansine (SDC1), Intetumumab (CD51), Inotuzumab ozogamicin (CD22), Ipilimumab (CD 152), Iratumumab (CD30), Labetuzumab (CEA), Lexatumumab (TRAIL-R2), Libivirumab (hepatitis B surface antigen), Lintuzumab (CD33), Lorvotuzumab mertansine (CD56), Lucatumumab (CD40), Lumiliximab (CD23), Mapatumumab (TRAIL-R1), Matuzumab (EGFR), Mepolizumab (IL-5), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Namatumab (RON), Necitumumab (EGFR), Nimotuzumab (EGFR), Nivolumab (IgG4), Ofatumumab (CD20), Olaratumab (PDGF-R a), Onartuzumab (human scatter factor receptor kinase), Oportuzumab monatox (EpCAM), Oregovomab (CA-125), Oxelumab (OX-40), Panitumumab (EGFR), Patritumab (HER3), Pemtumoma (MUC1), Pertuzuma (HER2/neu), Pintumomab (adenocarcinoma antigen), Pritumumab (vimentin), Racotumomab (N-glycolylneuraminic acid), Radretumab (fibronectin extra domain-B), Rafivirumab (rabies virus glycoprotein), Ramucirumab (VEGFR2), Rilotumumab (HGF), Rituximab (CD20), Robatumumab (IGF-1 receptor), Samalizumab (CD200), Sibrotuzumab (FAP), Siltuximab (IL-6), Tabalumab (BAFF), Tacatuzumab tetraxetan (alpha-fetoprotein), Taplitumomab paptox (CD 19), Tenatumomab (tenascin C), Teprotumumab (CD221), Ticilimumab (CTLA-4), Tigatuzumab (TRAIL-R2), TNX-650 (IL-13), Tositumomab (CD20), Trastuzumab (HER2/neu), TRBSO7 (GD2), Tremelimumab (CTLA-4), Tucotuzumab celmoleukin (EpCAM), Ublituximab (MS4A1), Urelumab (4-1 BB), Volociximab (integrin a531), Votumumab (tumor antigen CTAA 16.88), Zalutumumab (EGFR), and Zanolimumab (CD4).

In one embodiment, the immunotherapeutic agent is a PD-1 axis binding antagonist. A PD-1 axis binding antagonist includes but is not limited to a PD-1 binding antagonist, a PD-L1 binding antagonist and a PD-L2 binding antagonist. Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, the PD-1 binding antagonist is a molecule that inhibits the binding of PD-1 to its ligand binding partners. In a specific aspect the PD-1 ligand binding partners are PD-L1 and/or PD-L2. In another embodiment, a PD-L1 binding antagonist is a molecule that inhibits the binding of PD-L1 to its binding partners. In a specific embodiment, PD-L1 binding partners are PD-1 and/or B7- 1. In another embodiment, the PD-L2 binding antagonist is a molecule that inhibits the binding of PD-L2 to its binding partners. In a specific embodiment, the PD-L2 binding partner is PD-1. The PD-1 binding antagonist may be an antibody, an antigen binding fragment thereof, an immunoadhesin, a fusion protein, or oligopeptide. In some embodiments, the PD-1 binding antagonist is an anti-PD-1 antibody (e.g., a human antibody, a humanized antibody, or a chimeric antibody). Examples of an anti-PD-1 antibody include, without limitation, MDX-1106 (Nivolumab, OPDIVO), Merck 3475 (MK-3475, Pembrolizumab, KEYTRUDA), MEDI-0680 (AMP-514), PDR001, REGN2810, BGB-108, and BGB-A317.

In one embodiment, the PD-1 binding antagonist is an immunoadhesin that includes an extracellular or PD-1 binding portion of PD-L1 or PD-L2 fused to a constant region. In one embodiment, the PD-1 binding antagonist is AMP-224 (also known as B7-DCIg, is a PD-L2- Fc), which is fusion soluble receptor described in WO2010/027827 and WO201 1/066342. In one embodiment, the PD-1 binding antagonist is an anti-PD-L1 antibody, including, without limitation, YW243.55.S70, MPDL3280A (Atezolizumab), MEDI4736 (Durvalumab), MDX-1105, and MSB0010718C (Avelumab).

In one embodiment, the immunotherapeutic agent is a PD-1 binding antagonist. In another embodiment, the PD-1 binding antagonist is an anti-PD-L1 antibody. In an exemplary embodiment, the anti-PD-L1 antibody is Atezolizumab.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Intravenous Vaccine for Treating Prostate Cancer

Described herein is a second generation vaccine for intravenous (IV) application. It consists of RNAs targeting five antigens expressed in prostate cancer that are separately complexed with liposomes to serum-stable RNA lipoplexes (RNA(LIP)). RNA(LIP) delivers the encoded vaccine antigens to antigen-presenting cells (APCs) in lymphoid organs which results in an efficient induction of antigen-specific immune responses.

The vaccine for IV injection consists of five different RNA drug products targeting five prostate cancer associated antigens. This RNA cancer vaccine for prostate cancer consists of RBL038.1, RBL039.1, RBL040.1, RBL041.1, and RBL045.1. Each RNA cancer vaccine is composed of one RNA drug substance, which encodes the antigens Kallikrein-2 (KLK2), Kallikrein-3 (KLK3, also known as prostate specific antigen (PSA)), Acid phosphatase, prostate (ACPP, also known as prostatic acid phosphatase (PAP)), Homeobox B13 (HOXB13), and NK3 Homeobox 1 (NKX3-1), respectively, and were chosen based on their selective expression in prostate cancer. The RNAs will be reconstituted as RNA lipoplexes (RNA(LIP)) prior to administration. The RNA drug products for reconstitution may be provided in vials containing 1.1 mL of the respective RNA drug product with a concentration of 0.25 mg/mL. Sterile isotonic NaCl solution (40 mL, 0.9%) as primary diluent and liposomes (4.0 mL with a concentration of 1.4 mg/mL) as excipient for reconstitution may be delivered.

Dedicated materials such as syringes and cannulas for reconstitution as well as additional isotonic saline solution to allow for further dilution of RNA(LIP) may be sourced as clinical standard goods.

Drug Substance:

RBL038.1, beta-S-ARCA(D1)-hAg-Kozak-KLK2-GS-P2P16-GS-MITD-FI-A30L70

Encoded antigen Human Kallikrein-2 (corresponding Gene ID (HG19): uc002ptu.3, uc002ptv.3, uc002ptt.3, uc010ycl.2, uc010ycm.2, uc010yck.2, uc010eog.3)

RBL039.1, beta-S-ARCA(D1)-hAg-Kozak-KLK3-GS-P2P16-GS-MITD-FI-A30L70

Encoded antigen Human prostate specific antigen also known as human Kallikrein-3 (corresponding Gene ID (HG19: uc002 pts.1, uc002ptr.1, uc021uyi.1, uc010eof.1)

RBL040.1, beta-S-ARCA(D1)-hAg-Kozak-ACPP-GS-P2P16-GS-MITD-FI-A30L70

Encoded antigen Human prostatic acid phosphatase also known as human Acid Phosphatase Prostate (corresponding Gene ID (HG19): uc003eop.4)

RBL041.1, beta-S-ARCA(D1)-hAg-Kozak-sec-GS-HOXB13-GS-P2P16-GS-MITD-FI-A30L70

Encoded antigen Human HOMEOBOX B13 (corresponding Gene ID (HG19): uc002ioa.3r)

RBL045.1, beta-S-ARCA(D1)-hAg-Kozak-sec-GS-NKX31-GS-P2P16-GS-MITD-FI-A30L70 Encoded antigen NK3 homeobox 1 (corresponding Gene ID (HG19): uc011kzx.2)

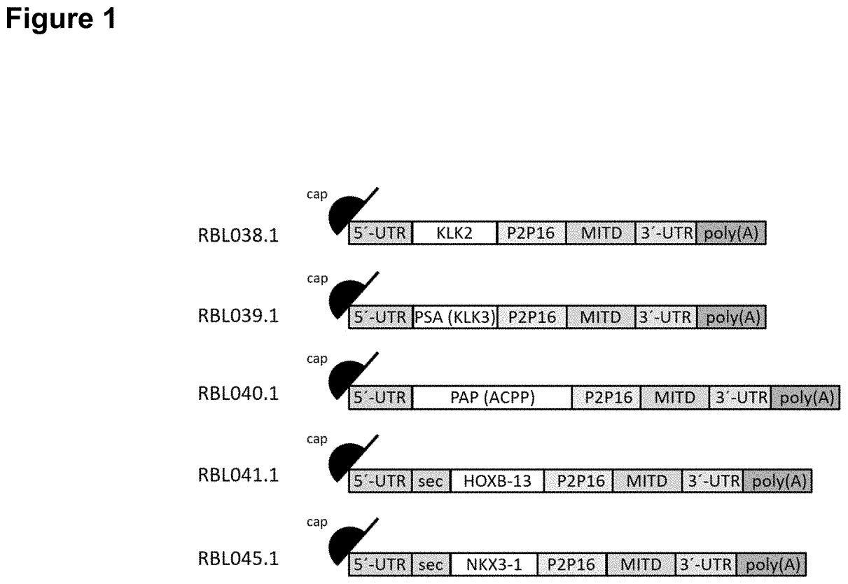

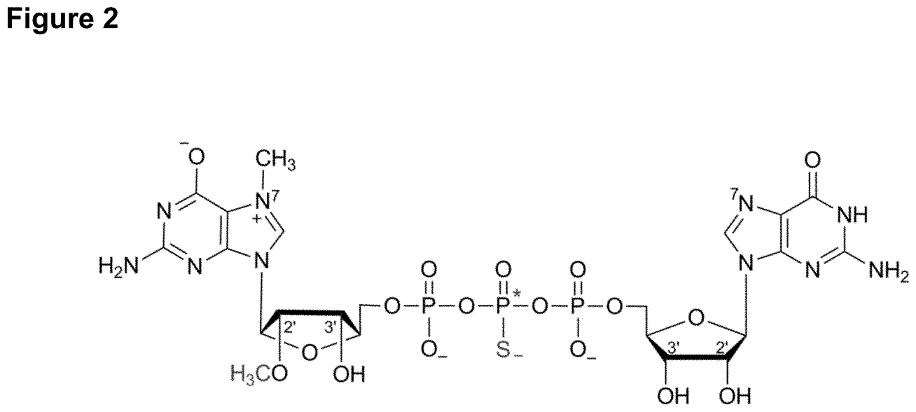

The active principle in each drug substance is a single-stranded, 5′-capped mRNA that is translated into the respective protein upon entering antigen-presenting cells (APCs). FIG. 1 schematizes the general structure of the antigen-encoding RNAs, which is determined by the respective nucleotide sequence of the linearized plasmid DNA used as template for in vitro RNA transcription. In addition to wild type or codon-optimized sequences encoding the target protein, each RNA contains common structural elements optimized for mediating maximal RNA stability and translational efficiency (5′-cap, 5′-UTR, 3′-UTR, poly(A)tail; see below). Furthermore, sec (secretory signal peptide) and MITD (MHC class I trafficking domain) are fused to the antigen-encoding regions and translated as N- or C-terminal tag, respectively. Both fusion tags were shown to improve antigen processing and presentation on both MHC class I and MHC class II complexes. For some antigens as given below, the sec fusion tag is not required and, thus, is omitted. Beta-S-ARCA(D1) (FIG. 2) is utilized as specific capping structure at the 5′-end of the RNA drug substances.

The general sequence elements of the mRNAs, as depicted in FIG. 1, are given below. KLK2, PSA (KLK3), PAP (ACPP), HOXB13, and NKX3-1: Codon-optimized sequences encoding the respective target proteins.

hAg-Kozak: 5′-UTR sequence of the human alpha-globin mRNA with an optimized ‘Kozak sequence’ to increase translational efficiency.

sec/MITD: Fusion-protein tags derived from the sequence encoding the human MHC class I complex (HLA-B51, haplotype A2, B27/B51, Cw2/Cw3), which have been shown to improve antigen processing and presentation. Sec corresponds to the 78 bp fragment coding for the secretory signal peptide, which guides translocation of the nascent polypeptide chain into the endoplasmatic reticulum. MITD corresponds to the transmembrane and cytoplasmic domain of the MHC class I molecule, also called MHC class I trafficking domain. Note that KLK2, PSA (KLK3) and PAP (ACPP) each have their own secretory signal peptide. Accordingly, no sec fusion tag has been added to these antigens.

GS/Linker: Sequences coding for short linker peptides predominantly consisting of the amino acids glycine (G) and serine (S), as commonly used for fusion proteins.

P2P16: Sequence coding for tetanus toxoid-derived helper epitopes to break immunological tolerance.

Fl element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.

A30L70: a poly(A)-tail measuring 110 nucleotides in length, consisting of a stretch of 30 adenosine residues, followed by a 10 nucleotide linker sequence and another 70 adenosine residues designed to enhance RNA stability and translational efficiency in dendritic cells. The complete nucleotide sequences of the five RNA drug substances RBL038.1, RBL039.1, RBL040.1, RBL041.1, and RBL045.1 are given below:

```
Nucleotide sequence of RBL038.1.
Nucleotide sequence is shown with individual sequence elements as indicated in bold letters.
In addition, the sequence of the translated protein is shown in italic letters below the coding
nucleotide sequence (* = stop codon).
                10 20 30 40 50 52
GGGCGAACUA GUAUUCUUCU GGUCCCCACA GACUCAGAGA GAACCCGCCA CC
                                                          hAg-Kozak
                62 72 82 92 102 112
AUGUGGGACC UGGUUCUCUC CAUCGCCUUG UCUGUGGGGU GCACUGGUGC CGUGCCCCUC
    M W D L V L S I A L S V G C T G A V P L
                                                              KLK2
              122 132 142 152 162 172
AUCCAGUCUC GGAUUGUGGG AGGCUGGGAG UGUGAGAAGC AUUCCCAACC CUGGCAGGUG
    I Q S R I V G G W E C E K H S Q P W Q V
                                                              KLK2
              182 192 202 212 222 232
GCUGUGUACA GUCAUGGAUG GGCACACUGU GGGGGUGUCC UGGUGCACCC CCAGUGGGUG
    A V Y S H G W A H C G G V L V H P Q W V
                                                              KLK2
              242 252 262 272 282 292
CUCACAGCUG CCCAUUGCCU AAAGAAGAAU AGCCAGGUCU GGCUGGGUCG GCACAACCUG
    L T A A H C L K K N S Q V W L G R H N L
                                                              KLK2
              302 312 322 332 342 352
UUUGAGCCUG AAGACACAGG CCAGAGGGUC CCUGUCAGCC ACAGCUUCCC ACACCCGCUC
    F E P E D T G Q R V P V S H S F P H P L
                                                              KLK2
              362 372 382 392 402 412
UACAAUAUGA GCCUUCUGAA GCAUCAAAGC CUUAGACCAG AUGAAGACUC CAGCCAUGAC
    Y N M S L L K H Q S L R P D E D S S H D
                                                              KLK2
              422 432 442 452 462 472
CUCAUGCUGC UCCGCCUGUC AGAGCCUGCC AAGAUCACAG AUGUUGUGAA GGUCCUGGGC
    L M L L R L S E P A K I T D V V K V L G
                                                              KLK2
              482 492 502 512 522 532
CUGCCCACCC AGGAGCCAGC ACUGGGGACC ACCUGCUACG CCUCAGGCUG GGGCAGCAUC
    L P T Q E P A L G T T C Y A S G W G S I
                                                              KLK2
              542 552 562 572 582 592
GAACCAGAGG AGUUCUUGCG CCCCAGGAGU CUUCAGUGUG UGAGCCUCCA UCUCCUGUCC
    E P E E F L R P R S L Q C V S L H L L S
                                                              KLK2
              602 612 622 632 642 652
AAUGACAUGU GUGCUAGAGC UUACUCUGAG AAGGUGACAG AGUUCAUGUU GUGUGCUGGG
    N D M C A R A Y S E K V T E F M L C A G
                                                              KLK2
              662 672 682 692 702 712
CUCUGGACAG GUGGUAAAGA CACUUGUGGG GGUGAUUCUG GGGGUCCACU UGUCUGUAAU
    L W T G G K D T C G G D S G G P L V C N
                                                              KLK2
              722 732 742 752 762 772
GGUGUGCUUC AAGGUAUCAC AUCAUGGGGC CCUGAGCCAU GUGCCCUGCC UGAAAAGCCU
    G V L Q G I T S W G P E P C A L P E K P
                                                              KLK2
              782 792 802 812 822 832
GCUGUGUACA CCAAGGUGGU GCAUUACCGG AAGUGGAUCA AGGACACCAU CGCAGCCAAC
    A V Y T K V V H Y R K W I K D T I A A N
                                                              KLK2
    835
CCC
    P
              845 855 865
GGAGGAUCCG GUGGUGGCGG CAGCGGCGGC
    G G S G G G G S G G
                        GS-linker
              875 885 895 905 915 925
AAGAAGCAGU ACAUCAAGGC CAACAGCAAG UUCAUCGGCA UCACCGAGCU GAAGAAGCUG
    K K Q Y I K A N S K F I G I T E L K K L
                                                        P2-P16 epitope
              935 945 955 965 975 985
GGAGGGGGCA AACGGGGAGG CGGCAAAAAG AUGACCAACA GCGUGGACGA CGCCCUGAUC
    G G G K R G G G K K M T N S V D D A L I
                                                        P2-P16 epitope
              995 1005 1015 1025 1035 1045
AACAGCACCA AGAUCUACAG CUACUUCCCC AGCGUGAUCA GCAAAGUGAA CCAGGGCGCU
    N S T K I Y S Y F P S V I S K V N Q G A
                                                        P2-P16 epitope
            1055 1060
CAGGGCAAGA AACUG
    Q G K K L
  P2-P16 epitope
            1070 1080 1090 1100 1102
GGCUCUAGCG GAGGGGGAGG CUCUCCUGGC GGGGGAUCUA GC
    G S S G G G G S P G G G S S
                                                          GS-linker
            1112 1122 1132 1142 1152 1162
AUCGUGGGAA UUGUGGCAGG ACUGGCAGUG CUGGCCGUGG UGGUGAUCGG AGCCGUGGUG
    I V G I V A G L A V L A V V V I G A V V
                                                              MITD
            1172 1182 1192 1202 1212 1222
GCUACCGUGA UGUGCAGACG GAAGUCCAGC GGAGGCAAGG GCGGCAGCUA CAGCCAGGCC
    A T V M C R R K S S G G K G G S Y S Q A
                                                              MITD
            1232 1242 1252 1262 1272 1273
GCCAGCUCUG AUAGCGCCCA GGGCAGCGAC GUGUCACUGA CAGCCUAGUA A
    A S S D S A Q G S D V S L T A * *
                                                              MITD
            1283 1293 1303 1313 1323 1333
CUCGAGCUGG UACUGCAUGC ACGCAAUGCU AGCUGCCCCU UUCCCGUCCU GGGUACCCCG
                                                          FI element
            1343 1353 1363 1373 1383 1393
AGUCUCCCCC GACCUCGGGU CCCAGGUAUG CUCCCACCUC CACCUGCCCC ACUCACCACC
                                                          FI element
            1403 1413 1423 1433 1443 1453
UCUGCUAGUU CCAGACACCU CCCAAGCACG CAGCAAUGCA GCUCAAAACG CUUAGCCUAG
                                                          FI element
            1463 1473 1783 1493 1503 1513
CCACACCCCC ACGGGAAACA GCAGUGAUUA ACCUUUAGCA AUAAACGAAA GUUUAACUAA
                                                          FI element
            1523 1533 1543 1553 1563 1573
GCUAUACUAA CCCCAGGGUU GGUCAAUUUC GUGCCAGCCA CACCGAGACC UGGUCCAGAG
                                                          FI element
            1583 1590
UCGCUAGCCG CGUCGCU
        FI element
            1600 1610 1620 1630 1640 1650
AAAAAAAAAA AAAAAAAAAA AAAAAAAAAA GCAUAUGACU AAAAAAAAAA AAAAAAAAAA
                                                            Poly(A)
            1660 1670 1680 1690 1700
AAAAAAAAAA AAAAAAAAAA AAAAAAAAAA AAAAAAAAAA AAAAAAAAAA
                                                            Poly(A)
Nucleotide sequence of RBL039.1.
Nucleotide sequence is shown with individual sequence elements as indicated in bold letters.
In addition, the sequence of the translated protein is shown in italic letters below the coding
nucleotide sequence (* = stop codon).
                10 20 30 40 50 52
GGGCGAACUA GUAUUCUUCU GGUCCCCACA GACUCAGAGA GAACCCGCCA CC
                                                          hAg-Kozak
                62 72 82 92 102 112
AUGUGGGUCC CGGUUGUCUU CCUCACCCUG UCCGUGACGU GGAUUGGUGC UGCACCCCUC
    M W V P V V F L T L S V T W I G A A P L
                                                              KLK3
              122 132 142 152 162 172
AUCCUGUCUC GGAUUGUGGG AGGCUGGGAG UGCGAGAAGC AUUCCCAACC CUGGCAGGUG
    I L S R I V G G W E C E K H S Q P W Q V
                                                              KLK3
              182 192 202 212 222 232
CUUGUGGCCU CUCGUGGCAG GGCAGUCUGC GGCGGUGUUC UGGUGCACCC CCAGUGGGUC
    L V A S R G R A V C G G V L V H P Q W V
                                                              KLK3
              242 252 262 272 282 292
CUCACAGCUG CCCACUGCAU CAGGAACAAA AGCGUGAUCU UGCUGGGUCG GCACAGCCUG
    L T A A H C I R N K S V I L L G R H S L
                                                              KLK3
              302 312 322 332 342 352
UUUCAUCCUG AAGACACAGG CCAGGUAUUU CAGGUCAGCC ACAGCUUCCC ACACCCGCUC
    F H P E D T G Q V F Q V S H S F P H P L
                                                              KLK3
              362 372 382 392 402 412
UACGAUAUGA GCCUCCUGAA GAAUCGAUUC CUCAGGCCAG GUGAUGACUC CAGCCACGAC
    Y D M S L L K N R F L R P G D D S S H D
                                                              KLK3
              422 432 442 452 462 472
CUCAUGCUGC UCCGCCUGUC AGAGCCUGCC GAGCUCACGG AUGCUGUGAA GGUCAUGGAC
    L M L L R L S E P A E L T D A V K V M D
                                                              KLK3
              482 492 502 512 522 532
CUGCCCACCC AGGAGCCAGC ACUGGGGACC ACCUGCUACG CCUCAGGCUG GGGCAGCAUU
    L P T Q E P A L G T T C Y A S G W G S I
                                                              KLK3
              542 552 562 572 582 592
GAACCAGAGG AGUUCUUGAC CCCAAAGAAA CUUCAGUGUG UGGACCUCCA UGUUAUUUCC
    E P E E F L T P K K L Q C V D L H V I S
                                                              KLK3
              602 612 622 632 642 652
AAUGACGUGU GUGCGCAAGU UCACCCUCAG AAGGUGACCA AGUUCAUGCU GUGUGCUGGA
    N D V C A Q V H P Q K V T K F M L C A G
                                                              KLK3
              662 672 682 692 702 712
CGCUGGACAG GGGGCAAAAG CACCUGCUCG GGUGAUUCUG GGGGCCCACU UGUCUGUAAU
    R W T G G K S T C S G D S G G P L V C N
                                                              KLK3
              722 732 742 752 762 772
GGUGUGCUUC AAGGUAUCAC GUCAUGGGGC AGUGAACCAU GUGCCCUGCC CGAAAGGCCU
    G V L Q G I T S W G S E P C A L P E R P
                                                              KLK3
              782 792 802 812 822 832
UCCCUGUACA CCAAGGUGGU GCAUUACCGG AAGUGGAUCA AGGACACCAU CGUGGCCAAC
    S L Y T K V V H Y R K W I K D T I V A N
                                                              KLK3
    835
CCC
    P
              845 855 865
GGAGGAUCCG GUGGUGGCGG CAGCGGCGGC
    G G S G G G G S G G
                        GS-linker
              875 885 895 905 915 925
AAGAAGCAGU ACAUCAAGGC CAACAGCAAG UUCAUCGGCA UCACCGAGCU GAAGAAGCUG
    K K Q Y I K A N S K F I G I T E L K K L
                                                        P2-P16 epitope
              935 945 955 965 975 985
GGAGGGGGCA AACGGGGAGG CGGCAAAAAG AUGACCAACA GCGUGGACGA CGCCCUGAUC
    G G G K R G G G K K M T N S V D D A L I
                                                        P2-P16 epitope
              995 1005 1015 1025 1035 1045
AACAGCACCA AGAUCUACAG CUACUUCCCC AGCGUGAUCA GCAAAGUGAA CCAGGGCGCU
    N S T K I Y S Y F P S V I S K V N Q G A
                                                        P2-P16 epitope
            1055 1060
CAGGGCAAGA AACUG
    Q G K K L
      P2-P16 epitope
            1070 1080 1090 1100 1102
GGCUCUAGCG GAGGGGGAGG CUCUCCUGGC GGGGGAUCUA GC
    G S S G G G G S P G G G S S
                                                            GS-linker
            1112 1122 1132 1142 1152 1162
AUCGUGGGAA UUGUGGCAGG ACUGGCAGUG CUGGCCGUGG UGGUGAUCGG AGCCGUGGUG
    I V G I V A G L A V L A V V V I G A V V
                                                              MITD
            1172 1182 1192 1202 1212 1222
GCUACCGUGA UGUGCAGACG GAAGUCCAGC GGAGGCAAGG GCGGCAGCUA CAGCCAGGCC
    A T V M C R R K S S G G K G G S Y S Q A
                                                              MITD
            1232 1242 1252 1262 1272 1273
GCCAGCUCUG AUAGCGCCCA GGGCAGCGAC GUGUCACUGA CAGCCUAGUA A
    A S S D S A Q G S D V S L T A * *
                                                              MITD
            1283 1293 1303 1313 1323 1333
CUCGAGCUGG UACUGCAUGC ACGCAAUGCU AGCUGCCCCU UUCCCGUCCU GGGUACCCCG
                                                          FI element
            1343 1353 1363 1373 1383 1393
AGUCUCCCCC GACCUCGGGU CCCAGGUAUG CUCCCACCUC CACCUGCCCC ACUCACCACC
                                                          FI element
            1403 1413 1423 1433 1443 1453
UCUGCUAGUU CCAGACACCU CCCAAGCACG CAGCAAUGCA GCUCAAAACG CUUAGCCUAG
                                                          FI element
            1463 1473 1783 1493 1503 1513
CCACACCCCC ACGGGAAACA GCAGUGAUUA ACCUUUAGCA AUAAACGAAA GUUUAACUAA
                                                          FI element
            1523 1533 1543 1553 1563 1573
GCUAUACUAA CCCCAGGGUU GGUCAAUUUC GUGCCAGCCA CACCGAGACC UGGUCCAGAG
                                                          FI element
            1583 1590
UCGCUAGCCG CGUCGCU
        FI element
            1600 1610 1620 1630 1640 1650
AAAAAAAAAA AAAAAAAAAA AAAAAAAAAA GCAUAUGACU AAAAAAAAAA AAAAAAAAAA
                                                            Poly(A)
            1660 1670 1680 1690 1700
AAAAAAAAAA AAAAAAAAAA AAAAAAAAAA AAAAAAAAAA AAAAAAAAAA
                                                            Poly(A)
Nucleotide sequence of RBL040.1.
Nucleotide sequence is shown with individual sequence elements as indicated in bold letters.
In addition, the sequence of the translated protein is shown in italic letters below the coding
nucleotide sequence (* = stop codon).
                10 20 30 40 50 52
GGGCGAACUA GUAUUCUUCU GGUCCCCACA GACUCAGAGA GAACCCGCCA CC
                                                          hAg-Kozak
                62 72 82 92 102 112
AUGAGAGCUG CUCCCCUGCU GCUGGCAAGA GCUGCUUCUC UGUCUCUGGG AUUUCUGUUU
    M R A A P L L L A R A A S L S L G F L F
                                                              ACPP
              122 132 142 152 162 172
CUGCUGUUUU UCUGGCUGGA UAGAUCUGUG CUGGCAAAAG AACUGAAAUU UGUGACCCUG
    L L F F W L D R S V L A K E L K F V T L
                                                              ACPP
              182 192 202 212 222 232
GUGUUUAGAC ACGGAGACAG AUCUCCCAUU GAUACCUUUC CCACCGAUCC CAUCAAAGAA
    V F R H G D R S P I D T F P T D P I K E
                                                              ACPP
              242 252 262 272 282 292
UCUUCUUGGC CACAGGGAUU UGGACAGCUG ACCCAGCUGG GAAUGGAGCA GCACUACGAA
    S S W P Q G F G Q L T Q L G M E Q H Y E
                                                              ACPP
              302 312 322 332 342 352
CUGGGAGAAU ACAUCAGAAA AAGAUACAGA AAAUUUCUGA AUGAAUCUUA CAAACACGAA
    L G E Y I R K R Y R K F L N E S Y K H E
                                                              ACPP
              362 372 382 392 402 412
CAGGUGUACA UCAGAUCUAC CGAUGUGGAU AGAACCCUGA UGUCUGCAAU GACCAAUCUG
    Q V Y I R S T D V D R T L M S A M T N L
                                                              ACPP
              422 432 442 452 462 472
GCUGCUCUGU UUCCCCCAGA AGGAGUGAGC AUCUGGAAUC CCAUCCUGCU GUGGCAGCCC
    A A L F P P E G V S I W N P I L L W Q P
                                                              ACPP
              482 492 502 512 522 532
AUCCCUGUGC ACACCGUGCC UCUGUCUGAA GAUCAGCUGC UGUACCUGCC UUUUAGAAAU
    I P V H T V P L S E D Q L L Y L P F R N
                                                              ACPP
              542 552 562 572 582 592
UGCCCAAGAU UUCAGGAACU UGAAUCUGAA ACCCUGAAAU CUGAAGAAUU UCAGAAAAGA
    C P R F Q E L E S E T L K S E E F Q K R
                                                              ACPP
              602 612 622 632 642 652
CUGCACCCUU ACAAAGAUUU UAUCGCAACC CUGGGAAAAC UGUCUGGACU GCACGGACAG
    L H P Y K D F I A T L G K L S G L H G Q
                                                              ACPP
              662 672 682 692 702 712
GAUCUCUUUG GAAUUUGGUC AAAAGUGUAC GAUCCUCUGU ACUGUGAAUC UGUGCACAAU
    D L F G I W S K V Y D P L Y C E S V H N
                                                              ACPP
              722 732 742 752 762 772
UUUACCCUGC CCUCUUGGGC AACCGAAGAU ACCAUGACCA AACUGAGAGA ACUGUCUGAA
    F T L P S W A T E D T M T K L R E L S E
                                                              ACPP
              782 792 802 812 822 832
CUGUCUCUGC UGUCUCUGUA CGGAAUCCAC AAACAGAAAG AAAAAUCAAG ACUGCAGGGA
    L S L L S L Y G I H K Q K E K S R L Q G
                                                              ACPP
              842 852 862 872 882 892
GGAGUGCUGG UGAAUGAAAU CCUGAAUCAC AUGAAAAGAG CAACCCAGAU CCCAUCUUAC
    G V L V N E I L N H M K R A T Q I P S Y
                                                              ACPP
              902 912 922 932 942 952
AAGAAGCUGA UCAUGUACUC UGCUCACGAU ACCACAGUGU CUGGACUGCA GAUGGCUCUG
    K K L I M Y S A H D T T V S G L Q M A L
                                                              ACPP
              962 972 982 992 1002 1012
GAUGUGUACA AUGGACUGCU GCCUCCCUAC GCUUCUUGCC ACCUGACCGA ACUGUACUUU
    D V Y N G L L P P Y A S C H L T E L Y F
                                                              ACPP
            1022 1032 1042 1052 1062 1072
GAAAAAGGAG AAUACUUUGU GGAAAUGUAC UACAGAAAUG AAACCCAGCA CGAACCUUAC
    E K G E Y F V E M Y Y R N E T Q H E P Y
                                                              ACPP
            1082 1092 1102 1112 1122 1132
CCCCUGAUGC UGCCUGGAUG CUCUCCCUCU UGCCCUCUGG AAAGAUUUGC UGAACUGGUG
    P L M L P G C S P S C P L E R F A E L V
                                                              ACPP
            1142 1152 1162 1172 1182 1192
GGACCUGUGA UCCCUCAGGA UUGGUCCACA GAAUGCAUGA CCACCAAUUC UCACCAGGUG
    G P V I P Q D W S T E C M T T N S H Q V
                                                              ACPP
            1202 1212 1222 1232 1242 1252
CUGAAAGUGA UCUUUGCUGU GGCUUUUUGC CUGAUCUCUG CUGUGCUGAU GGUGCUGCUG
    L K V I F A V A F C L I S A V L M V L L
                                                              ACPP
            1262 1272 1282 1292 1302 1306
UUUAUCCACA UCAGGAGAGG ACUGUGCUGG CAGAGAGAAU CUUACGGAAA UAUC
    F I H I R R G L C W Q R E S Y G N I
                                                              ACPP
            1316 1326 1336
GGAGGAUCCG GUGGUGGCGG CAGCGGCGGC
    G G S G G G G S G G
                        GS-linker
            1346 1356 1366 1376 1386 1396
AAGAAGCAGU ACAUCAAGGC CAACAGCAAG UUCAUCGGCA UCACCGAGCU GAAGAAGCUG
    K K Q Y I K A N S K F I G I T E L K K L
                                                        P2-P16 epitope
            1406 1416 1426 1436 1446 1456
GGAGGGGGCA AACGGGGAGG CGGCAAAAAG AUGACCAACA GCGUGGACGA CGCCCUGAUC
    G G G K R G G G K K M T N S V D D A L I
                                                        P2-P16 epitope
            1466 1476 1486 1496 1506 1516
AACAGCACCA AGAUCUACAG CUACUUCCCC AGCGUGAUCA GCAAAGUGAA CCAGGGCGCU
    N S T K I Y S Y F P S V I S K V N Q G A
                                                        P2-P16 epitope
            1526 1531
CAGGGCAAGA AACUG
    Q G K K L
      P2-P16 epitope
            1541 1551 1561 1571 1573
GGCUCUAGCG GAGGGGGAGG CUCUCCUGGC GGGGGAUCUA GC
    G S S G G G G S P G G G S S
                                                            GS-linker
            1583 1593 1603 1613 1623 1633
AUCGUGGGAA UUGUGGCAGG ACUGGCAGUG CUGGCCGUGG UGGUGAUCGG AGCCGUGGUG
    I V G I V A G L A V L A V V V I G A V V
                                                              MITD
            1643 1653 1663 1673 1683 1693
GCUACCGUGA UGUGCAGACG GAAGUCCAGC GGAGGCAAGG GCGGCAGCUA CAGCCAGGCC
    A T V M C R R K S S G G K G G S Y S Q A
                                                              MITD
            1703 1713 1723 1733 1743 1744
GCCAGCUCUG AUAGCGCCCA GGGCAGCGAC GUGUCACUGA CAGCCUAGUA A
    A S S D S A Q G S D V S L T A * *
                                                              MITD
            1754 1764 1774 1784 1794 1804
CUCGAGCUGG UACUGCAUGC ACGCAAUGCU AGCUGCCCCU UUCCCGUCCU GGGUACCCCG
                                                          FI element
            1814 1824 1834 1844 1854 1864
AGUCUCCCCC GACCUCGGGU CCCAGGUAUG CUCCCACCUC CACCUGCCCC ACUCACCACC
                                                          FI element
            1874 1884 1894 1904 1914 1924
UCUGCUAGUU CCAGACACCU CCCAAGCACG CAGCAAUGCA GCUCAAAACG CUUAGCCUAG
                                                          FI element
            1934 1944 1954 1964 1974 1984
CCACACCCCC ACGGGAAACA GCAGUGAUUA ACCUUUAGCA AUAAACGAAA GUUUAACUAA
                                                          FI element
            1994 2004 2014 2024 2034 2044
GCUAUACUAA CCCCAGGGUU GGUCAAUUUC GUGCCAGCCA CACCGAGACC UGGUCCAGAG
                                                          FI element
            2054 2061
UCGCUAGCCG CGUCGCU
          FI element
            2071 2081 2091 2101 2111 2121
AAAAAAAAAA AAAAAAAAAA AAAAAAAAAA GCAUAUGACU AAAAAAAAAA AAAAAAAAAA
                                                            Poly(A)
            2131 2141 2151 2161 2171
AAAAAAAAAA AAAAAAAAAA AAAAAAAAAA AAAAAAAAAA AAAAAAAAAA
                                                            Poly(A)
Nucleotide sequence of RBL041.1.
Nucleotide sequence is shown with individual sequence elements as indicated in bold letters.
In addition, the sequence of the translated protein is shown in italic letters below the coding
nucleotide sequence (* = stop codon).
                10 20 30 40 50 52
GGGCGAACUA GUAUUCUUCU GGUCCCCACA GACUCAGAGA GAACCCGCCA CC
                                                          hAg-Kozak
                62 72 82 92 102 112
AUGAGAGUGA UGGCCCCCAG AACCCUGAUC CUGCUGCUGU CUGGCGCCCU GGCCCUGACA
    M R V M A P R T L I L L L S G A L A L T
                                                                Sec
              122 130
GAGACAUGGG CCGGAAGC
    E T W A G S
                Sec
              140 150 160
GGCGGCUCUG GAGGAGGCGG CUCCGGAGGC
    G G S G G G G S G G
                        GS-linker
              170 180 190 200 210 220
AUGGAGCCCG GCAAUUAUGC CACCUUGGAU GGAGCCAAGG AUAUCGAAGG CUUGCUGGGA
    M E P G N Y A T L D G A K D I E G L L G
                                                            HOXB13
              230 240 250 260 270 280
GCGGGAGGGG GGCGGAAUCU GGUCGCCCAC UCCCCUCUGA CCAGCCACCC AGCGGCGCCU
    A G G G R N L V A H S P L T S H P A A P
                                                            HOXB13
              290 300 310 320 330 340
ACGCUGAUGC CUGCUGUCAA CUAUGCCCCC UUGGAUCUGC CAGGCUCGGC GGAGCCGCCA
    T L M P A V N Y A P L D L P G S A E P P
                                                            HOXB13
              350 360 370 380 390 400
AAGCAAUGCC ACCCAUGCCC UGGGGUGCCC CAGGGGACGU CCCCAGCUCC CGUGCCUUAU
    K Q C H P C P G V P Q G T S P A P V P Y
                                                            HOXB13
              410 420 430 440 450 460
GGUUACUUUG GAGGCGGGUA CUACUCCUGC CGAGUGUCCC GGAGUUCGCU GAAACCCUGU
    G Y F G G G Y Y S C R V S R S S L K P C
                                                            HOXB13
              470 480 490 500 510 520
GCCCAGGCAG CCACCCUGGC CGCGUACCCC GCGGAGACUC CCACGGCCGG GGAGGAGUAC
    A Q A A T L A A Y P A E T P T A G E E Y
                                                            HOXB13
              530 540 550 560 570 580
CCCAGCCGCC CCACUGAGUU UGCCUUCUAU CCGGGAUAUC CGGGAACCUA CCAGCCUAUG
    P S R P T E F A F Y P G Y P G T Y Q P M
                                                            HOXB13
              590 600 610 620 630 640
GCCAGUUACC UGGACGUGUC UGUGGUGCAG ACUCUGGGUG CUCCUGGAGA ACCGCGACAU
    A S Y L D V S V V Q T L G A P G E P R H
                                                            HOXB13
              650 660 670 680 690 700
GACUCCCUGU UGCCUGUGGA CAGUUACCAG UCUUGGGCUC UCGCUGGUGG CUGGAACAGC
    D S L L P V D S Y Q S W A L A G G W N S
                                                            HOXB13
              710 720 730 740 750 760
CAGAUGUGUU GCCAGGGAGA ACAGAACCCA CCAGGUCCCU UUUGGAAGGC AGCAUUUGCA
    Q M C C Q G E Q N P P G P F W K A A F A
                                                            HOXB13
              770 780 790 800 810 820
GACUCCAGCG GGCAGCACCC UCCUGACGCC UGCGCCUUUC GUCGCGGCCG CAAGAAACGC
    D S S G Q H P P D A C A F R R G R K K R
                                                            HOXB13
              830 840 850 860 870 880
AUUCCGUACA GCAAGGGGCA GUUGCGGGAG CUGGAGCGGG AGUAUGCGGC UAACAAGUUC
    I P Y S K G Q L R E L E R E Y A A N K F
                                                            HOXB13
              890 900 910 920 930 940
AUCACCAAGG ACAAGAGGCG CAAGAUCUCG GCAGCCACCA GCCUCUCGGA GCGCCAGAUU
    I T K D K R R K I S A A T S L S E R Q I
                                                            HOXB13
              950 960 970 980 990 1000
ACCAUCUGGU UUCAGAACCG CCGGGUCAAA GAGAAGAAGG UUCUCGCCAA GGUGAAGAAC
    T I W F Q N R R V K E K K V I A K V K N
                                                            HOXB13
            1010 1012
AGCGCUACCC CU
    S A T P
        HOXB13
            1022 1032 1042
GGAGGAUCCG GUGGUGGCGG CAGCGGCGGC
    G G S G G G G S G G
                        GS-linker
            1052 1062 1072 1082 1092 1102
AAGAAGCAGU ACAUCAAGGC CAACAGCAAG UUCAUCGGCA UCACCGAGCU GAAGAAGCUG
    K K Q Y I K A N S K F I G I T E L K K L
                                                        P2-P16 epitope
            1112 1122 1132 1142 1152
GGAGGGGGCA AACGGGGAGG CGGCAAAAAG AUGACCAACA GCGUGGACGA CGCCCUGAUC
    G G G K R G G G K K M T N S V D D A L I
                                                        P2-P16 epitope
            1172 1182 1192 1202 1212 1222
AACAGCACCA AGAUCUACAG CUACUUCCCC AGCGUGAUCA GCAAAGUGAA CCAGGGCGCU
    N S T K I Y S Y F P S V I S K V N Q G A
                                                        P2-P16 epitope
            1232 1237
CAGGGCAAGA AACUG
    Q G K K L
      P2-P16 epitope
            1247 1257 1267 1277 1279
GGCUCUAGCG GAGGGGGAGG CUCUCCUGGC GGGGGAUCUA GC
    G S S G G G G S P G G G S S
                                                          GS-linker
            1289 1299 1309 1319 1329 1339
AUCGUGGGAA UUGUGGCAGG ACUGGCAGUG CUGGCCGUGG UGGUGAUCGG AGCCGUGGUG
    I V G I V A G L A V L A V V V I G A V V
                                                              MITD
            1349 1359 1369 1379 1389 1399
GCUACCGUGA UGUGCAGACG GAAGUCCAGC GGAGGCAAGG GCGGCAGCUA CAGCCAGGCC
    A T V M C R R K S S G G K G G S Y S Q A
                                                              MITD
            1409 1419 1429 1439 1449 1450
GCCAGCUCUG AUAGCGCCCA GGGCAGCGAC GUGUCACUGA CAGCCUAGUA A
    A S S D S A Q G S D V S L T A * *
                                                              MITD
            1460 1470 1480 1490 1500 1510
CUCGAGCUGG UACUGCAUGC ACGCAAUGCU AGCUGCCCCU UUCCCGUCCU GGGUACCCCG
                                                          FI element
            1520 1530 1540 1550 1560 1570
AGUCUCCCCC GACCUCGGGU CCCAGGUAUG CUCCCACCUC CACCUGCCCC ACUCACCACC
                                                          FI element
            1580 1590 1600 1610 1620 1630
UCUGCUAGUU CCAGACACCU CCCAAGCACG CAGCAAUGCA GCUCAAAACG CUUAGCCUAG
                                                          FI element
            1640 1650 1660 1670 1680 1690
CCACACCCCC ACGGGAAACA GCAGUGAUUA ACCUUUAGCA AUAAACGAAA GUUUAACUAA
                                                          FI element
            1700 1710 1720 1730 1740 1750
GCUAUACUAA CCCCAGGGUU GGUCAAUUUC GUGCCAGCCA CACCGAGACC UGGUCCAGAG
                                                          FI element
            1760 1767
UCGCUAGCCG CGUCGCU
  FI element
            1777 1787 1797 1807 1817 1827
AAAAAAAAAA AAAAAAAAAA AAAAAAAAAA GCAUAUGACU AAAAAAAAAA AAAAAAAAAA
                                                            Poly(A)
            1837 1847 1857 1867 1877
AAAAAAAAAA AAAAAAAAAA AAAAAAAAAA AAAAAAAAAA AAAAAAAAAA
                                                            Poly(A)
Nucleotide sequence of RBL045.1.
Nucleotide sequence is shown with individual sequence elements as indicated in bold letters.
In addition, the sequence of the translated protein is shown in italic letters below the coding
nucleotide sequence (* = stop codon).
                10 20 30 40 50 52
GGGCGAACUA GUAUUCUUCU GGUCCCCACA GACUCAGAGA GAACCCGCCA CC
                                                          hAg-Kozak
                62 72 82 92 102 112
AUGAGAGUGA UGGCCCCCAG AACCCUGAUC CUGCUGCUGU CUGGCGCCCU GGCCCUGACA
    M R V M A P R T L I L L L S G A L A L T
                                                                Sec
              122 130
GAGACAUGGG CCGGAAGC
    E T W A G S
                Sec
              140 150 160
GGCGGCUCUG GAGGAGGCGG CUCCGGAGGC
    G G S G G G G S G G
                        GS-linker
              170 180 190 200 210 220
AUGCUGAGAG UGCCCGAACC UAGACCUGGC GAGGCUAAAG CUGAAGGCGC UGCUCCUCCU
    M L R V P E P R P G E A K A E G A A P P
                                                            NKX3-1
              230 240 250 260 270 280
ACACCUAGCA AGCCUCUGAC CAGCUUCCUG AUCCAAGACA UCCUGAGAGA UGGCGCCCAG
    T P S K P L T S F L I Q D I L R D G A Q
                                                            NKX3-1
              290 300 310 320 330 340
AGACAAGGCG GCAGAACAAG CUCUCAGAGA CAGAGAGAUC CCGAGCCUGA GCCUGAACCA
    R Q G G R T S S Q R Q R D P E P E P E P
                                                            NKX3-1
              350 360 370 380 390 400
GAACCUGAAG GCGGAAGAUC UAGAGCCGGC GCUCAGAACG AUCAGCUGAG CACAGGACCU
    E P E G G R S R A G A Q N D Q L S T G P
                                                            NKX3-1
              410 420 430 440 450 460
AGAGCCGCUC CUGAGGAAGC CGAAACACUG GCCGAAACCG AGCCAGAGAG ACACCUGGGC
    R A A P E E A E T L A E T E P E R H L G
                                                            NKX3-1
              470 480 490 500 510 520
AGCUACCUGC UGGACAGCGA GAAUACUUCU GGCGCCCUGC CUAGACUGCC CCAGACACCU
    S Y L L D S E N T S G A L P R L P Q T P
                                                            NKX3-1
              530 540 550 560 570 580
AAACAGCCUC AGAAGCGGAG CAGAGCCGCC UUUAGCCACA CACAAGUGAU CGAGCUGGAA
    K Q P Q K R S R A A E S H T Q V I E L E
                                                            NKX3-1
              590 600 610 620 630 640
CGGAAGUUCA GCCACCAGAA GUACCUGAGC GCCCCUGAAA GAGCCCACCU GGCCAAGAAU
    R K F S H Q K Y L S A P E R A H L A K N
                                                            NKX3-1
              650 660 670 680 690 700
CUGAAGCUGA CCGAGACUCA AGUGAAGAUC UGGUUCCAGA ACCGGCGGUA CAAGACCAAG
    L K L T E T Q V K I W F Q N R R Y K T K
                                                            NKX3-1
              710 720 730 740 750 760
CGGAAGCAGC UGUCAAGCGA GCUGGGCGAC CUGGAAAAGC ACAGCUCUCU GCCCGCUCUG
    R K Q L S S E L G D L E K H S S L P A L
                                                            NKX3-1
              770 780 790 800 810 820
AAAGAGGAAG CCUUCUCCAG AGCCAGCCUG GUGUCCGUGU ACAACAGCUA CCCUUACUAC
    K E E A E S R A S L V S V Y N S Y P Y Y
                                                            NKX3-1
              830 840 850 860 862
CCCUACCUGU ACUGCGUCGG CUCUUGGAGC CCUGCCUUUU GG
    P Y L Y C V G S W S P A F W
                                                            NKX3-1
              872 882 892
GGAGGAUCCG GUGGUGGCGG CAGCGGCGGC
    G G S G G G G S G G
                        GS-linker
              902 912 922 932 942 952
AAGAAGCAGU ACAUCAAGGC CAACAGCAAG UUCAUCGGCA UCACCGAGCU GAAGAAGCUG
    K K Q Y I K A N S K F I G I T E L K K L
                                                        P2-P16 epitope
              962 972 982 992 1002 1012
GGAGGGGGCA AACGGGGAGG CGGCAAAAAG AUGACCAACA GCGUGGACGA CGCCCUGAUC
    G G G K R G G G K K M T N S V D D A L I
                                                        P2-P16 epitope
            1022 1032 1042 1052 1062 1072
AACAGCACCA AGAUCUACAG CUACUUCCCC AGCGUGAUCA GCAAAGUGAA CCAGGGCGCU
    N S T K I Y S Y F P S V I S K V N Q G A
                                                        P2-P16 epitope
            1082 1087
CAGGGCAAGA AACUG
    Q G K K L
      P2-P16 epitope
            1097 1107 1117 1127 1129
GGCUCUAGCG GAGGGGGAGG CUCUCCUGGC GGGGGAUCUA GC
    G S S G G G G S P G G G S S
                                                          GS-linker
            1139 1149 1159 1169 1179 1189
AUCGUGGGAA UUGUGGCAGG ACUGGCAGUG CUGGCCGUGG UGGUGAUCGG AGCCGUGGUG
    I V G I V A G L A V L A V V V I G A V V
                                                              MITD
            1199 1209 1219 1229 1239 1249
GCUACCGUGA UGUGCAGACG GAAGUCCAGC GGAGGCAAGG GCGGCAGCUA CAGCCAGGCC
    A T V M C R R K S S G G K G G S Y S Q A
                                                              MITD
            1259 1269 1279 1289 1299 1300
GCCAGCUCUG AUAGCGCCCA GGGCAGCGAC GUGUCACUGA CAGCCUAGUA A
    A S S D S A Q G S D V S L T A * *
                                                              MITD
            1310 1320 1330 1340 1350 1360
CUCGAGCUGG UACUGCAUGC ACGCAAUGCU AGCUGCCCCU UUCCCGUCCU GGGUACCCCG
                                                          FI element
            1370 1380 1390 1400 1410 1420
AGUCUCCCCC GACCUCGGGU CCCAGGUAUG CUCCCACCUC CACCUGCCCC ACUCACCACC
                                                          FI element
            1430 1440 1450 1460 1470 1480
UCUGCUAGUU CCAGACACCU CCCAAGCACG CAGCAAUGCA GCUCAAAACG CUUAGCCUAG
                                                          FI element
            1490 1500 1510 1520 1530 1540
CCACACCCCC ACGGGAAACA GCAGUGAUUA ACCUUUAGCA AUAAACGAAA GUUUAACUAA
                                                          FI element
            1550 1560 1570 1580 1590 1600
GCUAUACUAA CCCCAGGGUU GGUCAAUUUC GUGCCAGCCA CACCGAGACC UGGUCCAGAG
                                                          FI element
            1610 1617
UCGCUAGCCG CGUCGCU
        FI element
            1627 1637 1647 1657 1667 1677
AAAAAAAAAA AAAAAAAAAA AAAAAAAAAA GCAUAUGACU AAAAAAAAAA AAAAAAAAAA
                                                            Poly(A)
            1687 1697 1707 1717 1727
AAAAAAAAAA AAAAAAAAAA AAAAAAAAAA AAAAAAAAAA AAAAAAAAAA
                                                            Poly(A)
```

The individual plasmid DNAs for the production of RBL038.1(pST4-hAg-Kozak-KLK2-GS-P2P16-GS-MITD-FI-A30L70), RBL039.1 (pST4-hAg-Kozak-KLK3-GS-P2P16-GS-MITD-FI-A30L70), RBL040.1 (pST4-hAg-Kozak-ACPP-GS-P2P16-GS-MITD-FI-A30L70), RBL041.1 (pST4-hAg-Kozak-sec-GS-HOXB13-GS-P2P16-GS-MITD-FI-A30L70), and RBL045.1 (pST4-hAg-Kozak-sec-GS-NKX3-1-GS-P2P16-GS-MITD-FI-A30L70) were generated using a combination of gene synthesis and recombinant DNA technology. In addition to the sequence coding for the transcribed regions, the plasmid DNAs contain a promoter for the T7 RNA polymerase, the recognition sequence for the class IIs endonuclease used for linearization, the Kanamycin resistance gene, and an origin of replication (ori).

The plasmid DNA pST4-hAg-Kozak-sec-GS-SIINFEKL-GS-Ova-GS-P2P16-GS-MITD-FI-A30L70 served as starting point for the generation of the DNA templates for RBL038.1, RBL039.1, RBL040.1, RBL041.1, and RBL045.1.

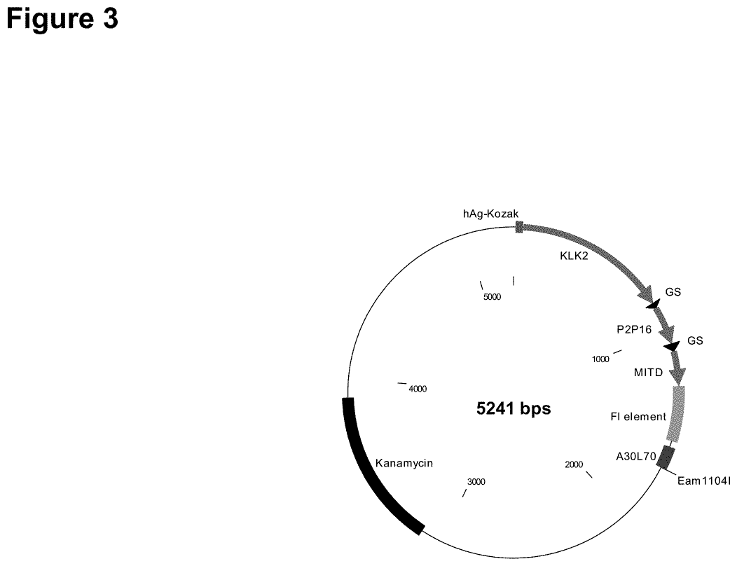

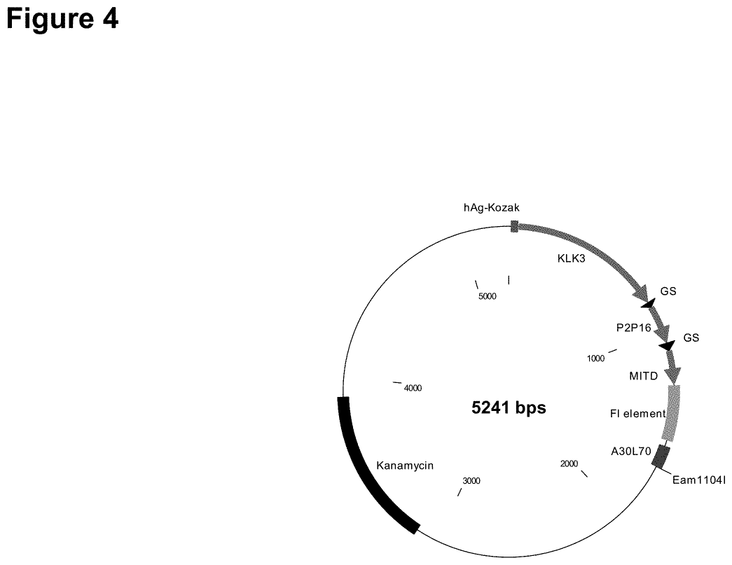

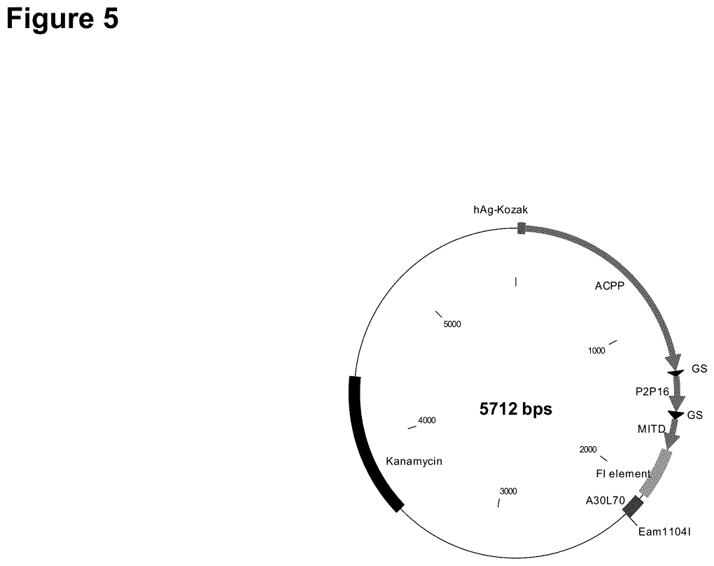

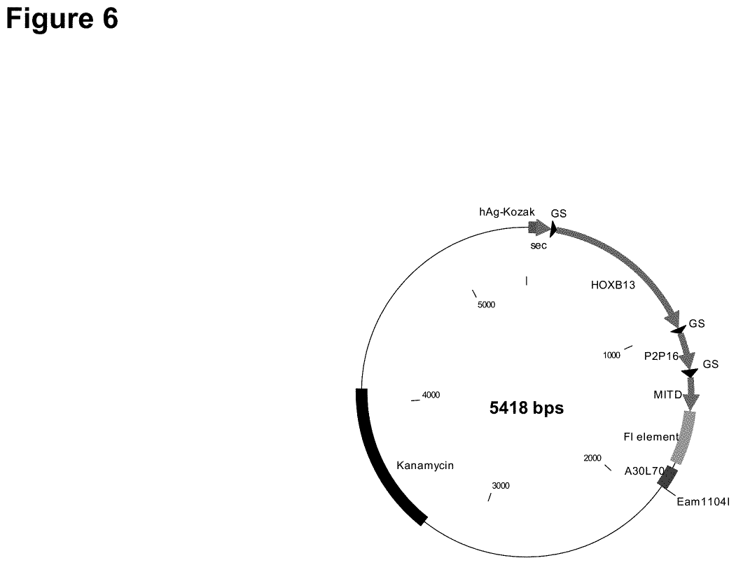

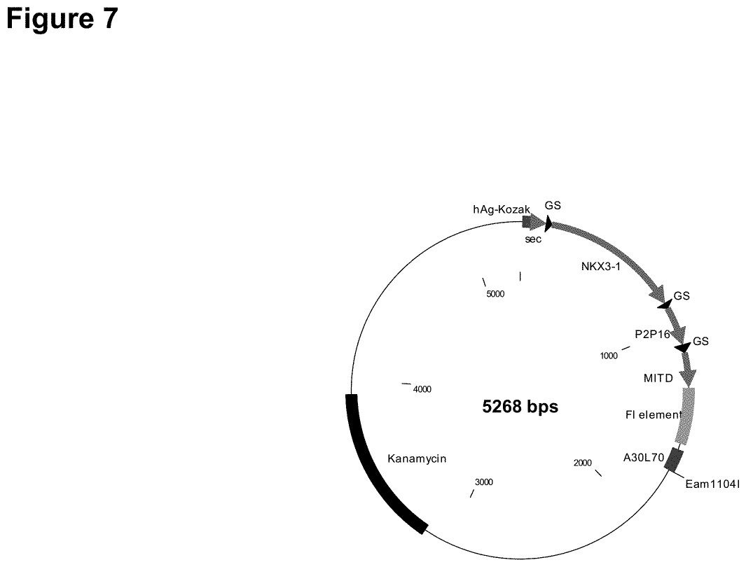

Vector maps are shown in FIGS. 3 to 7.

The circular plasmid DNA is linearized with a suitable restriction enzyme in order to obtain the starting material for RNA transcription. Here, the enzyme Eam1104I (Thermo Fisher Scientific Baltics UAB, Vilnius, Lithuania) was selected, because linearization with such a class IIs restriction endonuclease allows transcription of RNAs encoding a‘free’ poly(A)-tail, i.e., having no additional nucleotides at the 3′-end. It could be demonstrated that this gives higher protein expression.

The RNA(LIP) product may be prepared in a two-step procedure comprising (i) dilution of RNA concentrate with NaCl solution, and (ii) RNA lipoplex formation by addition of liposomes. For RNA(LIP) preparation, liposomes may be added to the diluted RNA. As lipids, the synthetic cationic lipid DOTMA and the naturally occurring phospholipid DOPE may be employed. The product for IV injection is a formulation with pharmaceutical and physiological characteristics that allow selective targeting of RNA to APCs mainly residing in the spleen. The RNA lipoplexes are formed by first condensing the RNA with a suitable ionic environment and subsequent incubation with positively charged liposomes.

For RNA condensing, various monovalent and divalent ions, peptides, and buffers were applied in various concentrations. Monovalent ions like sodium and ammonium were tested in concentrations up to 1.5 M. Divalent ions, in particular Ca²⁺, Mg²⁺, Zn²⁺, and Fe²⁺ were tested in concentrations up to 50 mM. Furthermore, various commercially available buffer solutions were tested.

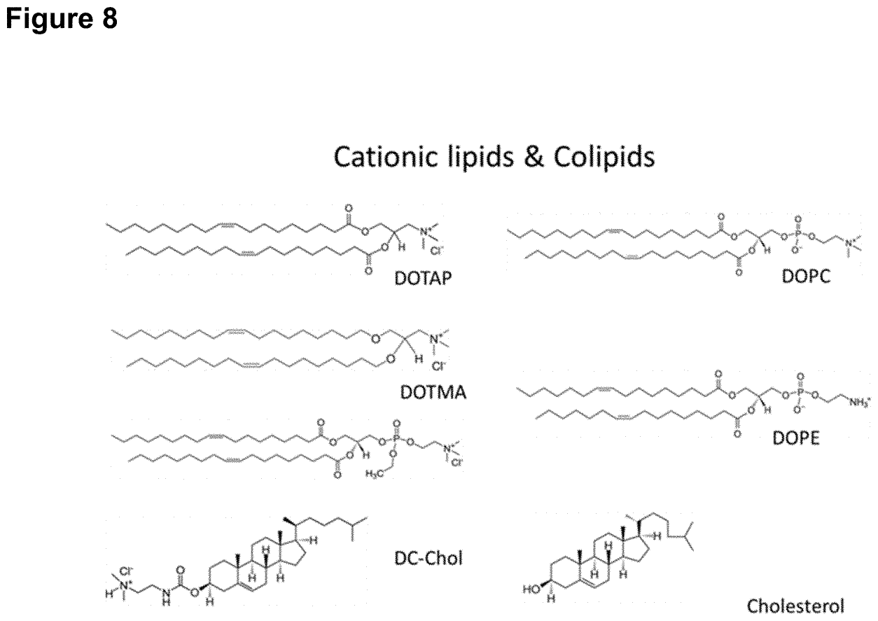

For RNA(LIP) formation, liposomes comprising a cationic lipid and different co-lipids were extensively tested. Liposomes which differ in charge, phase state, size, lamellarity, and surface functionalization were investigated. Only lipid components that are available in GMP grade, and which have previously been tested in clinical trials or which are used for approved products on the market were considered (FIG. 8).

Using the above described liposome components, RNA lipoplexes were assembled with different cationic lipid:RNA and different charge ratios, where the charge ratio was calculated from the number of positive charges from the lipids and the negative charges from the RNA nucleotides, i.e., from the RNA phosphate groups. More specifically, the calculation of the charge ratio was performed as follows:

RNA was assumed to consist of nucleotides with a mean molar mass of 330 Da, each carrying a phosphate group with one negative charge. Therefore, a solution of 1 mg/mL of RNA accounts for approximately 3 mM in negative charges. On the other hand, one positive charge per monovalent cationic lipid was taken into account. For example, the cationic lipid DOTMA has a molar mass of 670 Da, liposomes with a DOTMA concentration of 2 mg/mL were attributed a concentration of positive charges of 3 mM. Therefore, in this case the (+: −) charge ratio was taken as 1:1. The concentration of the uncharged co-lipids, which in most cases were present, does not contribute to this calculation.

Chemical and physicochemical properties of the liposomes and the RNA lipoplexes formed on this basis (i.e., regarding chemical composition, particle size, zeta potential) were thoroughly investigated. For regular control of the product quality, chemical composition was determined by HPLC analysis and the particle size was measured by photon correlation spectroscopy (PCS).

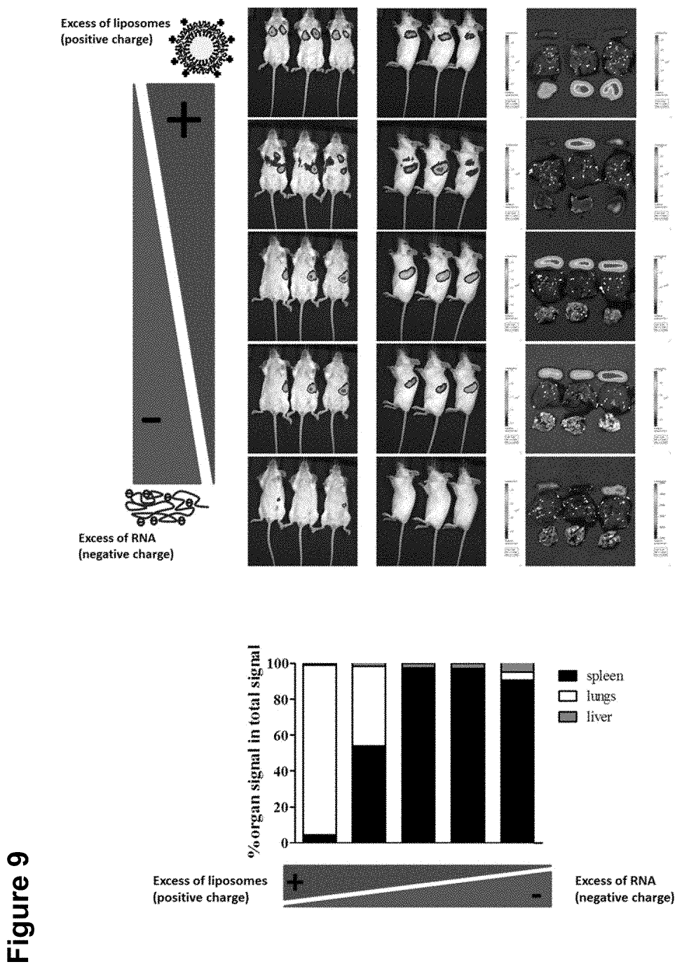

Also the zeta potential was measured by PCS. Furthermore, electron microscopy, small angle X-ray scattering (SAXS), calorimetry, field-flow fractionation, analytical ultracentrifugation, and spectroscopic techniques were applied in the course of formulation development. By this procedure, optimized formulations for further pharmaceutical development were identified. Suitable liposome formulations were tested in vitro and in vivo. In order to optimize targeting to APCs mostly residing in the spleen, expression of luciferase as a reporter gene was observed in vivo. It could be shown, that colloidal stable nanoparticulate lipoplex formulations with discrete particle sizes could be formed at suitable charge ratios (excess of negative or positive charge). Furthermore, it has been shown in vivo, that negatively charged luciferase-RNA lipoplex formulations displayed high selectivity for the spleen, which serves as a reservoir for professional APCs. By changing the charge ratio, the selectivity of luciferase expression in the spleen could be adjusted as desired, as shown in FIG. 9, where the organ selectivity of RNA lipoplexes from the same liposomes with different mixing ratios of cationic lipid to RNA is displayed. The observation that negatively charged lipoplexes target splenic APCs could be verified for a large number of lipid compositions. Liposomes consisting of the cationic lipid DOTMA and the helper phospholipid DOPE were identified to be most appropriate in terms of particle characteristics for formation of suitable RNA lipoplexes for the intended splenic APC targeting. Optimized selectivity and efficacy of spleen targeting is observed at a slight excess of negative charge constituted by an excess of RNA. RNA lipoplexes which were slightly more positively charged and displayed comparable efficacy were not suitable for development of a pharmaceutical product as they were colloidally too instable and there was a high risk of aggregation and precipitation under these conditions.

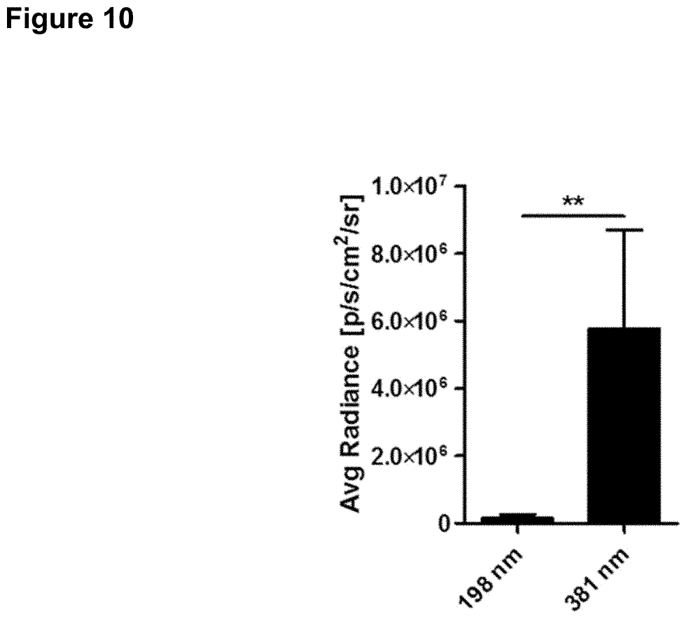

Furthermore, it could be shown that for a given RNA the biological activity of the formulations increased with the particle size of the RNA lipoplexes. More specifically, it could be shown, that RNA lipoplexes formed from larger liposomes (e.g., approx. 400 nm) were itself larger than those prepared with smaller liposomes (e.g., approx. 200 nm) and displayed a higher biological activity (FIG. 10). Therefore, liposomes larger than 200 nm are preferably used for RNA(LIP) formation.

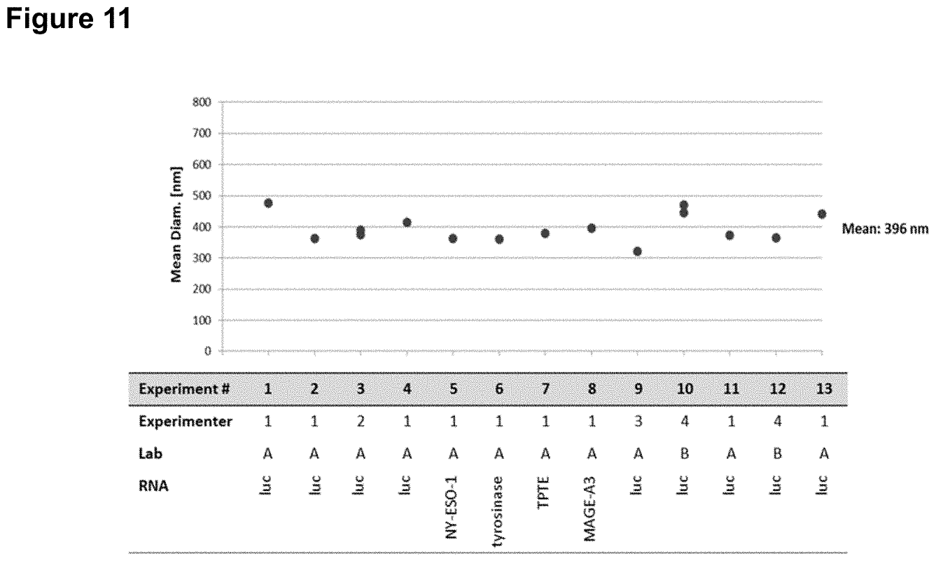

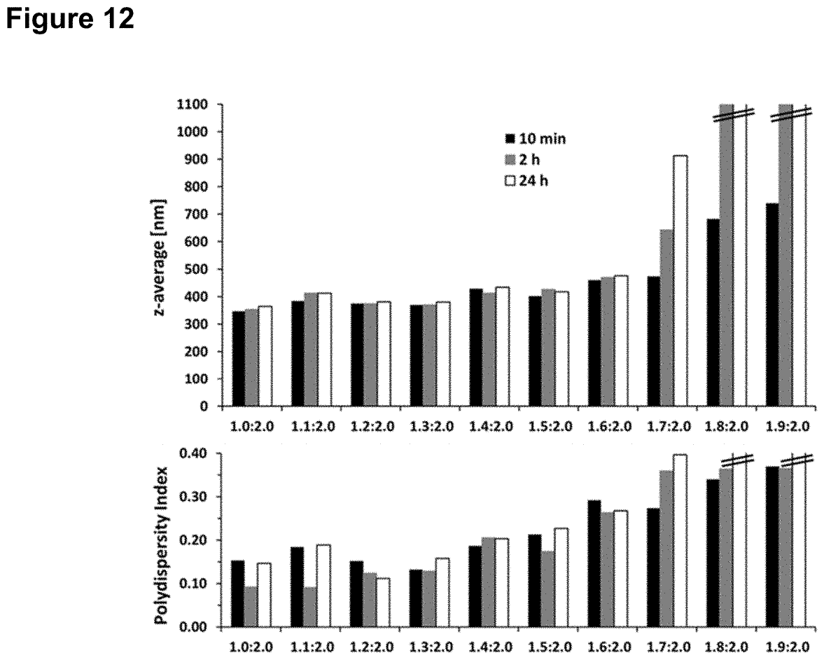

On the basis of the findings described above, a robust and reproducible protocol for RNA(LIP) preparation has been developed. By using the components as specified and the defined preparation protocol, RNA lipoplexes form by self-assembly to the intended physicochemical characteristics and biological activity. As an example, particle sizes of RNA lipoplexes from various independent preparations are given in FIG. 11. Limited spread of obtained RNA lipoplex particle sizes demonstrates the robustness of the reconstitution procedure. In order to determine the limits and the robustness of RNA(LIP) preparation, particle sizes were measured for different charge ratios from 1.0:2.0 to 1.9:2.0 (mixing ratios between cationic lipid and nucleotides). In FIG. 12, results from size measurements of RNA lipoplexes after mixing of liposomes with RNA at various ratios are shown. Particle size was measured at different time points after RNA(LIP) preparation. For ratios from 1.0:2.0 to 1.6:2.0, comparable particle sizes which are stable over time are obtained. For ratios of 1.7:2.0 and higher, the particle size of the RNA lipoplexes increases, both initially and over time. This finding is most pronounced after 24 h.

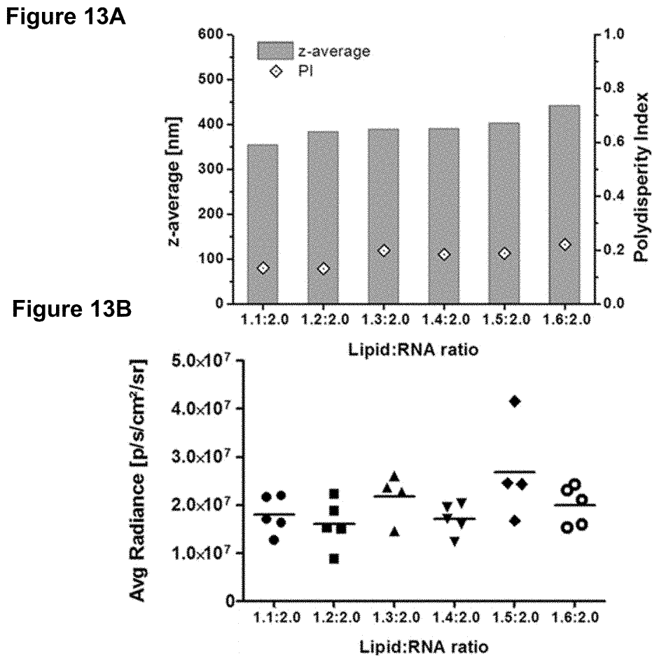

On the basis of these data, the charge ratios between 1.0:2.0 and 1.6:2.0 were considered suitable to obtain acceptable particle characteristics for the RNA lipoplex products. At higher ratios (1.7:2.0 and above), the particle size increased, leading to potentially deviating product quality. Towards lower charge ratios no change in particle characteristics was observed, however, lower ratios were not considered because of potentially lower activity in that range (data not shown). The experiment has been repeated for the range of 1.1:2.0 to 1.6:2.0 and, in addition to the size measurements (FIG. 13A), the biological activity was investigated (FIG. 13B). In line with previous experiments, particle sizes were virtually constant. The same holds true for the biological activity (luciferase expression). To summarize, RNA lipoplexes of all tested charge ratios have delivered RNA to APCs without significant changes in physicochemical properties or biological performance. Therefore, the range between 1.1:2.0 and 1.6:2.0 is considered to result in RNA lipoplexes of equivalent quality.

### Example 2: Nonclinical Data

This example reviews the nonclinical studies that were conducted to elucidate the mode of action, pharmacodynamics, anti-tumor activity, pharmacokinetics and potential toxicity of the RNA(LIP) vaccine. The key findings are summarised in Table 1.

The first part of this section provides a brief overview of the scientific foundation and preparatory work for the development of the vaccine platform itself (Section 1) and a brief overview of target characteristics of W_pro1 targets.

The following section describes the studies on the primary pharmacodynamics of RNA(LIP), namely the induction of antigen-specific T cells in vivo, and the anti-tumor activity of RNA(LIP) vaccination (Section 2).

The studies on secondary pharmacodynamics lay out the results of testing for RNA(LIP)-mediated induction of pro-inflammatory cytokines (Section 3). A pharmacodynamics non-GLP study in cynomolgus monkeys was conducted to refine cytokine kinetics data, as well as hematological changes that have been observed in mice. In vitro studies analyzing the cytokine secretion of human and cynomolgus blood cells after incubation with RNA(LIP) preparations are also summarised in this section.

Safety pharmacology studies for respiratory and neurological systems are summarised in Section 4.

Brief overviews of in vivo biodistribution, pharmacokinetics and metabolism are given in Section 5.

GLP-compliant repeated-dose toxicity studies incorporating immunotoxicity studies were conducted and are presented and discussed in Section 6.

TABLE 1
Summary of main pharmacological and toxicological
characteristics of RNA(LIP) vaccines.

| Category | Features |
|---|---|
| Drug | Liposome complexed mRNAs encoding prostate |
| class | cancer-specific antigens. |
| | In vitro transcription with T7 polymerase using |
| | DNA templates. Batch evaluation by in vitro |
| | translation (potency assay). For IV injection to |
| | prostate cancer patients for neoadjuvant chemo- |
| | therapy of the primary tumor followed by |
| | interval surgery. |
| Lead | The RNA lead structures targeting the prostate |
| structure | cancer antigens are codon-optimized and contain |
| | stabilising untranslated sequences and a modified |
| | cap analog for enhanced stability and translation |
| | capacity. Furthermore, all lead structures consist |
| | of the full length mRNA with stability and trans- |
| | latability enhancing sequence elements and, in |
| | most cases flanked by 5′- and 3′-end coding for |
| | secretory and trans-membrane domains enhancing |
| | processing and presentation of the protein. In |
| | addition, tetanus toxoid-derived helper epitopes |
| | P2 and P16 are fused in frame with the target |
| | antigens. |
| Mode of | Lipoplex formulation protects RNA against |
| action | RNase-mediated degradation enabling IV |
| | administration. |
| | Selective uptake of IV administered and circulating |
| | RNA(LIP) by professional APCs in lymphatic |
| | compartment in particular spleen-resident APCs. |
| | Translation of RNA encoded antigens and pro- |
| | cessing of antigens into peptide epitopes. |
| | Induction of antigen-specific T lymphocytes by |
| | presentation of the RNA encoded epitopes as |
| | peptides complexed with MHC molecule on the |
| | surface of professional APCs in the context of co- |
| | stimulatory signals. |
| | Activation and expansion of tumor-specific T cells. |
| | Signaling of the mRNA through binding to TLRs |
| | and TLR-mediated immunomodulatory effects |
| | leading to cellular activation and induction of pro- |
| | inflammatory cytokines (e.g., IFN-α, IFN-γ, IP-10, |
| | TNF-α, IL-6, and IL-10) enhancing the vaccine effect. |
| Anti- | In vivo eradication of antigen-pulsed target cells. |
| tumoral | Inhibition of tumor growth rate and eradication of |
| activity in | large established tumors after SC tumor challenge in |
| animal | fast-growing syngeneic mouse tumor models. |
| tumor | Increase of median survival time of tumor-bearing |
| models | animals. |
| Relevant | Mice are considered to be a relevant species to |
| animal | measure biological activity and immunological |
| species | effects resulting from vaccines derived from the |
| | RNA(LIP) vaccine platform. Undesired adverse |
| | reactions due to the expected immune-activation |
| | via TLRs and subsequent induction of pro- |
| | inflammatory cytokines can also be assessed in |
| | mice, but have to be complemented with human |
| | in vitro data, as the TLR expression differs between |
| | mice and humans. There is no relevant animal |
| | species to adequately capture potential harmful |
| | effects induced by potentially auto-reactive or |
| | cross-reactive vaccine-induced T-cell responses |
| | given MHC differences when using human |
| | antigen sequences in mice. |
| Pharma- | Rapid degradation of RNA in blood (half-life of |
| cokinetics | about 5 min) and organs within 48 h. Transient |
| | presence of RNA in spleen and liver. Template |
| | DNA residuals do not accumulate or persist in the |
| | gonads. |
| | Transient accumulation of DOTMA in spleen and |
| | liver after repetitive RNA(LIP) application. |
| | DOTMA is cleared from the organs with an |
| | approximate half-life in the order of 6-7 weeks. |
| Safety | No adverse effects were observed in safety |
| pharma- | pharmacology studies (central nervous system |
| cology | (CNS) and respiratory system) in mice. |
| | Cardiovascular safety as indicated by supporting |
| | data from a non-GLP pharmacology study |
| | in cynomolgus monkeys. |
| Toxi- | IV injection of multiple RNA(LIP) was very well |
| cology | tolerated in mice, as shown for a number of mRNA |
| | lead structures assessed in three different repetitive |
| | dose toxicity studies (LPT No. 28864, 30283, |
| | and 30586). |
| | Mild and transient lymphopenia is considered to be |
| | in line with TLR-triggered cytokine induction—an |
| | intended pharmacological effect. |

Section 1: Scientific Foundation

Sequence Features Improving RNA Translation and Intracellular Stability

The RNA vaccine platform has been developed and systematically optimized over 2 decades for safety and efficient induction of antigen-specific CD8+ and CD4+ T-cell responses against the encoded antigens.

As outlined above, the active component (drug substance) is the single-stranded, capped messenger RNA (mRNA), which is translated into protein antigen upon entering antigen presenting cells. The mRNA vaccine format is pharmacologically optimized (Table 2) by (i) a modified cap analog for stabilization of the translationally active RNA, (ii) optimized 5′- and 3′-UTRs for increasing stability and RNA translation, (iii) a signal peptide and MITD sequence that improve MHC class I and II antigen processing, (iv) tetanus toxoid-derived helper epitopes to break immunological tolerance by providing non-specific CD4+ T-cell help, and (v) an elongated free ending poly(A) tail that further enhances RNA stability and translation efficiency.

TABLE 2
RNA structural elements for immune-pharmacological optimization.

| Feature | Effect |
|---|---|
| Optimized cap | Stabilizes and increases amount of translational |
| analog | active RNA |
| 5′-UTR, 3′-UTR | Noncoding sequences that increase RNA stability |
| | and translational efficiency |
| Signal peptide, | Improves MHC class 1 and class II antigen |
| MITD sequence | processing |
| Tetanus toxoid | Provide tumor antigen unspecific CD4+ T-cell help |
| helper epitopes | |
| Elongated free | Noncoding sequence that enhances RNA stability |
| ending | and translational efficiency |
| poly(A) tail | |

Targeting of Antigen-Encoding RNA to Lymphoid-Resident Antigen-Presenting Cells

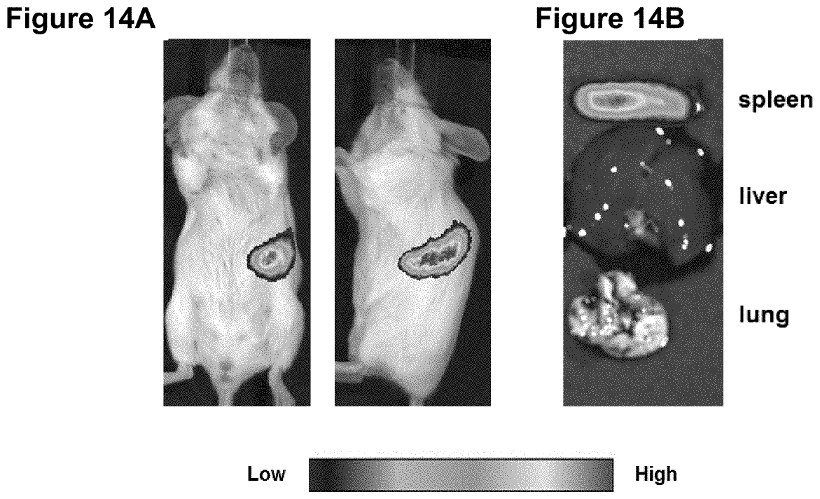

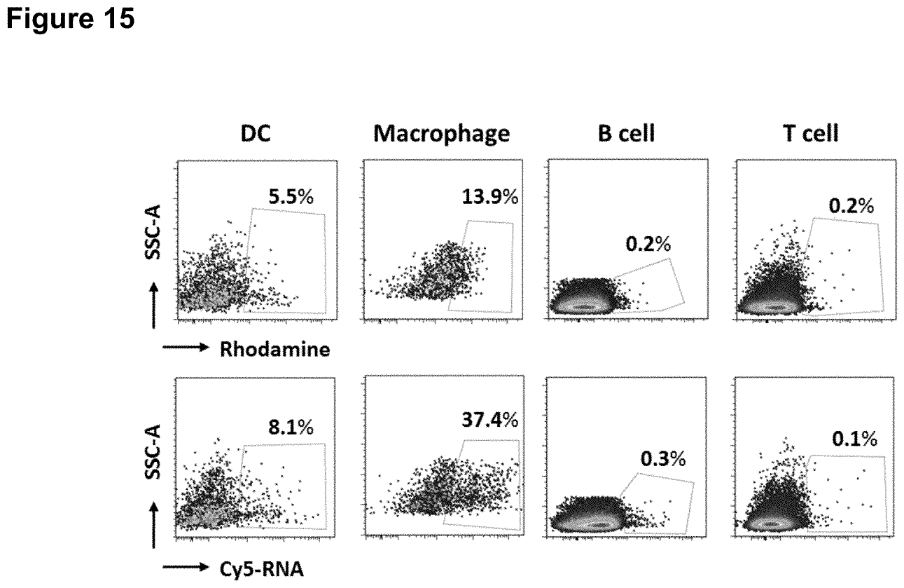

For systemic delivery of RNA to dendritic cells each individual RNA drug product of W_pro1 is formulated to form RNA lipoplexes (RNA(LIP)) that allow for IV administration. The RNA(LIP) formulation was engineered to protect RNA from degradation by plasma RNases and has been optimized for selective delivery of the formulated RNA DPs into antigen-presenting cells (APCs) predominantly residing in the spleen (FIG. 14) and other lymphatic organs where selective uptake of the RNA by dendritic cells and macrophages has been shown (FIG. 15). Once RNA lipoplexes have been taken up by APCs in the spleen, the mRNA is expressed, processed and presented via MHC molecules. This results in the potent induction of antigen-specific CD8+ and CD4+ T-cell responses and T-cell memory which is further supported by the immune stimulatory environment in the spleen induced by TLR-signaling mediated by RNA(LIP).

Induction of Antigen-Specific T-Cell Responses

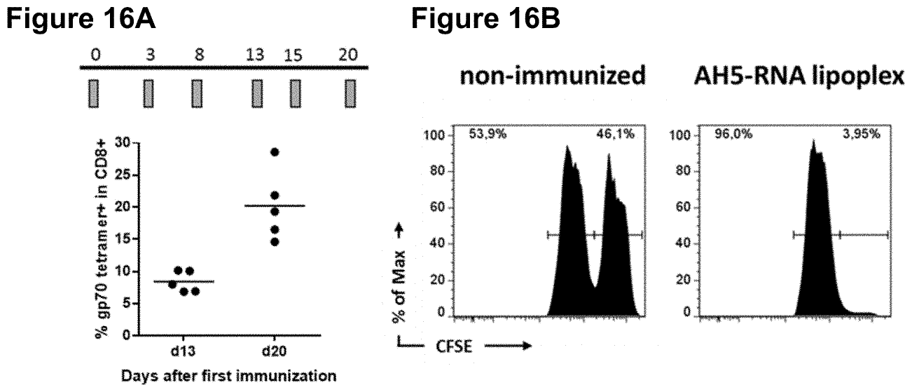

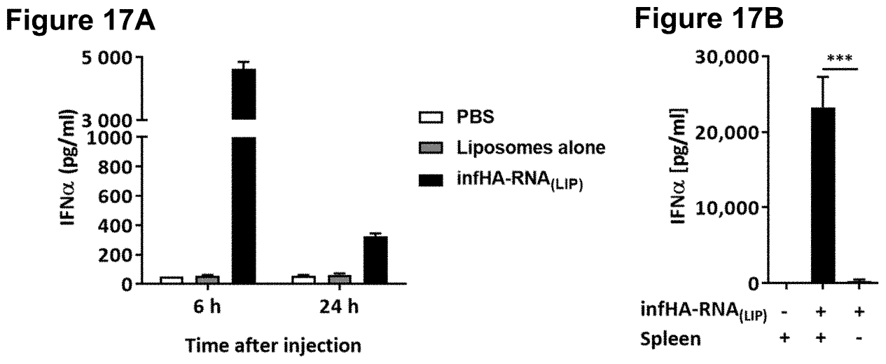

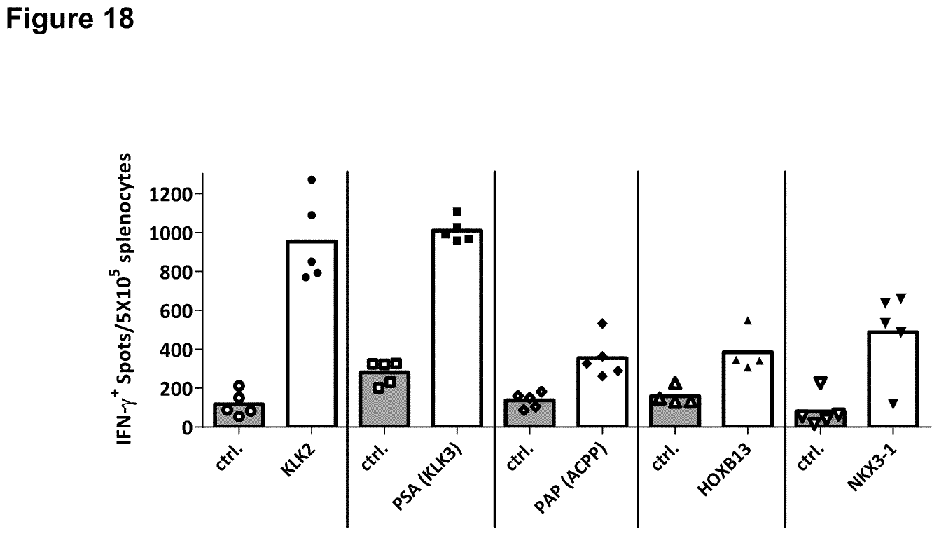

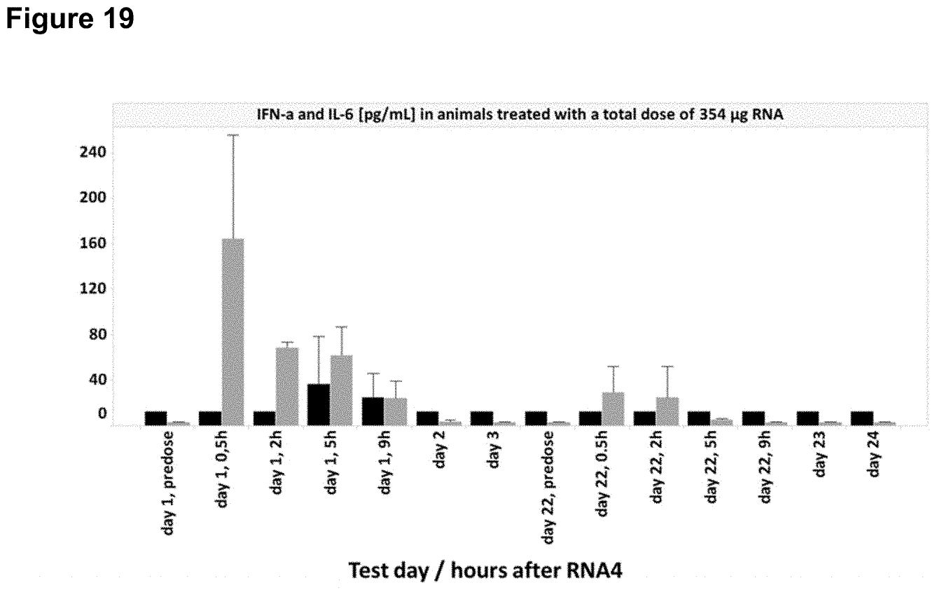

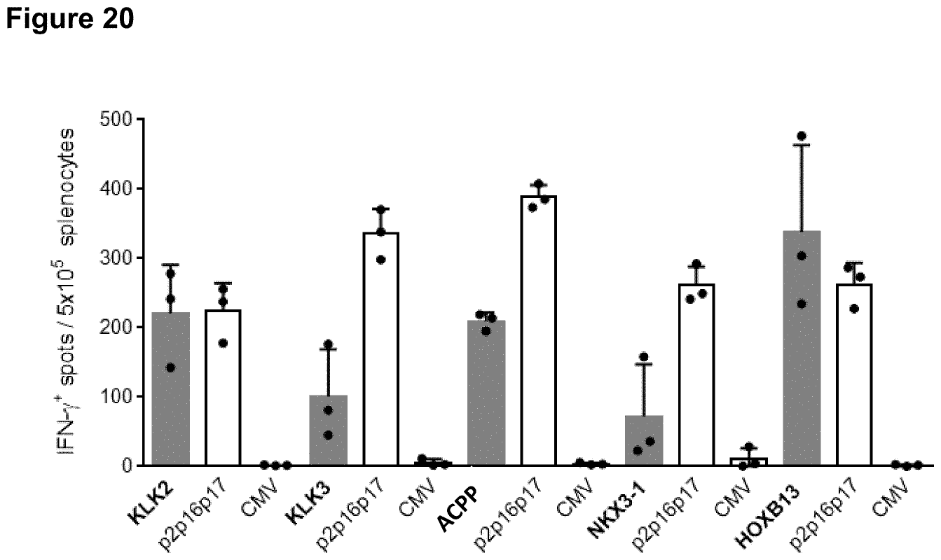

To explore the potency of RNA(LIP) immunization and to study the ability of RNA(LIP) to break tolerance against endogenously expressed antigens, BALB/c mice were immunized with RNA(LIP) coding for the AH5 epitope of the murine leukemia virus (muLV) derived gp70 protein. Repetitive immunizations with AH5-RNA(LIP) led to a profound gp70-specific CD8+ T-cell expansion (FIG. 16A). The CD8+ T cells induced by the vaccination were able to lyse targets in vivo in an antigen-specific manner (FIG. 16B), implying that functional antigen-specific CD8+ T cells with lytic capacities can be induced via RNA(LIP) immunizations.

Induction of Cellular Activation Processes

mRNA is a ligand for human Toll-like receptors (TLRs) and thus able to elicit immunomodulatory effects. Upon cellular uptake of in vitro transcribed (IVT)-RNA, the recognition by TLRs occurs in endosomal compartments, where these receptors are primarily localized. This initiates cascades of signaling events, which eventually lead to the activation and maturation of DCs as has been shown by maturation of splenic DCs after applying the RNA(LIP) vaccine intravenously into mice. Further consequences of these immunomodulatory effects are subsequent activation of splenic T, B, NK cells, and macrophages and the reversible induction of proinflammatory cytokines.

Most importantly, injection with a model RNA(LIP) encoding influenza hemagglutinin HA, displayed a strong induction of IFN-α in mice (FIG. 17A) which was shown in splenectomized mice to originate from spleen (FIG. 17B). Notably, the induction of IFN-α was only shown for RNA(LIP) whereas liposomes alone did not lead to induction of IFN-α in mice. The transient cellular activation and cytokines observed in mice treated with RNA(LIP) vaccines are in line with the findings that RNA vaccines can bind to and triggerTLRs. It has also been shown by others that RNA formulated as particles as well as RNA formulated in aqueous solutions are able to activate TLRs. TLR activation has been shown to induce lymphopenia, leading to an interferon type I-dependent recirculation event of leukocytes. In line with this, activation of dendritic cells as well as other splenic cell populations were severely hampered in TLR7−/− or IFNAR−/− mice. Accordingly, studies in IFNAR−/− mice applying RNA(LIP) did show that the transient hematological changes observed after IV application are primarily mediated by IFN-α downstream effects.

P2P16 Tetanus-Derived Helper Epitopes in W_Pro1

Every W_pro1 RNA DP contains so-called P2P16 amino acid sequences derived from the tetanus toxoid (TT) of Clostridium tetani. These sequences support to overcome self-tolerance mechanisms for efficient induction of immune responses to self-antigens by providing tumor-unspecific T-cell help during priming.

The tetanus toxoid heavy chain includes epitopes that can bind promiscuously to MHC class II alleles and induce CD4+ memory T cells in almost all tetanus vaccinated individuals. In addition, the combination of TT helper epitopes with tumor-associated antigens is known to improve the immune stimulation compared to the application of tumor-associated antigen alone by providing CD4+ mediated T-cell help during priming. To reduce the risk of stimulating CD8+ T cells two peptide sequences known to contain promiscuously binding helper epitopes were selected to ensure binding to as many MHC class II alleles as possible. Based on the data of the ex vivo studies cited above the well-known epitopes P2 (QYIKANSKFIGITEL; TT₈₃₀₋₈₄₄) and P16 (MTNSVDDALINSTKIYSYFPSVISKVNQGAQG; TT₅₇₈₋₆₀₉) were selected.

Pharmacology

The mode of action of RNA(LIP) vaccination relies on (i) the recruitment of antigen-specific T lymphocytes after presentation of peptides derived from the RNA-encoded antigens by professional APCs, and (ii) TLR-mediated immune modulatory effects, which lead to cellular activation and to the induction of pro-inflammatory cytokines such as type I interferons, thereby enhancing the vaccination effects.

In Section 2, we report the activation and expansion of target antigen-specific T cells upon immunization with cancer antigen-encoding RNAs, and anti-tumor effects of antigen RNA(LIP) vaccination.

Extensive in vitro and in vivo studies were conducted to investigate potential secondary effects of the administration of RNA(LIP) vaccines, such as pro-inflammatory cytokine induction and hematological changes which are caused by the intended immunomodulatory effect of RNA(LIP).

In Section 3 a set of studies which assess the degree of cellular activation of human peripheral blood cells (PBMCs) and blood cells in heparinized whole blood is discussed. Moreover, the degree of vaccination-induced cytokine induction and hematological changes in cynomolgus monkeys which were treated with doses above the highest intended clinical dose in humans are shown. Finally, side-by-side comparisons of in vitro cytokine induction in blood samples from human donors and cynomolgus monkeys were conducted and the data generated in these studies was used to support the definition of a safe starting dose for the ongoing clinical trial in malignant melanoma (Lipo-MERIT) and other trials investigating RNA(LIP) immunotherapy.

An overview of nonclinical studies using human blood cells, mice and cynomolgus monkeys as test systems to assess the secondary pharmacodynamics of RNA(LIP) is given in Section 3.

We expect that the observed secondary pharmacodynamic effects observed for liposome formulated RNA are not sequence-dependent and that the presented studies are therefore likewise applicable for other RNA drug products employed.

Summary of Key Findings

In vitro and in vivo studies were conducted to investigate the mode of action of the RNA(LIP) vaccines.

The antigen-coding RNA lead structures RBL038.1, RBL039.1, RBL040.1, RBL041.1, and RBL045.1 induced antigen-specific T-cell responses in vivo in mice expressing human HLA-molecules. Moreover, using murine model antigens anti-tumoral effects of RNA(LIP) vaccination were shown in prophylactic and therapeutic immunization studies in mouse tumor models in vivo.

Furthermore, sequence independent pharmacological effects of RNA(LIP) vaccination were analyzed. RNA(LIP) vaccines induced transient activation of antigen presenting cells leading to subsequent induction of inflammatory cytokines such as IFN-α, IFN-γ, IL-6, and IP-10. Secretion of cytokines including IFN-α was shown to be sourced from spleen cells and was mediated by TLR7 signalling following treatment with RNA(LIP) which is accompanied by transient and fully reversible hematological changes. Notably, RNA(LIP)-mediated cytokine induction and subsequent hematological changes were strongly diminished in mice lacking the interferon-α/β receptor (IFNAR−/−).

Section 2: Primary Pharmacodynamics

Several in vitro and in vivo experiments were performed to prove the immunogenicity of RNA(LIP) vaccination with a number of different mRNAs coding for antigens specific for Melanoma, Breast Cancer, HPV+Head and Neck Cancer, Ovarian Cancer, and other cancer types. The in vivo studies were performed with R&D and GMP-grade material in mice.

Induction of Antigen-Specific T-Cell Responses with the W_Pro1 mRNAs

To obtain more information about the in vivo induction of antigen-specific T cells by the prostate specific antigens KLK2, PSA (KLK3), PAP (ACPP), HOXB13, and NKX3-1, RNA(LIP) products were prepared using RNA of R&D quality and liposomes of GMP-like quality. The RNA(LIP) products were injected intravenously into transgenic mice manipulated to express the human leukocyte antigens HLA-A*0201 and -DRB1*01. Using these mice the priming and expansion of T cells specific for HLA-restricted epitopes can be examined in vivo. A2/DR1 mice were vaccinated four to five times by injection of 30 μg of each antigen RNA complexed with liposomes, followed by isolation of spleen cells (5 days after last vaccination). The priming efficiency of the test items was evaluated by IFN-γ ELISPOT assay after restimulation with bone marrow derived dendritic cells (BMDC) electroporated with the respective mRNA or unrelated control mRNA.

For all antigens, four vaccinations with the RNA(LIP) preparations were sufficient to prime specific T-cell responses in the treated animals (FIG. 18).

These results show that RNA(LIP) vaccines can efficiently induce T-cell responses against W_pro1 antigens in vivo.

The mRNAs used in the studies were manufactured under R&D-conditions. Additional immunogenicity studies in A2/DR1 mice with RBL038.1, RBL039.1, RBL040.1, RBL041.1, and RBL045.1 manufactured under GMP-conditions will be performed. The immunogenicity is expected to be comparable to the results from earlier studies.

In Vivo Anti-Tumoral Activity of Antigen-Specific T Cells Induced by Model Antigen RNAs

Associated with the known challenges to identify murine tumor models, no additional studies addressing the W_pro1 antigens were performed, as no murine homologs of these antigens exist. Instead, we developed suitable tumor models for the ovalbumin-derived SIINFEKL-epitope; human papillomavirus derived E6/E7 antigens and gp70 as models for foreign and a mouse self-antigen for vaccination, respectively.

A summary of the in vivo anti-tumor effects induced by RNA(LIP) is given in Table 3.

TABLE 3
Summary table of in vivo anti-tumor effects of RNA(LIP)

| Study | Method | Result |
|---|---|---|
| Prophylactic | Three cycles of IV | Untreated mice died |
| models | immunization of mice | within 22-28 days after |
| B16F10-OVA | with SIINFEKL- | tumor challenge, all |
| and CT26 | RNA(LIP) or | immunized mice were |
| (STR-30207- | gp70-RNA(LIP) prior | protected in the course |
| 008) | to subcutaneous | of monitoring. |
| | tumor challenge with | |
| | B16F10-OVA or | |
| | CT26, respectively. | |
| Therapeutic | Subcutaneous tumor | Untreated or liposome |
| models | challenge with | treated mice died within |
| B16F10-OVA | B16F10-OVA or | 22-28 days after tumor |
| and CT26 | CT26, followed by | challenge. The immunized |
| (STR-30207- | immunizations with | mice showed a significant |
| 010) | SIINFEKL-RNA(LIP) | delay in tumor growth |
| | or gp70-RNA(LIP) | and shrinkage of tumors |
| | at macroscopic | at times. |
| | tumor sizes. | |
| Therapeutic | Metastatic tumor | Untreated mice developed |
| model | challenge by IV | metastases measured by |
| CT26-IV | injection of CT26-Luc | increasing Luc-signals and |
| metastatic | cells, followed by | macroscopic inspection. |
| model | three immunizations | Treated animals showed |
| (STR-30207- | with gp70- | a reduction of Luc- |
| 012) | RNA(LIP) from | signals after treatment |
| | day 4. Tumor | start and metastases- |
| | growth measurement | free lungs at the |
| | by in vivo imaging | end of the study. |
| | of Luc-expressing | |
| | cells and analysis of | |
| | metastases 17 days | |
| | after tumor | |
| | induction. | |
| Therapeutic | Subcutaneous | Untreated or liposome |
| model | tumor | treated mice died |
| TC-1-SC | challenge with TC-1 | within 22-28 days |
| (STR- | cells, followed by | after tumor challenge. |
| 30207-018) | three immunizations | Immunized mice |
| | with HPV | showed a |
| | E6/E7(LIP) after | strong anti-tumoral |
| | 13 days. | activity with |
| | | shrinkage of tumors |
| | | as big as 1 cm³ |
| | | during treatment and |
| | | complete cure of 30% |
| | | of the animals. |

Section 3: Secondary Pharmacodynamics

To investigate potential secondary effects by administration of RNA lipoplex vaccines, such as the induction of inflammatory cytokines and hematological changes induced by the intended immunomodulatory effect we conducted extensive in vitro and in vivo studies using human blood cells and cynomolgus monkeys as test systems. To our best knowledge the secondary pharmacodynamic effects triggered by TLR and innate immune activation by therapeutic mRNA is not sequence dependent the studies presented in this chapter have been performed with a mixture of equal portions of liposome formulated RBL001.1, RBL002.2, RBL003.1, and RBL004.1 of ATM quality. These RNAs are encoding melanosomal antigens and have been used in clinical trials. The studies have not been repeated with W_pro1 RNA DPs coding for TAAs. As described below, the degree of RNA(LIP) vaccination-induced cytokine induction, hematological changes, complement activation, and clinical chemistry were studied in cynomolgus monkeys that were treated with doses corresponding to the intended doses in humans. Moreover, the degree of cytokine release of human and cynomolgus peripheral blood cells (PBMCs) and blood cells in heparinized whole blood in response to RNA lipoplex treatment was investigated in non-GLP and GLP studies.

In addition, bioinformatic homology searches of the RNA vaccine sequences with the human proteome to exclude potential cross-reactivity of induced T cells were conducted.

Summary of Key Findings

Secondary effects were studied in vivo in cynomolgus monkeys, by examination of cytokine release, hematology, clinical biochemistry, and cardiovascular parameters. Cynomolgus monkeys showed a strong, transient induction of IL-6 and very weak induction of IFN-α at a dose level of 354 μg RNA which we consider as an intended pharmacodynamics effect of the treatment with lipoplexes. This was accompanied by a transient lymphopenia that was recovered after 48 h. There were no indications of cardiovascular toxicities.

Furthermore, cytokine release (IP-10, IFN-α, IFN-γ, TNF-α, IL-1β, IL-2, IL-6, and IL-12) was analyzed in cultured human peripheral blood cells (PBMCs) and in cultured human whole blood, drawn from different donors, following the treatment with RNA(LIP) of ATM quality. In cultured PBMCs, there was a dose-dependent induction of all analytes detectable. However, at doses representing clinical dose levels and above there was an induction of five out of the eight studied markers, namely IP-10, IFN-γ, TNF-α, IL-1β, and IL-6. In cultured whole blood, there was no alteration of cytokine levels detectable regarding the analytes: IFN-γ, TNF-α, IL-1β, IL-2, and IL-12. The chemokine IP-10 (CXCL10) was up-regulated in a dose-dependent manner but not significantly increased as compared to control at dose levels intended for clinical application. Increased induction of IL-6 even though on a low level was detectable in one out of four donors. IFN-α was not significantly increased in any dose compared to diluent control.

In order to obtain further information on the extent of induction of pro-inflammatory cytokines and to test whether the cytokine profile observed in cynomolgus in vivo can be also adequately captured in vitro, additional GLP and non-GLP pharmacodynamic studies were performed using PBMCs and whole blood as test systems. These studies revealed a much better reflection of the in vivo observations in whole blood than in the PBMC test system.

Side-by-side comparison of cytokine secretion in samples from human donors and cynomolgus in whole blood and PBMC test system showed that both species are highly comparable with regard to pro-inflammatory cytokine induction upon incubation with RNA(LIP).

In Vitro Activation of PBMCs and Whole Blood in Healthy Human Donors and Cynomolgus Monkeys

Besides its feature to code for protein antigens, RNA has immunomodulatory effects which originate from its ability to induce cellular activation processes via TLR triggering. On the one hand, the immunomodulatory capacity of RNA vaccine enhances the induction of antigen-specific T-cell responses and should be considered a primary pharmacodynamic effect. On the other hand, too strong or non-specific activation of immune cells may lead to undesired secondary effects and should already be addressed in the preclinical studies.

To study the degree of cellular activation of human blood cells heparinized whole blood, and PBMCs (isolated from heparinized whole blood) from four healthy donors were incubated in vitro with a mixture of equal portions of liposome formulated RNA encoding for melanosomal tumor associated antigens (RBL001.1, RBL002.2, RBL003.1, and RBL004.1) of ATM quality. As the TLR activation by RNA is not sequence dependent the study has not been repeated with W_pro1 RNAs.

RNA(LIP) for each of the four RNA drug products have been prepared separately according to the clinical formulation protocol. In this first study (Study 1, STR-30207-013) a concentration range of 0.014 μg RNA/mL to 3.333 μg RNA/mL which is equivalent to human doses between 0.07 mg and 16.65 mg total RNA was selected (Table 5). As primary endpoint, the activation of cells was determined after 6 h and 24 h by secretion of cytokines (IP-10, IFN-α, IFN-γ, TNF-α, IL-1β, IL-2, IL-6, and IL-12) into the cell culture medium (PBMCs) or plasma (whole blood), respectively.

TABLE 5
Delineation of doses for the in vitro studies based on
intended clinical dose cohorts. The values represent μg
of total RNA per mL whole blood or medium, respectively.
Study Report No.: STR-30207-013

| | μg RNA per | Total dose |
|---|---|---|
| Dose level | ml blood volume | [μg RNA] ^{[1]} |
| 1 | 0.014 | 70 |
| 2 | 0.041 | 205 |
| 3 | 0.123 | 615 |
| 4 | 0.371 | 1,850 |
| 5 | 1.111 | 5,550 |
| 7 | 3.333 | 16,650 |

^{[1]} An average total blood volume of 5 L is assumed.

After incubation of PBMCs with the RNA(LIP) mixture, there was a dose-dependent activation detectable regarding all eight tested analytes, though with high variations in concentration levels. The cytokine response was dominated by five out of the eight selected markers, namely IP-10, IFN-γ, TNF-α, IL-1β, and IL-6 (for summary see Table 4). IFN-α, IL-2, and IL-12 showed only minor induction at highest dose levels tested.

Conversely, no IFN-γ, TNF-α, IL-1β, IL-2, and IL-12 secretion was detectable in the whole blood test system after incubation with RNA(LIP). Here, elevated dose-dependent secretion of IP-10 and IL-6 was observed. For IFN-α only low level baseline secretion was observed that was comparable to diluent control and that was not further elevated by incubation with RNA(LIP) (for summary see Table 4).

In summary, findings in PBMCs showed clear differences compared to whole blood suggesting a higher sensitivity of the test system with PBMCs. Whereas increased cytokine levels for all eight tested analytes were detected in PBMCs, cytokine detection was restricted to IFN-α, IP-10, and IL-6 when using whole blood samples as a test system.

TABLE 4
Summary of results for PBMCs and
whole blood in all donors (study STR-30207-013).

| Cytokine/ | Test system | |
|---|---|---|
| Analyte | PBMCs | Whole blood |
| IFN-α | Elevated secretion detectable | Secretion detectable on a |
| | after 24 h in all donors | very low level in some |
| | Dose-dependent induction | samples of 3/4 donors |
| | Values on a low level and not | No distinct elevation |
| | elevated remarkably in doses | detectable compared to |
| | 0.014-0.37 μg/mL of RNA | control in any dilution |
| IP-10 | Elevated secretion detectable | Elevated secretion detectable |
| | after 24 h in all donors | after 24 h in all donors |
| | Dose-dependent induction | Dose-dependent induction |
| | In 4/4 donors elevated levels | In 2/4 donors elevated levels |
| | detectable at 0.37 μg/mL of RNA | detectable at 0.37 μg/mL |
| | In 3/4 donors elevated levels | |
| | detectable at 0.12 μg/mL of RNA | |
| IL-6 | Elevated secretion detectable | Elevated secretion detectable |
| | after 6 h and 24 h in all donors | on a very low level only in |
| | Dose-dependent induction | highest dose in 2/4 donors |
| | In 4/4 donors elevated levels | |
| | detectable at 0.37 μg/mL of RNA | |
| | In 3/4 donors elevated | |
| | levels detectable at | |
| | 0.12 μg/mL of RNA after 24 h | |
| IFN-γ | Elevated secretion detectable | No elevated secretion |
| | after 24 h in all donors | detectable |
| | Dose-dependent induction | |
| | In 4/4 donors elevated levels | |
| | detectable at 0.37 μg/mL of RNA | |
| TNF-α | Elevated secretion detectable | No elevated secretion |
| | after 6 h and 24 h in all donors | detectable |
| | Dose-dependent induction | |
| | In 4/4 donors elevated | |
| | levels detectable at 0.37 | |
| | μg/mL of RNA only after 6 h | |
| IL-β | Elevated secretion detectable | No elevated secretion |
| | after 6 h and 24 h in all donors | detectable |
| | Dose-dependent induction | |
| | In 4/4 donors elevated levels | |
| | detectable at 0.37 μg/mL of RNA | |
| | In 2/4 donors elevated | |
| | levels detectable at | |
| | 0.12 μg/mL of RNA after 24 h | |
| IL-2 | Elevated secretion detectable | No elevated secretion |
| | after 6 h and 24 h in all donors | detectable |
| | Dose-dependent induction | |
| | Values on a low level and | |
| | not elevated in doses | |
| | 0.014-0.37 μg/mL of RNA | |
| IL-12 | Elevated secretion detectable | No elevated secretion |
| | after 24 h in all donors | detectable |
| | Dose-dependent induction | |
| | In 2/4 elevated levels detectable | |
| | at 0.37 μg/mL of RNA | |

To further study the cytokine release of human cells in response to RNA(LIP) in vitro and to compare and classify the in vivo data from the mouse immunotoxicity studies (see below) and the cynomolgus study (see below) an additional GLP-compliant in vitro study was performed at an external CRO (LPT No. 31031). Major aim of this study was to confirm (i) whether findings in cynomolgus monkeys are comparable to human and (ii) which test system better reflects the cytokine response pattern observed in cynomolgus monkeys in vivo. The study LPT No. 31031 was designed as follows: the in vitro induction of pro-inflammatory cytokines in healthy human donors and cynomolgus monkeys was tested in two test systems, namely PBMCs and whole blood. The same dose-range and dosage-steps of RNA(LIP) as in study No. STR-30207-013 was tested. Test item was again a mixture of separately prepared liposome formulated RBL001.1, RBL002.2, RBL003.1, and RBL004.1 RNAs of ATM quality. As mentioned above the data generated with these IVT-RNAs also account for RNA DPs coding for TAAs, because TLR-activation is RNA sequence independent. In total, samples of four individuals of each species were analyzed. As primary endpoint, the activation of cells was determined after 6 h, 24 h, and 48 h by secretion of pro-inflammatory cytokines into the cell culture medium (PBMCs) or plasma (whole blood), respectively.

The cytokine responses observed are summarized in Table 6 for the whole blood test system and in Table 7 for the PBMC test system, respectively.

TABLE 6
Summary of cytokine responses in the whole blood test system.
Test system: Whole blood

| Cytokine/ | |
|---|---|
| Analyte | Result |
| TNF-α | Elevated secretion detectable in 4/4 monkeys and 4/4 humans |
| | Maximum absolute cytokine levels at highest dose: |
| | Monkeys: 1/4: <100 pg/mL; 1/4: 100-500 pg/mL; 2/4: |
| | 500-1,000 pg/mL |
| | Humans: 1/4: <100 pg/mL; 3/4: 500-1,000 pg/mL |
| IFN-γ | Elevated secretion detectable in 2/4 monkeys only |
| | Maximum absolute cytokine levels at highest dose: |
| | Monkeys: 2/4 <100 pg/mL |
| IL-6 | Elevated secretion detectable in 4/4 monkeys and 4/4 humans |
| | Maximum absolute cytokine levels at highest dose: |
| | Monkeys: 2/4: 100-500 pg/mL; 2/4: 500-1,000 pg/mL |
| | Humans: 1/4: 500-1,000 pg/mL; 3/4: 1,000-5,000 pg/mL |
| IP-10 | Elevated secretion detectable in 4/4 humans only |
| | Maximum absolute cytokine levels at highest dose: |
| | Humans: 4/4: 500-1,000 pg/mL |
| IL-β | Elevated secretion detectable in 4/4 monkeys and 4/4 humans |
| | Maximum absolute cytokine levels at highest dose: |
| | Monkeys: 2/4: <100 pg/mL; 2/4: 100-500 pg/mL |
| | Humans: 2/4: <100 pg/mL; 2/4: 100-500 pg/mL |
| IL-12 | Elevated secretion detectable in 4/4 humans only |
| | Maximum absolute cytokine levels at highest dose: |
| | Humans: 3/4: 100 pg/mL; 1/4: 100-500 pg/mL |
| IL-2 | no elevated secretion in any monkey or human detectable |

TABLE 7
Summary of cytokine responses in the PBMC
test system (LPT No. 31031).
Test system: PBMCs

| Cytokine/ | |
|---|---|
| Analyte | Result |
| TNF-α | Elevated secretion detectable in 4/4 monkeys and 4/4 humans |
| | Maximum absolute cytokine levels at highest dose: |
| | Monkeys: 4/4: 1.000-5,000 pg/mL |
| | Humans: 4/4: 1.000-5,000 pg/mL |
| IFN-γ | Elevated secretion detectable in 4/4 monkeys and 4/4 humans |
| | Maximum absolute cytokine levels at highest dose: |
| | Monkeys: 2/4: 100-500 pg/mL; 2/4: 500-1,000 pg/mL |
| | Humans: 2/4: 1,000-5,000 pg/mL; 2/4: 5,000-10,000 pg/mL |
| IL-6 | Elevated secretion detectable in 4/4 monkeys and 4/4 humans |
| | Maximum absolute cytokine levels at highest dose: |
| | Monkeys: 1/4: 1,000-5,000 pg/mL; 3/4: 5,000-10,000 pg/mL |
| | Humans: 2/4: 5,000-10,000 pg/mL; 2/4: 10,000-15,000 pg/mL |
| IP-10 | Elevated secretion detectable in 4/4 humans only |
| | Maximum levels at highest dose: |
| | Humans: 4/4: 100-500 pg/mL |
| IL-β | Elevated secretion detectable in 4/4 monkeys and 4/4 humans |
| | Maximum absolute cytokine levels at highest dose: |
| | Monkeys: 4/4: 1,000-5,000 pg/mL |
| | Humans: 4/4: 1,000-5,000 pg/mL |
| IL-12 | Elevated secretion detectable in 4/4 monkeys and 4/4 humans |
| | Maximum absolute cytokine levels at highest dose: |
| | Monkeys: 4/4: <100 pg/mL |
| | Humans: 1/4: 100-500 pg/mL; 3/4: 500-1,000 pg/mL |
| IL-2 | no elevated secretion in any monkey or human detectable |

Table 8 shows the data generated in study LPT No. 31031 in which whole blood from four cynomolgus monkeys and four healthy donors were analyzed after 6 h and 24 h incubation with six different doses of RNA(LIP). Analysis was focused on the pro-inflammatory cytokines TNF-α, IL-6, and IFN-γ as they were pre-dominantly up-regulated in human PBMCs in study No. STR-30207-013. As shown, the cytokine responses in vitro in the two species were highly comparable. For IL-6 a 122-fold induction in cynomolgus monkeys and a 108-fold induction in healthy donors, respectively, was observed after 24 h incubation. Only low levels of TNF-α could be detected in both species at the highest dose level. Very low IFN-γ induction was observed only at the highest dose level in cynomolgus monkeys after 24 h incubation.

Most importantly, strong test item-related cytokine induction of these three pro-inflammatory cytokines was only observed at dose levels >5,500 μg which is above the highest intended dose level of 100 μg in patients and which is >100-fold higher as the planned dose for the initial vaccination cycle (=50 μg RNA). Notably, the results from the healthy donors confirmed the findings from the in vitro study STR-30207-013 and the cynomolgus cytokine response pattern observed in the whole blood test system resembled the findings from the in vivo study LPT No. 29928 in which only IL-6 could be detected in cynomolgus monkeys treated with RNA(LIP) (see below).

Table 9 shows the data generated in study LPT No. 31031 in which PBMCs from four cynomolgus monkeys and four healthy donors were analyzed after 6 h and 24 h incubation with six different doses of RNA(LIP). Induction of IL-6 and TNF-α was comparable in human and cynomolgus PBMCs with regard to (i) absolute amounts of cytokines induced (less than factor 2 differences between species), (ii) kinetics (early induction of IL-6 and TNF-α after 6 h), and (iii) dose level of RNA(LIP) that led to cytokine induction. IFN-γ was detected in PBMCs from both species treated with intermediate doses of RNA(LIP) only after 24 h of RNA(LIP) stimulation albeit to higher extent in humans. In sum, the cytokine profiles induced by RNA(LIP) in PBMCs were comparable across species with respect to IL-6 and TNF-α. The results obtained in this study suggest that cynomolgus monkeys are a relevant species to assess RNA(LIP)-mediated cytokine induction and that human PBMCs constitute the more sensitive system for capturing IFN-γ induction.

TABLE 8
In vitro induction of the pro-inflammatory cytokines
IL-6, TNF-α, and IFN-γ in cynomolgus monkeys and
healthy human donors in the whole blood test system.
The table shows data generated in study LPT No. 31031:
cytokine levels (pg/mL) of IL-6 (upperpart), TNF-α
(middle part), and IFN-γ (lower part) detected after
incubation of whole blood-with different doses of
RNA(LIP) The red color code indicates the height of
the cytokine level with the darker red indicating the
higher cytokine levels. The first column indicates the
total dose levels applied in clinical settings. The
second column indicates the amount of RNA used
in the in vitro test system assuming a 5 L blood voume.

| | Total | μg RNA | 6 h | | 24 h | |
|---|---|---|---|---|---|---|
| | Dose | tested | Cyno- | | Cyno- | |
| | [μg | in | molgus | | molgus | |
| | RNA] | assay ^{[1]} | Human | | Human | |
| IL-6 | | 0 | 4 | 10 | 4 | 13 |
| (pg/mL) | 7.2 | | * | * | * | * |
| | 14.5 | | * | * | * | * |
| | 29 | | * | * | * | * |
| | 50.4 | | * | * | * | * |
| | 72.8 | 70 | 4 | 21 | 4 | 15 |
| | 200 | 205 | 4 | 9 | 4 | 22 |
| | | 615 | 4 | 43 | 4 | 35 |
| | | 1,850 | 4 | 17 | 13 | 51 |
| | | | * | * | * | * |
| | | 5,550 | 7 | 238 | 25 | 415 |
| | | 16,650 | 139 | 807 | 488 | 1,408 |
| TNF-α | | 0 | 5 | 9 | 5 | 9 |
| (pg/mL) | 7.2 | | * | * | * | * |
| | 14.5 | | * | * | * | * |
| | 29 | | * | * | * | * |
| | 50.4 | | * | * | * | * |
| | 72.8 | 70 | 5 | 9 | 5 | 9 |
| | 200 | 205 | 5 | 9 | 5 | 9 |
| | | 615 | 5 | 14 | 5 | 9 |
| | | 1,850 | 7 | 11 | 5 | 9 |
| | | | * | * | * | * |
| | | 5,550 | 18 | 73 | 5 | 9 |
| | | 16,650 | 570 | 624 | 77 | 28 |
| IFN-γ | | 0 | 3 | 4 | 3 | 4 |
| (pg/mL) | 7.2 | | * | * | * | * |
| | 14.5 | | * | * | * | * |
| | 29 | | * | * | * | * |
| | 50.4 | | * | * | * | * |
| | 72.8 | 70 | 3 | 4 | 4 | 4 |
| | 200 | 205 | 3 | 4 | 6 | 4 |
| | | 615 | 3 | 4 | 4 | 4 |
| | | 1,850 | 3 | 4 | 5 | 4 |
| | | | * | * | * | * |
| | | 5,550 | 3 | 4 | 8 | 4 |
| | | 16,650 | 3 | 4 | 24 | 6 |

* = no data collected.
^{[1]} An average total blood volume of 5 L is assumed.

TABLE 9
In vitro induction of the pro-inflammatory cytokines
IL-6, TNF-α, and IFN-γ in cynomolgus monkeys
and healthy human donors in the PBMC test system.
The table shows data generated in study LPT No.
31031: cytokine levels (pg/mL) of IL-6 (upper part),
TNF-a (middle part), and IFN-y (lower part) detected
after incubation of PBMCs with different doses of
RNA(LIP). The red color code indicates the height of
the cytokine level with the darker red indicating the
higher cytokine levels. The first column indicates the
total dose levels applied in clinical settings. The second
column indicates the amount of RNA used in the
in vitro test system assuming a 5 L blood volume.

| | Total | μg RNA | 6 h | | 24 h | |
|---|---|---|---|---|---|---|
| | Dose | tested | Cyno- | | Cyno- | |
| | [μg | in | molgus | | molgus | |
| | RNA] | assay ^{[1]} | Human | | Human | |
| IL-6 | | 0 | 9 | 11 | 29 | 9 |
| (pg/mL) | 7.2 | | * | * | * | * |
| | 14.5 | | * | * | * | * |
| | 29 | | * | * | * | * |
| | 50.4 | | * | * | * | * |
| | 72.8 | 70 | 30 | 23 | 407 | 109 |
| | 200 | 205 | 84 | 33 | 1,142 | 367 |
| | | 615 | 268 | 71 | 2,500 | 799 |
| | | 1,850 | 771 | 182 | 5,706 | 2,817 |
| | | | * | * | * | * |
| | | 5,550 | 1,451 | 1,066 | 6,484 | 4,700 |
| | | 16,650 | 2,047 | 2,973 | 5,419 | 9,696 |
| TNF-α | | 0 | 9 | 11 | 29 | 9 |
| (pg/mL) | 7.2 | | * | * | * | * |
| | 14.5 | | * | * | * | * |
| | 29 | | * | * | * | * |
| | 50.4 | | * | * | * | * |
| | 72.8 | 70 | 45 | 17 | 88 | 100 |
| | 200 | 205 | 110 | 58 | 282 | 337 |
| | | 615 | 279 | 191 | 646 | 740 |
| | | 1,850 | 814 | 601 | 1,207 | 1,612 |
| | | | * | * | * | * |
| | | 5,550 | 1,596 | 1,688 | 1,632 | 2,167 |
| | | 16,650 | 2,316 | 3,472 | 1,736 | 3,684 |
| IFN-γ | | 0 | 3 | 4 | 3 | 4 |
| (pg/mL) | 7.2 | | * | * | * | * |
| | 14.5 | | * | * | * | * |
| | 29 | | * | * | * | * |
| | 50.4 | | * | * | * | * |
| | 72.8 | 70 | 3 | 4 | 4 | 19 |
| | 200 | 205 | 3 | 4 | 72 | 213 |
| | | 615 | 3 | 4 | 175 | 943 |
| | | 1,850 | 3 | 4 | 374 | 1,970 |
| | | | * | * | * | * |
| | | 5,550 | 3 | 4 | 247 | 3,416 |
| | | 16,650 | 3 | 20 | 56 | 2,674 |

* = no data collected.
^{[1]} An average total blood volume of 5 L is assumed.

When comparing the results found in the whole blood and the PBMC test system it became clear that that pro-inflammatory cytokines in the PBMC test system was generally broader, reached higher absolute values and started at lower dose levels as compared to the whole blood test system.

In this most sensitive in vitro test system, a steep increase of cytokine levels as measured after 24 h was observed at dose ranges between 615 μg to 1,850 μg RNA for IL-6, 1,850 μg to 5,550 μg RNA for IFN-γ, and 5,550 μg to 16,650 μg RNA for TNF-α. Even for IL-6 which was the most sensitive cytokine marker in the in vitro system, the intended starting dose of 25 μg is 25-times lower as the dose level that mark the initiation of strong in vitro cytokine induction. In addition to the GLP study LPT No. 31031, a non-GLP in vitro study (Report_RB_14_001_B) with a similar experimental setup was conducted testing samples from three individuals per species in which similar observations were made confirming the results from the GLP study (data not shown). Taken all studies together, the findings underline (i) the comparability of both species, cynomolgus monkeys and human, regarding the stimulation of cells after incubation with the test item. Moreover, these observations showed (ii) that the whole blood test system reflects the in vivo situation more closely than the PBMCs. In the whole blood test system cytokine induction was generally less pronounced and observed only in the highest dose groups, and the predominant induction of IL-6 resembles the findings of a cynomolgus in vivo study (see below).

We acknowledge the more pronounced findings in PBMCs which are considered to be artificial but also more sensitive in vitro test system and therefore integrated the results from this more sensitive test system in the strategy to define a safe initial starting dose.

In Vivo Testing of Secondary Pharmacology in Cynomolgus Monkeys

In order to understand the kinetics and the correlation of secondary effects of RNA(LIP) with cytokine expression more precisely, a non-GLP study was performed in male cynomolgus monkeys (see Table 10 for the treatment schedule and doses and Table 11 for the detailed study design and the amounts of all formulation components). The animals (two males per dose) in groups 1 to 5 were treated with the four melanosomal RNA(LIP) vaccines RBL001.1, RBL002.2, RBL003.1, and RBL004.1 (ATM quality) and control solutions were given subsequently as slow bolus injections (approx. 10 s), with an interval of 30 min between each injection (i.e., the last injection was given after 1.5 h). The results of this study should also apply for W_pro1 injection, as the secondary effects are not sequence dependent. Doses up to 42-fold above the highest clinical dose were tested within the study. In addition, animals of the dose group 6 received a single dose of 4×3.6 μg RNA on day 22 after having received a single dose of 4×88.6 μg RNA on day 1.

TABLE 10
Study schedule and doses in relation to the intended
doses in patients (LPT No. 29928).
Treatment: animals 1-10 (groups 1-5) were treated 5
times with four subsequent injections of NaCl (saline)
(group 1), liposomes of the same dose as high dose
animals (group 2), and RNA(LIP)1-4 (ATM quality,
groups 3-5). Animals in group 6 received a single
treatment with 4 × 88.6 μg (354 μg of RNA in total)
on test day 1, followed by a single treatment with
4 × 3.6 μg (14.4 μg of RNA in total) on test day 22.
Doses: doses are shown in total RNA amount in
mg/kg body weight and as the total RNA dose
(μg per individual, patients are estimated with
a body weight of 70 kg).

| | | | Dose | Dose |
|---|---|---|---|---|
| | Animal | Application | [mg/kg | total |
| Group | ID | days | b.w.] | RNA [μg] |
| 1 | 1, 2 | Days 1, 4, | NaCl | NaCl |
| | | 8, 15, 22 | | |
| 2 | 3, 4 | Days 1, 4, | Lipo- | Lipo- |
| | | 8, 15, 22 | somes | somes |
| 3 | 5, 6 | Days 1, 4, | 0.0086 | 43 |
| | | 8, 15, 22 | | |
| 4 | 7, 8 | Days 1, 4, | 0.0256 | 128 |
| | | 8, 15, 22 | | |
| 5 | 9, 10 | Days 1, 4, | 0.0708 | 354 |
| | | 8, 15, 22 | | |
| 6 | 11, 12 | Day 1 | 0.0708 | 354 |
| 6 | 11, 12 | Day 22 | 0.029 | 14 |

TABLE 11
Design of a pharmacodynamic study in cynomolgus
monkeys (LPT No. 29928).
Design of a pharmacodynamic study of RBL001.1,
RBL002.2, RBL003.1, and RBL004.1 after IV
administration to cynomolgus monkeys.

| Test Item | | RBL001.1, RBL002.2, RBL003.1, and | | | |
|---|---|---|---|---|---|
| | | RBL004.1 RNA(LIP) | | | |
| Administration | | 5 administrations of on day 1, 4, 8, 15, | | | |
| Route | | and 22 except for group 6 | | | |
| | | IV bolus into the vena cephalica of the left | | | |
| | | or right arm Bolus injection (approx. 10 s) | | | |
| | | of RBL001.1, RBL002.2, RBL003.1, and | | | |
| | | RBL004.1 with 30 min intervals between | | | |
| | | each of the RNA(LIP) | | | |
| Dose groups | | 1. NaCl control ^{[1]} | | | |
| | | 2. liposome control | | | |
| | | 3. low dose | | | |
| | | 4. mid dose | | | |
| | | 5. high dose | | | |
| | | 6. single high dose on day 1 followed | | | |
| | | by a recovery period additional very | | | |
| | | low dose on day 22 | | | |
| | Application | RNA | Lipids | DOTMA | DOPE |
| Doses | days | [μg] | [μg] | [μg] | [μg] |
| Group 1 | 1, 4, 8, 15, 22 | — | — | — | — |
| Group 2 | 1, 4, 8, 15, 22 | — | 4 × 181 | 4 × 116 | 4 × 65 |
| Group 3 | 1, 4, 8, 15, 22 | 4 × 10.75 | 4 × 22 | 4 × 14 | 4 × 8 |
| Group 4 | 1, 4, 8, 15, 22 | 4 × 32 | 4 × 65 | 4 × 42 | 4 × 23 |
| Group 5 | 1, 4, 8, 15, 22 | 4 × 88.6 | 4 × 181 | 4 × 116 | 4 × 65 |
| Group 6 | 1 | 4 × 88.6 | 4 × 181 | 4 × 116 | 4 × 65 |
| Group 6 | 22 | 4 × 3.6 | 4 × 8 | 4 × 5 | 4 × 3 |

Group size 2 male animals/group, 12 animals in total
^{[1]} NaCl is considered as being the most appropriate control group. In contrast to liposome formulated RNA that forms RNA(LIP) of a defined size and charge, pure liposomes applied in group 2 differ significantly in terms of physical characteristics, e.g., charge and structure leading to different pharmacological properties and changed biodistribution in vivo.

Clinical Observation

Overall, the treatment was very well tolerated. There were no abnormal signs of intolerances noted in any animal regarding local and systemic tolerance observations (including behavior, external appearance, feces, mortality, body weight, and food and water uptake).

Cytokine Analysis

Cytokine release into plasma was studied for IFN-α, IFN-γ, TNF-α, IL-1S, IL-2, IL-6, IL-10, IL-12p70, and IP-10 in two kinetics after the 1^{st} and after the 5^{th} injection, at predose, 0.5, 2, 5, 9, 24, and 48 h after completion of the treatment (i.e., after completing the injection cycle of all 4 RNA(LIP) products).

At the tested doses, only IL-6 showed a dose-dependent and test item-related induction. Cmax levels were reached at 30 min after completion of the treatment, and were back to predose levels after 24 h (FIG. 19). Animal 11 (group 6) was an outlier showing a very strong response and had IL-6 peak levels of 1,071 μg/mL, which was approximately 5× higher than in other animals of the same dose group. Of note, IL-6 induction was much lower after the 5^{th} treatment, suggesting an adaption effect for IL-6 in monkeys.

IFN-α induction was observed only at very low levels in animals of the high dose group 6, reaching the highest levels after 5 h, which were back to predose levels after 24 h (FIG. 19). In contrast to observations in cultured human cells and in vivo in mice IP-10 induction was not observed in monkeys. It remains open why IP-10 was not observed in this study, since IP-10 induction has been observed in monkeys after TLR activation agonists as reported by others. Other tested cytokines (IFN-γ, TNF-α, IL-1β, IL-2, IL-10, and IL-12p70) were not changed. Liposomes alone did not have an effect on cytokine release.

Hematology

Standard hematology parameters were tested after the 1^{st} and after the 5^{th} injection at predose, 5, 9, 24, and 48 h after completion of the treatment (2 h were additionally included after the 5^{th} dose). In addition, hematology was tested daily from test day 4 to day 12, and 1 and 3 weeks after the last dosing.

A transient decrease of lymphocytes and a transient increase of neutrophils were found as test item-related findings in a dose-dependent manner. Lymphocytes dropped very quickly at 5 h after completion of the treatment up to 5-fold in high dose animals (lowest amount approx. 1,000 lymphocytes/μL in animals of group 6). The effect was transient and recovered in approximately 48 h. Of note, lymphocyte depletion was also observed in animals of the liposome group to a lower extend but not in the NaCl control group (Table 12). There was no adaption effect as observed for the IL-6 induction.

Increase of neutrophils was also observed in the NaCl control group due to the treatment, however, a significant difference was observed in groups 3 to 6, when compared to the control. The maximum effects were observed 10 h after the treatment and were 44%, 34%, 89%, and 91% versus control in group 3, 4, 5, and 6, respectively.

Treatment related, transient effects (also in NaCl group) were observed for eosinophils, leucocytes, and reticulocytes (probably due to the constant blood sampling).

TABLE 12
Results for absolute lymphocyte counts [1,000/μL] in cynomolgus monkeys
(mean values of n = 2).

| | Test | | | | | | | |
|---|---|---|---|---|---|---|---|---|
| | day/hours | | | | | | Group 6: | Group 6: |
| | after | Group 1: | Group 2: | Group 3: | Group 4: | Group 5: | 354 μg_ | 14.4 μg_ |
| Dosing | RNA4 | NaCl | liposome | 43 μg | 128 μg | 354 μg | single | single |
| 1 | day 1, | 6.170 | 9.130 | 6.190 | 6.490 | 7.060 | 5.700 | n.d. |
| | predose | | | | | | | |
| | day 1, 5 h | 4.845 | 3.295 | 2.095 | 2.665 | 1.490 | 1.065 | n.d. |
| | day 1, 9 h | 8.605 | 6.365 | 4.250 | 4.200 | 1.850 | 1.78 | n.d. |
| | day 2 | 5.165 | 8.950 | 4.065 | 4.300 | 4.190 | 3.37 | n.d. |
| | day 3 | 5.725 | 9.075 | 4.745 | 5.375 | 5.250 | 5.425 | n.d. |
| 2 | day 4 | 5.775 | 8.185 | 5.175 | 5.265 | 5.530 | 5.87 | n.d. |
| | day 5 | 5.565 | 8.850 | 4.480 | 4.170 | 4.600 | 5.845 | n.d. |
| | day 6 | 5.865 | 8.560 | 5.590 | 6.660 | 7.200 | 5.495 | n.d. |
| | day 7 | 7.090 | 9.645 | 6.225 | 6.780 | 8.660 | 6.885 | n.d. |
| 3 | day 8 | 5.980 | 10.555 | 6.020 | 6.955 | 8.775 | 6.325 | n.d. |
| | day 9 | 4.870 | 7.870 | 3.465 | 4.260 | 4.960 | 5.430 | n.d. |
| | day 10 | 5.495 | 8.125 | 4.350 | 5.885 | 7.025 | 5.400 | n.d. |
| | day 11 | 6.360 | 8.325 | 4.725 | 6.045 | 8.555 | 5.640 | n.d. |
| | day 12 | 5.305 | 7.555 | 5.290 | 5.155 | 7.950 | 5.815 | n.d. |
| 4 | day 15 | 8.180 | 11.030 | 9.540 | 9.170 | 9.855 | n.d. | n.d. |
| | day 16 | 4.765 | 7.320 | 2.880 | 3.365 | 3.490 | n.d. | n.d. |
| | day 19 | 5.545 | 8.230 | 5.215 | 5.485 | 8.035 | 6.205 | n.d. |
| 5 | day 22, 2 h | 4.360 | 4.385 | 2.055 | 2.645 | 2.120 | n.d. | 2.955 |
| | day 22, 5 h | 5.525 | 5.225 | 2.955 | 3.325 | 2.445 | n.d. | 3.485 |
| | day 22, 9 h | 8.560 | 12.525 | 5.490 | 4.650 | 3.695 | n.d. | 4.625 |
| | day 23 | 4.645 | 7.655 | 3.505 | 3.720 | 4.685 | n.d. | 4.350 |
| | day 24 | 5.780 | 8.360 | 4.760 | 4.50 | 6.270 | n.d. | 5.505 |
| | day 30 | 6.120 | 8.190 | 5.265 | 6.370 | 6.360 | n.d. | 5.615 |

Complement Activation

C3a was measured at predose, 0.5, 2, 5, 9, 24, and 48 h after completion of the 1^{st} and 5^{th} treatment, 1 and 3 weeks after the last dosing. No test item-related changes were observed and all values were regarded to be within the normal range of biological variability.

Clinical Chemistry

Standard parameters were tested at predose, 24 h after each dosing and additionally 4 days after the 3^{rd} and 4^{th} dosing, and 1 and 3 weeks after the last dosing.

No test item-related influence was rated on the biochemical parameters for the animals of the liposome-treated group and for the test item-treated animals in comparison to the control animals and/or background data available at the CRO conducting the study. In part, the data show some scatter due to the small number of animals employed per group.

No test item-related changes were noted for the serum levels of bile acids, bilirubin, cholesterol, creatinine, glucose, phosphate, total protein, triglycerides, urea, calcium, chloride, potassium, and sodium, and for the serum proteins (albumin, globulins, and the albumin/globulin ratio).

The serum enzyme activities of alanine aminotransferase (ALAT), alkaline phosphatase (aP), aspartate aminotransferase (ASAT), lactate dehydrogenase (LDH), alpha-amylase, creatine kinase (CK, including isoforms CK-BB, CK-MB, and CK-MM), gamma-glutamyl transferase (gamma-GT), and glutamate dehydrogenase (GLDH) were considered to range within the limits of normal biological variability.

On test day 23, high values were noted for the enzyme activities of LDH, alpha-amylase, and CK for animal no. 11 treated with 4×3.5 μg RNA/animal on test day 22. However, these changes are considered as stress-related due to restraining of the monkey in the infusion chair and not test item-related.

Though rated as not test item-related, the slight changes for CK were evaluated in more detail. A differential analysis of the CK isoenzymes CK-BB, CK-MB, and CK-MM revealed that the increased CK activity noted for individual animals of groups 4, 5, or 6 in comparison to the control animals on test days 9, 16, or 23 was mainly due to an increase of the CK-MM fraction. Generally, no increases were noted for the CK-BB and CK-MB, hence confirming that the increase in overall CK-levels was stress-related.

Cardiovascular Examination

ECG and blood pressure measurements did not show any effects on the cardiovascular system.

Sequence Homology Screen Between the RNA DPs Coding for TAA and the Human Proteome

All mRNA sequences used in the W_pro1 approach are fused in-frame to up to two flanking glycine/serine (GS) rich linker sequences. The suture points of these fusions may create new antigenic fusion proteins or peptides, which could potentially raise an unwanted autoimmune response, in case they are homologous to human proteins. Therefore, it was determined whether the suture points associated with the linker sequences and the antigen possess sequence homology to known human proteins by BLASTp based homology search against a database of established human proteins.

The fusion protein sequences to be analyzed were disassembled into smaller peptide sequences by using a sliding window with lengths 9 to 15 and a step size of one amino acid residue. All resultant peptides were compared to the reference database using the BLASTp command of the blast software package (e-value cut-off of 10, no gaps allowed). No significant alignments to human protein sequences could be found for peptide subsequences which were homologous to 100%.

Section 4: Safety Pharmacology

The ICH guideline S7A describes a core battery of studies including assessment of the function of the respiratory system, the central nervous system (CNS), and the cardiovascular system that should be performed on any pharmaceutical product prior to human exposure. Therefore safety pharmacology for RNA(LIP) was tested as integral part of six GLP toxicology studies that are described below.

Potential effects on the function of the CNS and respiratory system were evaluated in the pivotal repeated dose toxicity studies and did not reveal any test item-related influence on the animals.

A risk analysis on potential effects of RNA(LIP) vaccines was performed for the cardiovascular system. Systemically distributed RNA is degraded in the circulation and RNA formulated as RNA(LIP) is cleared from the blood within a few minutes and distributed mainly to the spleen and the liver as shown in biodistribution studies (see below). The obtained data do not suggest that RNA(LIP) will accumulate in the cardiovascular system. Potential systemic side effects of RNA(LIP) vaccination are expected to be associated with transient increase of IFN-α which is not expected to lead to cardiovascular side effects as documented in thousands of patients having received IFN-α. Consequently, a GLP cardiovascular safety pharmacology study compliant with ICH S7A/B was not performed. However, supportive ECG and blood pressure data from a non-GLP pharmacology study in cynomolgus monkey treated with RNA(LIP) are available and address assessment of cardiovascular function following treatment with RNA(LIP) vaccines.

In summary, no test item or treatment-related changes in the respiratory, neurological, and cardiovascular system were observed in any dose group tested in mice (respiratory system and CNS function) and cynomolgus (cardiovascular function).

Respiratory Safety

Respiratory safety was included in repeated dose toxicity studies in mice using RNA DPs coding for TAAs in compliance with GLP (LPT No. 28864 and 30283, for study design see below). For example, plethysmography was tested in the study with RNA(LIP) (LPT No. 30283) with four animals/sex/group treated either with control buffer, a low and a high dose (5 and 50 μg of RNA formulated with 9 μg and 90 μg of liposomes, respectively). A positive control of animals treated with 30 mg Carbamyl-β-methylcholine chloride (bethanecol)/kg b.w. was also included. Plethysmography was performed one day after the 4th to 7th dosing. The tests included the evaluation of respiratory rate, tidal volume, minute volume, inspiratory time, expiratory time, peak expiratory and inspiratory flow, expiratory time, and airway resistance index. None of tested pulmonary parameters showed any test item-related change in the treated animals, compared to the control group. Only animals of the positive control group showed expected alterations.

CNS Safety CNS safety was included in repeated dose toxicity studies in mice using RNA DPs coding for TAAs in compliance with GLP (LPT No. 28864 and 30283, for study design see below). For example, in the study with RNA(LIP) (LPT No. 30283) an observation screen was tested in five animals/sex approximately 24 h after the 5th dosing with control buffer, a low and a high dose (5 μg and 50 μg of RNA formulated with 9 μg and 90 μg of liposomes, respectively). Following tests were included in the observational screening: righting reflex, body temperature, salivation, startle response, respiration, mouth breathing, urination, convulsions, piloerection, diarrhea, pupil size, pupil response, lacrimation, impaired gait, stereotypy, toe pinch, tail pinch, wire maneuver, hind-leg splay, positional passivity, tremors, positive geotropism, limb rotation, and auditory function. In addition, functional tests to evaluate grip strength, and locomotor activity were included.

The neurological screening did not reveal any test item-related influence on the mice that would be attributed to a neurological toxicity. These findings were confirmed by the results of the GLP-compliant repeated dose toxicity study LPT No. 28864 conducted for the Lipo-MERIT study using different RNAs.

Cardiovascular Safety

A cardiovascular safety study according to ICH S7 was not done since RNA is degraded in the circulation within seconds and there is no indication that RNA(LIP) will accumulate in the cardiovascular system.

However, supportive data from a non-GLP pharmacology study in cynomolgus monkeys with RNA(LIP) targeting melanoma-associated antigens used in the Lipo-MERIT study are available. In this study, twelve cynomolgus monkeys were treated in six groups (see Table 11 for study design) and ECGs and blood pressure measurements were carried out after the 4^{th} dosing at three time points before dosing, 5 h after completion of the dosing, and 24 h after dosing. Treatment with RNA(LIP) was very well tolerated in cynomolgus monkeys (no observed clinical observation findings). None of the measured parameters (blood pressure, heart beat rate, QTc values, intervals of QT, P-segment, PQ, QRS) showed any test item related influence. In addition, serum levels of CK-MB and Troponin-I were measured to exclude the possibility of necrotic damage of heart muscle tissue. All the measured parameters were negative, supporting that there were no toxic effects of RNA(LIP) to the cardiovascular system at the dose levels tested in the study.

Discussion and Conclusions

Extensive studies were performed on the mode of action and the primary pharmacodynamics of RNA(LIP) in mice and in human in vitro test systems. The preclinical studies show that RNA(LIP) vaccines target the spleen and lymphoid tissue following IV administration. RNA(LIP) vaccine elicits dual effects, namely the induction of antigen-specific T-cell responses and cellular activation processes and immunomodulation following TLR triggering.

The data generated confirm that all antigen RNA lead structures applied in vivo induce antigen-specific T-cell responses, including the tetanus toxoid helper epitopes.

The functional properties of the RNA(LIP) formulation are (i) RNA protection in the serum and (ii) efficient in vivo targeting of APCs that are able to present antigenic peptides as well as getting activated following TLR7 triggering. The immunomodulatory activity of RNA led to dose-dependent cytokine induction in human samples, mice, and cynomolgus which all exhibited induction of IFN-α, IP-10, and IL-6 to various degrees, depending on the species tested or test system applied. RNA(LIP)-mediated cytokine induction in PBMCs was expected as there is good evidence from own RNA studies and literature. Apart from these expectations, the moderate induction of IFN-α and the induction of the chemokine IP-10 (CXCL10) reflects more likely the onset of the intended pharmacological effect than an unwanted immunotoxicological event.

Data generated in mice indicate that splenocytes are the main source of IFN-α secretion which is TLR7-dependent, as it diminished in TLR7−/− mice. We consider the observed transient and fully reversible cytokine responses as an intended pharmacodynamic effect contributing to the efficient induction of vaccine-induced anti-tumor T-cell responses. The favorable immunological properties were combined with a good tolerability of RNA(LIP) vaccines in mice and cynomolgus.

We also studied secondary effects of treatment with RNA(LIP) vaccines in several in vitro and in vivo studies using human, cynomolgus, and mouse test systems. A particular focus was set on the immunomodulatory effects of the RNA(LIP) vaccines as these were stronger than what we observed for non-formulated RNA vaccines administered into lymph nodes that only led to local cellular activation and cytokine induction.

Experiments using whole blood samples and PBMCs from human and cynomolgus donors were performed ruling out non-specific or uncontrolled cellular activation of human immune cells by RNA(LIP) vaccines yet showing moderate induction of cytokines as expected. In these experiments human cells and cynomolgus were treated at doses covering the highest intended clinical dose cohorts and above.

Although differences among donors, different in vitro test systems (cultured PBMCs vs. whole blood), or species were found in terms of cytokine levels, the observed cytokine patterns and the transient nature of cytokine responses were similar across all studies with only minor exceptions, such as IP-10 induction was not observed in cynomolgus. Human PBMCs showed induction of IP-10, and low response of IL-6, and IFN-α whose levels were even lower when examined in whole blood. Cynomolgus monkeys showed a very low IFN-α response, did not show any IP-10 induction and a more pronounced IL-6 response at the tested dose levels. Mice showed a strong response in IFN-α, IP-10, and IL-6, however at doses about 10-fold higher as tested in monkeys (based upon the doses per kg b.w.). The differences in cytokine expression between mice and monkeys might be explained by testing different doses on the one hand. On the other hand, mice have different activities for TLR7/8, which might also be a plausible explanation for different cytokine expression patterns.

The cytokine response patterns observed in cynomolgus in vivo were better reflected by the whole blood test system compared to the PBMC test system in which a broader, higher cytokine response at lower dose levels was observed. Still, the findings in the more sensitive PBMCs were integrated in the strategy to define a safe starting dose for patients. A side-by-side comparison of cytokine secretion in human and cynomolgus whole blood revealed that both species are highly comparable with regard to pro-inflammatory cytokine induction upon RNA(LIP) treatment suggesting that cynomolgus is an adequate animal model to predict secondary pharmacodynamic effects which may arise after vaccination with RNA(LIP) in patients.

In addition to cellular activation processes and cytokine induction following exposure to RNA(LIP) BioNTech assessed hematological changes in mouse and cynomolgus studies. Here, transient lymphopenia was observed equally in mouse and monkey at all dose levels. Overall, monkeys treated with RNA(LIP) show a similar reaction in cytokine profile and hematological parameters as observed for monkeys treated with other TLR agonist. This is in line with the assumption that the main activation processes of cytokine expression by RNA(LIP) occur via TLR stimulation. Extensive pharmacodynamics studies in wild-type, TLR7^{-/-} and IFNAR^{-/-} mice suggest that the hematological findings are secondary effects of RNA(LIP) induced cytokines. It has been shown that RNA(LIP) as well as non-formulated naked RNA is able to activate TLRs. TLR activation has been shown to induce lymphopenia and B-cell accumulation in the spleen. Supportively, histopathology data generated in the toxicological testing showed that transient lymphoid hyperplasia is found in the spleen but not in any other organ or tissue. This is in line with the observed lymphopenia in blood and emphasizes the intended targeting of RNA(LIP) and subsequently also the intended attraction of effector cells to the lymphoid organ. Safety pharmacology studies carried out suggest a safe profile for RNA(LIP). The neurological screening did not reveal any test item-related influence on the mice in any of the tests performed. None of tested pulmonary parameters showed any change in mice treated with RNA(LIP). In cynomolgus monkeys there were no indications for cardiovascular effects. Overall, RNA(LIP) exhibit a very good overall safety profile concerning safety pharmacology parameters.

Section 5: Pharmacokinetics

Even though pharmacokinetic studies are usually not performed during cancer vaccine development, we have undertaken in vivo studies to determine the biodistribution of intravenously injected RNA lipoplexes and the presence or persistence of residual plasmid amounts due to impurities in the drug product.

In vitro transcribed RNA consists of ribonucleotides and is hence identical in structure to RNA synthesized by the cells of the human body except for the 5′-cap structure. RNAs are therefore subject to the same degradation processes as natural mRNA. Especially in the extracellular space and serum, abundant RNases lead to rapid breakdown of RNA.

As outlined below, the distribution/disposition and potential accumulation of RNA in the spleen, liver, and lung were studied in pharmacokinetic studies. In addition, potential plasmid DNA impurities in gonads from mice treated with RNA(LIP) were quantified.

Biodistribution and persistence of the synthetic cationic lipid DOTMA has been investigated in first in vivo studies. Synthetic DOPE cannot be distinguished from the body's own natural phospholipid DOPE, should follow natural metabolization pathways and therefore biodistribution and accumulation was not further investigated.

Summary of Key Findings

Biodistribution of RNA(LIP), residual impurities of plasmid DNA, and the synthetic lipid DOTMA were analyzed in in vivo studies in mice.

RNA: For analysis of RNA(LIP) biodistribution in vivo, IVT-RNA levels in samples from a GLP-compliant repeated-dose toxicity study were analyzed by an RT-qPCR-based method at an external company. Organ samples for analysis of RNA included blood, spleen, liver, and lungs. A semi quantitative analytical method to detect the total amount of RNA was developed and organ samples were analyzed under non-GLP conditions. The method was based on a RT-qPCR method. RNA was rapidly cleared from blood with an estimated half-life of approx. 5 min. It was subsequently found in liver, spleen, and lung in much lower amounts as in blood.

Residual plasmid impurities: A quantitative analytical method to detect residual plasmid impurities was developed and gonad samples were analyzed in compliance with GLP at BioNTech IMFS GmbH. The method was based on a qPCR method to detect the kanamycin resistance gene on the plasmid.

Residual plasmid impurities were not detected or only slightly above the lower limit of detection. Signals were similar after the 1^{st} or 8^{th} dosing, suggesting that both RNA and residual DNA impurities do not accumulate or persist in the studied organs.

DOTMA: For analysis of RNA(LIP) biodistribution in vivo, DOTMA was extracted from blood and seven selected organs which were collected after IV RNA(LIP) injection into mice, and DOTMA content quantified by LC/MS analysis under non-GLP conditions. DOTMA was quickly (in less than one hour) delivered to the spleen (and other organs) after IV RNA(LIP) administration. The highest DOTMA findings were in the spleen and the liver, whereas DOTMA amounts in all other organ samples were rather negligible. Accumulated DOTMA after repetitive RNA(LIP) application is cleared from the organs with a kinetics which can be reasonably represented by a first order decay with an approximate half-life in the order of 6-7 weeks.

Biodistribution

RNA

The biodistribution of RNA(LIP) was studied in detail in mice by sampling organs during the GLP-repeated dose toxicity study (LPT No. 28864) performed for the clinical trial Lipo-MERIT. The organs were analyzed for the sum of all IVT-RNAs applying a quantitative real-time reverse transcription PCR (RT-qPCR) method developed at IMGM Laboratories GmbH, Martinsried, Germany, under non-GLP conditions (Study ID: RS297). In summary, the RNA was cleared very rapidly from blood with an estimated half-life of approximately 5 min. After 48 h and after seven days, RNA was detectable only at marginal level in blood and organs, suggesting that it is rapidly degraded and does not persist.

Residual Plasmid Impurities

The biodistribution of residual plasmid impurities from RNA(LIP) vaccination was investigated using samples from the GLP repeated-dose toxicity study (LPT No. 28864). A method was developed at BioNTech IMFS GmbH, Idar-Oberstein, Germany, in compliance with GLP to analyze the residual plasmid impurities in organ samples. All tested samples were either below or slightly above the lower limit of detection (LLOD), suggesting that plasmid DNA does not accumulate or persist in the gonads (Study ID: 36X130313).

DOTMA

The biodistribution of the two synthetic lipids used in RNA(LIP) formulations can provide insight into the physical distribution of the lipoplex carrier particle over time. The synthetic cationic lipid DOTMA was chosen for the biodistribution studies, because it is not a naturally occurring molecule, and can therefore be easily detected on the background of the biological matrix.

In an exploratory investigation of the DOTMA biodistribution, the lipid was extracted from blood and seven selected organs which were collected after IV injection of RNA(LIP) into mice. Here, a mixture of equal portions of liposome formulated IVT-RNAs of ATM quality was used. This initial study included five mice from which one was left untreated, two received a single injection of 60 μg RNA, and two received two injections of each 60 μg RNA in an interval of 20 days. All mice were sacrificed 24 h after the time point of the last injection. Quantification of DOTMA was performed by LC/MS measurements. Aim of the experiments was to test the general feasibility of the extraction and quantification protocols and to get a first hint on the biodistribution of DOTMA following RNA(LIP) vaccination.

DOTMA could be clearly determined from all investigated organs, and pronounced differences between the findings in different organs could be observed. In accordance with the proposed mode of action, highest DOTMA findings were in the spleen.

On the basis of these first results, a study with single administration of RNA(LIP) was performed (Report_BN_14_004). The DOTMA concentration in selected organs was assessed over a period of up to 28 days (d0, d1, d4, d7, d14, d21, and d28). In this experiment 200 μL of RNA(LIP), containing 20 μg of RNA and 26 μg DOTMA were administered (in the first study 60 μg were administered per injection). The DOTMA concentration in the administered product was 195 μM. Three mice per time point were investigated.

Evidently, DOTMA was predominantly found in spleen and liver with indication for slightly different accumulation kinetics. In all other investigated organs/tissues (lung, heart, kidneys, lymph nodes, fat pad, bone marrow, brain) the findings were by factors of 10 to 50 lower than that. From the data in liver and spleen, the pharmacokinetics of the DOTMA could be estimated: The maximum concentration was detected a few days after administration. Within 20 days the DOTMA concentration decreased to about 50% of the maximum values. These findings support the assumption that DOTMA is cleared from the organs within acceptable time scales, and no indication for a risk of permanent accumulation in any organ can be made out.

In a subsequent study, the concentration of DOTMA in selected organs was assessed prior (control group), during, and after eight weekly RNA(LIP), injections, each comprising 20 μg RNA (RBL005.2) and 26 ag DOTMA (Report_RB_15_004_V02). Organs were sampled from mice one hour after the first RNA(LIP) administration and then every other week after the preceding application. After completion of eight application cycles, mice were sacrificed after another 3, 6, 9, 12, and 15 weeks in order to investigate DOTMA clearance in the organs. Repetitive administration of RNA(LIP) test item and organ sampling was performed in-house whereas extraction and quantification of DOTMA from the provided organ samples was conducted by Charles River Laboratories Edinburgh Ltd. (Study No. 322915). The results were well in accordance with the previous studies conducted by us: Again, highest DOTMA concentrations were observed in the spleen as the main target organ followed by the liver. In all other organs, not more than about 5% of the concentration present in spleen samples was found (data not shown). DOTMA concentrations rose with increasing numbers of RNA(LIP) injections and then continuously decayed in the recovery phase after the last application. Terminal half-life of DOTMA in plasma (7.07 weeks), spleen (6.76 weeks), and liver (6.57 weeks) were comparable as obtained from the animals in the recovery period post 8^{th} dose.

In summary, DOTMA as an indicator for the lipid vehicle is quickly (in less than one hour) delivered to the spleen (and other organs) after IV RNA(LIP) administration. In addition to the spleen, DOTMA predominantly accumulates in the liver, where, however, no RNA translation is observed. In absolute numbers, the DOTMA amounts found in these two organs was close to the total cumulated DOTMA amount that was absolutely injected whereas DOTMA amounts in all other organ samples were rather negligible.

As assessed from animals in the recovery phase groups, accumulated DOTMA after repetitive RNA(LIP) application is cleared from the organs with a kinetics which can be reasonably represented by a first order decay with an approximate half-life in the order of 6 to 7 weeks. Such clearance kinetics is also in accordance with the findings from repetitive applications where transient accumulation was observed.

Taking together all results, these findings support the assumption that DOTMA is cleared from the organs within an acceptable time frame and that the potential risk of permanent lipid accumulation in plasma, liver, spleen, lung, heart, brain, kidney, uterus, lymph node, and bone marrow is rather low.

Discussion and Conclusions

IVT-RNA consisting of ribonucleotides has an identical structure as RNA produced by the cells of the human body with merely the 5′-cap as different structure. The IVT-RNAs are therefore subject to the same degradation processes as natural mRNA. Especially in the extracellular space and serum, abundant RNases lead to rapid breakdown of RNA.

The results of the RNA(LIP) biodistribution studies show high levels of RNA in blood shortly after the injection of RNA(LIP). RNA is rapidly cleared from blood and is found subsequently, albeit at much lower levels, in the spleen and liver whereas only marginal amounts could be found in lung. As RNA distributed to liver may lead to transient immune activation via TLR triggering, the liver enzymes will be closely monitored in patients following the first injection and throughout the study. After 48 h and seven days only residual amounts of RNA were found in blood and organs suggesting that RNA does not accumulate or persists in any organ. Also comparison of Cmax levels after the 1^{st} and 8^{th} injection did not show any accumulating effects.

In gonads, plasmid DNA was either not detected or samples were slightly above the LLOD, suggesting that there is only a minor risk of integration of plasmid residues, e.g., the Kanamycin resistance gene into the genome of germline cells.

Biodistribution of DOTMA has been shown to be primarily found in spleen and liver confirming spleen as the main target organ for RNA(LIP) vaccination and significantly lower exposed in plasma and other tissues after single and eight repetitive RNA(LIP) administrations. DOTMA was cleared from plasma, spleen, and liver with comparable terminal half-life in the order of 6 to 7 weeks. More systematic analyses concerning DOTMA biodistribution and accumulation will be carried prior to advanced clinical testing.

Section 6: Toxicology

The toxicology program for the RNA vaccine platform included several pharmacological studies to test RNA(LIP) vaccination across various dose ranges and repeated-dose toxicity studies including local tolerance and safety pharmacology parameters as well as immunotoxicity investigations. The studies were conducted in compliance with GLP conditions at an external CRO (LPT, Hamburg, Germany), using RNA and liposome batches comparable to the clinical trial material in terms of manufacturing processes and analytical quality controls. The GLP-compliant studies included a 6-week repeated dose toxicity study with IV administration of eight different RNA DPs encoding breast cancer antigens to C57BL/6 mice (LPT No. 30283).

In addition, a supplemental GLP-compliant 6-week repeated dose toxicity study was conducted employing the RNA vaccine platform with targeting a number of melanoma-specific antigens (LPT No. 28864). Although different RNA sequences were tested the toxicity data are also relevant for the application of the RNA DPs coding for TAAs and can add important information as the same type of liposomes was used for RNA(LIP) preparation. The toxicity profile of the formulated RNAs in both studies is supposed to be same or at least comparable due to the fact that possible side effects are related to the inherent molecular properties of liposome formulated RNAs, which are not dependent on RNA sequence and lengths.

Moreover, an additional 4-week repeated dose toxicity study was conducted to evaluate comparability of the liposomes used in the 6-week repeated dose toxicity study with a pH-adapted liposomal formulation of which buffer conditions were slightly adjusted due to long-term stability reasons (LPT No. 30586).

Summary of Key Findings

In the studies LPT No. 28864 and 30283, using the melanosomal and breast cancer antigen RNAs, no toxicological effects that could be attributed to the RNA(LIP) vaccination were observed.

No signs of local or systemic intolerance reactions were noted for the vaccinated animals. Body weight, food intake, drinking water consumption, functional observation tests, fore- and hind limb grip strength and spontaneous motility were not influenced by the test-items. Slight and transient lymphopenia was observed in animals of all dose groups which are considered to be in line with the intended pharmacological effect of RNA(LIP) vaccination due to TLR activation and cytokine induction. Most importantly, these effects were fully recovered after two weeks.

The chemokine IP-10 (CXCL10) and IFN-α were found to be transiently induced in a dose-dependent manner, likely due to the onset of the intended pharmacological effect. Induction of IP-10 and IFN-α was highest at 6 h after the 5^{th} injection and either close or completely back to normal levels after 24 h.

Transient substantial inductions of IL-6 and IFN-γ were found in male animals of the high dose groups only whereas only moderate inductions were observed for IL-2 irrespective of gender and dose level.

Test item-related increases of ALAT, ASAT, GLDH, and LDH were noted for animals of the high dose group, however, these effects were of transient nature and fully reversed after three weeks. No indications of liver toxicity were detected in histopathological examinations.

An increase in spleen weight was observed in animals of all dose groups. In animals of the mid and high dose group these effects were not fully recovered after three weeks. Urinary status and bone marrow were not influenced at any of the tested dose levels. No test item-related changes were noted at necropsy and at histopathological examination. A minimal to mild lymphoid hyperplasia of splenic white pulp due to the pharmacological mode of action was observed in animals vaccinated with the high dose. These effects were fully reversed after three weeks.

Importantly, as there were no findings in the low dose group of study LPT No. 30283 (5 μg total RNA) a NOAEL of 5 μg of total RNA per animal (i.e., approx. 0.2 mg/kg b.w. in mice) was reached for the RNA(LIP) vaccines.

In conclusion, the IV injection of multiple liposome formulated vaccine antigens was very well tolerated in mice. Comparing toxicity profiles of the liposomes used in the main studies and the slightly pH-adapted liposomal formulation no toxicologically noteworthy differences were observed between the two liposomal formulations (LPT No. 30586).

Selection of Relevant Species

We consider mice as the relevant species to test for potentially toxic direct effects of RNA(LIP) vaccines based on the following main reasons:

The mouse as model system provides all relevant features of innate and adaptive immunity relevant to characterize direct toxic effects of RNA DPs coding for TAAs. Mice exhibit all anticipated primary and secondary pharmacological effects from induction of CD4+/CD8+ T-cell responses to immunomodulatory effects that enhance the immunological response and lead to subsequent TLR triggering, cellular activation, and cytokine secretion.

The mouse system comprises an abundance of available tools and techniques for investigations of biological effects that outnumbers the experimental possibilities in other species by far (e.g., availability of transgenic mouse models, MHC tetramers, antibodies etc.). This enables more profound analysis of all unexpected events.

On-target effects of the vaccines cannot be investigated adequately in animal species. Thus, use of other animal species would not provide additional information and consequently use of higher mammals should not be considered.

Single-Dose Toxicology

Dose-range finding studies are usually performed to justify the doses for the pivotal toxicity study and to gain first information about target organs and signs of toxicity. We conducted several pharmacological studies to test RNA(LIP) across various dose ranges with schedules similar to the intended clinical regimen. During these studies the administration of RNA(LIP) was found to induce favorable pharmacodynamic effects and to be well tolerated.

In addition, previous toxicity studies showed that IV administered naked RNA is very well tolerated in mice also at high doses. Liposomes containing either DOTMA or DOPE as synthetic lipid components were tested in numerous clinical studies and several approved liposomal drug products proved a very good tolerability. Some liposomal formulations have been applied to reduce drug specific toxicities, e.g., nephrotoxicity or hepatotoxicity of nucleic acids at high doses or the toxicity of small molecules, such as doxorubicin or clofazimine.

The following was concluded from data generated by series of in-house studies and research of the literature:

Tolerable doses in mice that would provide a sufficient safety margin for the first dose in human use can be deduced from the performed pharmacology studies.

A single dose administration will not be sufficient to induce a significant immune response. A maximum immune response was observed after at least three applications.

RNA vaccines and lipoplex formulations are in general well tolerated.

Based on these conclusions we decided not to conduct single-dose toxicity studies but directly conducted a repeated-dose toxicity study.

Repeated-Dose Toxicology

The RNA(LIP) product of the RNA vaccine platform was analyzed for safety and toxicology in several GLP compliant repeated-dose toxicity studies addressing IV injection of RNA(LIP) products. Table 13 provides an overview of the GLP repeated-dose toxicity studies that support that support the clinical phase 1 and 2 testing using RNA(LIP) vaccines.

TABLE 13
Design of GLP repeated dose toxicity studies.

| Study | Study Design |
|---|---|
| 6-week | In total eight IV administrations into the tail |
| repeated- | vein on day 1, 4, 8, 11, 15, 22, 29, and 43, |
| dose toxicity | followed by a 3-week recovery period |
| study of | Vaccine: RBL001.1, RBL002.2, RBL003.1, |
| RNA(LIP) | and RBL004.1 RNA(LIP) 4 groups |
| by IV admin- | (14 animals/sex/group): |
| istration to | 1. control: vehicle |
| C57BL/6 | 2. low dose: 4 × 3.75 μg (total RNA amount: |
| mice (LPTNo. | 15 μg; total amount DOTMA 17.4 μg, total |
| 28864) | amount DOPE: 9.3 μg) |
| | 3. mid dose: 4 × 7.5 μg (total RNA amount: |
| | 30 μg; total amount DOTMA 34.8 μg; total |
| | amount DOPE: 18.6 μg) |
| | 4. high dose: 4 × 15 μg (total RNA amount: |
| | 60 μg; total amount DOTMA: 69.6 μg; total |
| | amount DOPE: 37.2 μg) |
| 6-week | In total eight IV administrations into the tail |
| repeated- | vein of on day 1, 4, 8, 11, 15, 22, 29, and 43, |
| dose toxicity | followed by a 3-week recovery period |
| study of | Vaccine pool 1: |
| RNA(LIP) | RBL008.1, RBL005.2, RBL006.2, RBL007.1, |
| co-formulated | co-formulated with RBLTet.1 (tetanus toxoid |
| with RBLTet.1 | helper epitope P2P16). |
| by IV admin- | Vaccine pool 2: |
| istration to | RBL008.1, RBL009.1, RBL010.1, RBL011.1, |
| C57BL/6 | co-formulated with RBLTet.1 (tetanus toxoid |
| mice (LPT | helper epitope P2P16). |
| No. 30283) | 5 groups (14 animals/sex/group): |
| | 1. control: vehicle |
| | 2. low dose: Vaccine pool 1 (0.6 μg RBLTet.1 + |
| | 4 × 1.1 μg Antigen RNA; total amount |
| | DOTMA: 5.8 μg; total amount DOPE: 3.1 μg) |
| | 3. high dose: Vaccine pool 1 (6 μg RBLTet.1 + |
| | 4 × 11 μg Antigen RNA; total amount DOTMA: |
| | 58.0 μg; total amount DOPE: 31.0 μg) |
| | 4. low dose: Vaccine pool 2 (0.6 μg RBLTet.1 + |
| | 4 × 1.1 μg Antigen RNA; total amount DOTMA: |
| | 5.8 μg; total amount DOPE: 3.1 μg) |
| | 5. high dose: Vaccine pool 2 (6 μg RBLTet.1 + |
| | 4 × 11 μg Antigen RNA; total amount |
| | DOTMA: 58.0 μg; total amount DOPE: 31.0 μg) |
| 4-week | In total five IV administrations into the tail vein |
| repeated | of on day 1, 4, 8, 11, and 25, followed by a 2- |
| dose toxicity | week recovery period |
| study of two | Vaccine: RBL008.1, L1 Liposomes, L2 |
| liposomal | Liposomes |
| RBL008.1 | 2 groups (9 animals/sex/group): |
| formulations | 1: 20 μg RBL008.1 + 40 μg L1 |
| by IV admin- | Liposomes/animal |
| istration to | 2: 20 μg RBL008.1 + 40 μg L2 |
| C57BL/6 | Liposomes/animal |
| mice (LPT | |
| No. 30586) | |

ATM Formulation

The composition, formulation, and specifications of the AMT were planned as close as possible to the intended drug product for use in humans.

Minor changes in the RNA(LIP) preparation process had to be made owing to following reasons:

To obtain a high dose that increases the likelihood to capture potential dose-dependent toxicological effects and thereby fulfills criteria for toxicity testing as outlined in guidelines ICH S6 or M3(R2).

To prevent administration above the feasible maximum volume in mice which is 250 μL volume in a slow bolus injection. Higher injection volumes were ethically not recommended and were bearing the risk of losing mice during the injection.

Differences to the Clinical Protocol are:

Patients will obtain the different RNA(LIP) products in a consecutive manner. This was not possible in mice because of the limitation to the volume. RNA(LIP) for mice were prepared individually, then mixed, and all four RNA lipoplexes were injected at the same time in a total volume of 250 μL.

For the formulation of RNA(LIP) for treatment of patients 150 mM NaCl will be used. In order to obtain higher doses in the toxicity studies, a NaCl solutions with higher concentrations had to be used for the RNA(LIP) formation.

For the preparation of RNA(LIP) for patient treatment RNA drug products with a concentration of 0.25 mg/mL will be used. In order to obtain the high dose in the toxicity study, higher concentrated RNAs, i.e., 1 mg/mL, had to be used for preparation of RNA(LIP) products in study LPT No. 28864.

In this trial acetic acid stabilized liposomes (L4) which are half concentrated with respect to previously used, equivalently manufactured acetic acid stabilized liposomes (L2), will be applied. No further bridging studies were planned as the same characteristics as for L2 Liposomes are given.

It is our position that the mentioned changes to the formulation protocol in ATM have no or little influence on the results or conduct of the studies.

Study Design

For the study design see Table 13. According to ICH S6 and S8 the data from the standard toxicity studies were evaluated for signs of immunotoxic potential. The following investigations were performed in accordance with FDA, ICH, and CHMP guidance documents: mortality, histopathology (especially spleen), gross pathology, and organ weight, clinical observations, ophthalmology, local tolerance, injection site reactions, body weight, food consumption, standard hematology parameters, and clinical chemistry and cytokines (IL-1β, IL-2, IL-6, IL-10, IL-12, TNF-α, INF-α, INF-γ, and IP-10).

Safety pharmacology studies were included to test for the respiratory and central nervous system, as outlined in Section 4.

Results

The toxicological assessment in the 6-week repeated-dose toxicity studies using the vaccine platform revealed only minor effects that can primarily be attributed to the anticipated pharmacological mode of action of RNA(LIP) (Table 14). Desired immunomodulatory effects of RNA(LIP) are TLR activation and release of cytokines. The induction of IFN-α in mice leads to secondary effects like leukopenia, decrease of platelets, and increase of liver parameter such as ALAT, effects that are commonly described for patients treated with IFN-α. In line with this, test item-related changes in treated animals were mainly transient (Table 14). In addition, transient activation of cytokines IP-10, IFN-α, IL-6, and IFN-γ was observed. All inductions were back to normal levels after 24 h (apart from IP-10 levels which were still slightly above normal levels).

The hematological findings in mice included mainly lymphocytopenia and low, reversible decreases in total leucocytes, neutrophils, reticulocytes, and thrombocytopenia in all treatment groups. These findings were fully reversible. Lymphoid hyperplasia observed for the spleen in the histopathology was fully recovered and outlines a desired effect and the intended targeting of the test substance and lymphocytes to the spleen.

Observed mild changes in liver parameters such as GLDH, LDH, ALAT, and ASAT affected mainly the high dose groups and were not noticed in animals of the recovery group, suggesting a full recovery of the effects within at least three weeks or less. There were no liver toxicities detected in histopathology.

As there were no findings in the low dose group in the study LPT No. 30283 the NOAEL was met at a dose of 5 μg of total RNA per animal (i.e., approx. 0.2 mg/kg b.w. in mice). A further 4-week repeated-dose toxicity study was performed to address a change in the composition of the liposome buffer (LPT No. 30586). The data prove that the new type of liposomes is absolutely comparable in the measured parameters to the one used in the main study.

TABLE 14
Overview of toxicological findings in the repeated-dose
toxicity studies using RNA(LIP) (LPT No. 28864,
30283, and 30586).
All the described findings were statistically significant
in comparison with the control group.

| | Study | |
|---|---|---|
| Category | No. | Findings |
| Mortality | LPT No. | One animal of the high dose group died |
| | 28864 | prematurely immediately after the 5th |
| | | injection. The animal revealed an enlarged |
| | | spleen and moderate extra-medullary |
| | | hematopoiesis. The death was considered |
| | | related to the administration of the test |
| | | item. |
| | LPT No. | No premature deaths occurred during the |
| | 30283 | study. |
| | LPT No. | One animal of the L2 Liposome group |
| | 30586 | died prematurely 4 days after the 4th |
| | | injection. No premortal symptoms were |
| | | noted. The animal revealed an enlarged |
| | | ovary and autolysis of all organs. The |
| | | death was considered to be of |
| | | spontaneous nature. |
| Body | LPT No. | Food consumption, body weight, and |
| weight, | 28864 | body weight gain were |
| food | LPT No. | not influenced in all studies. |
| con- | 30283 | |
| sumption | LPT No. | |
| | 30586 | |
| Local | LPT No. | Signs of local intolerance were not noted in |
| tolerance | 28864 | any of the studies. |
| | LPT No. | |
| | 30283 | |
| | LPT No. | |
| | 30586 | |
| Clinical | LPT No. | Slightly reduced motility was observed in |
| signs | 28864 | nine animals of the high dose group after |
| | | the 5th injection, starting at 5 min after |
| | | injection, lasting for approx. 15 min. |
| | | Thereafter, the animals showed normal |
| | | behavior. This was observed again |
| | | in one animal after the 6th and 7th injection. |
| | | In one female, slight ataxia was |
| | | observed at the same time. |
| | | However, although noticed only in the high- |
| | | dose group, these findings are considered |
| | | to be of spontaneous nature due to |
| | | the single occurrence, probably resulting |
| | | from the animal handling during the |
| | | administration procedure. This assumption |
| | | is supported by the fact that no biologically |
| | | relevant changes were measured during |
| | | locomotor activity assessment within the |
| | | scope of neurological screening. |
| | LPT No. | No signs of systemic intolerance were noted |
| | 30283 | for any animal. |
| | LPT No. | No signs of systemic intolerance were noted |
| | 30586 | for any animal. |
| Neuro- | LPT No. | Was started 24 h after the 5th dosing. No test |
| logical | 28864 | item-related influence was noted in any animal. |
| screening | LPT No. | Was started 24 h after the 5th dosing. No test |
| | 30283 | item-related influence was noted in any animal. |
| | LPT No. | Not done. |
| | 30586 | |
| | LPT No. | Was started 24 h after the 8th dosing. No test |
| | 28864 | item-related influence was noted. |
| Plethys- | LPT No. | Was started 24 h after the 4th dosing. No test |
| mography | 30283 | item-related influence was noted. |
| | LPT No. | Not done. |
| | 30586 | |
| Hema- | LPT No. | A decrease of white blood cell counts, |
| tology | 28864 | lymphocytes, an platelets and an increase of |
| and | LPT No. | neutrophils and large unstained cells were |
| coagu- | 30283 | observed in all dose groups. The effects were |
| lation | | fully recovered and are considered in line |
| | | with the pharmacological effect of |
| | | RNA(LIP) due to TLR activation andI FN-α |
| | | induction (see also Section 0). |
| | LPT No. | No test item-related changes between both |
| | 30586 | groups were noted in hematological |
| | | parameters. |
| Clinical | LPT No. | Some changes compared to control animals |
| bio- | 28864 | were found in liver enzymes as summarized |
| chemistry | LPT No. | below. There was no indication of liver |
| | 30283 | toxicity in the histopathological examinations |
| | | (samples for clinical biochemistry were taken |
| | | shortly before necropsy). |
| | | Cholesterol levels were increased in all dose |
| | | groups. Alanine aminotransferase (ALAT), |
| | | lactate dehydrogenase (LDH), and |
| | | glutamate-dehydrogenase (GLDH) levels |
| | | were significantly increased mainly in the |
| | | high dose group (60 μg/animal). Cholesterol |
| | | levels were increased in all dose groups. |
| | | ALAT, |
| | | Aspartate amino-transferase (ASAT), LDH |
| | | and GLDH levels were significantly increased |
| | | in the high dose groups (50 μg/animal), more |
| | | pronounced in male animals. |
| | LPT No. | No test item-related changes between both |
| | 30586 | groups were noted in clinical biochemistry |
| | | parameters. |
| Urine | LPT No. | Urine parameters were not influenced in any |
| analysis | 28864 | of the studies. |
| | LPT No. | |
| | 30283 | |
| | LPT No. | |
| | 30586 | |
| Ophthal- | LPT No. | Was not influenced in any of the studies. |
| mology | 28864 | |
| and | LPT No. | |
| auditory | 30283 | |
| system | LPT No. | |
| | 30586 | |
| Cytokines | LPT No. | Observations in dose groups 2, 3, and 4: IP- |
| | 28864 | 10: dose-dependent induction with peaks |
| | | 6 h after the 4th administration (up to 29- |
| | | fold induction). Levels were close normal |
| | | levels after 24 h (4- to 5-fold over control |
| | | group). |
| | | IL-6: dose-dependent induction with peaks |
| | | at 6 h after the 4th administration (up to 8- |
| | | fold induction). Levels were normal after |
| | | 24 h. |
| | | IL-10: induction with peaks at 6 h after the |
| | | 4th administration (up to 3.5-fold induction) |
| | | in females only. |
| | | Levels were normal after 24 h. |
| | | TNF-α: low induction with peaks at 6 h after |
| | | the 4th administration (up to 2.5-fold |
| | | induction). Levels were close normal levels |
| | | after 24 h (2-fold in females of the high dose |
| | | group). |
| | | Observations only in dose group 4 (high dose): |
| | | IFN-γ: Induction in males (518%) and females |
| | | (88%) 6 h after the 4th dosing returned to |
| | | normal levels after 24 h. |
| | LPT No. | Observations in all dose groups: |
| | 30283 | IP-10: dose-dependent induction with peaks |
| | | 6 h after the 5th administration (up to 41- |
| | | fold induction). Levels were close normal |
| | | levels after 24 h (4- to 5-fold over control |
| | | group). |
| | | Observations in high dose groups (groups 3 |
| | | and 5): |
| | | IL-6: induction with peaks at 6 h after the 5th |
| | | administration (up to 20-fold induction). |
| | | Levels were normal after 24 h. |
| | | IFN-α: induction with peaks at 6 h after the |
| | | 5th administration (up to 55-fold induction). |
| | | Levels were normal levels after 24 h. |
| | | IFN-γ: Induction in group 3 (males 176-fold) |
| | | and group 5 (males 49-fold) 6 h after the 5th |
| | | dosing, returned to normal levels after 24 h. |
| | LPT No. | No test item-related changes between both |
| | 30586 | groups were noted in cytokine measurement. |
| Comple- | LPT No. | Slight increase of C5a 24 h after the 6th |
| ment | 28864 | dosing in group 3 (13/70% in male/female) |
| | | and dose group 4 (42/90%). |
| | LPT No. | Slight increase (up to 71%) of C5a 24 h after |
| | 30283 | the 5th dosing in all dose groups, in female |
| | | animals only. The levels returned to normal |
| | | after 48 h. |
| | LPT No. | Not determined. |
| | 30586 | |
| Macro- | LPT No. | No test item-related macroscopic systemic |
| scopic | 28864 | changes were noted for all dose groups |
| post | LPT No. | during the treatment or recovery period |
| mortem | 30283 | in all studies. |
| findings | LPT No. | |
| | 30586 | |
| Organ | LPT No. | A slight increase in liver weight (19%) was |
| weights | 28864 | observed in male animals of all dose groups, |
| | | and in female animals of group 3 (13%). The |
| | | effects were fully recovered after 3 weeks. |
| | | There was no indication of liver toxicity in |
| | | the histopathological examinations. This |
| | | finding is of no biological relevance because |
| | | liver weights were within the normal range |
| | | of biological variations. |
| | | An increase in spleen weight (ranging from |
| | | 61-107%) was observed in animals of all |
| | | dose groups. The effects were not fully |
| | | recovered after 3 weeks in animals of the |
| | | mid and high dose groups. |
| | LPT No. | An increase in spleen weight was dose |
| | 30283 | dependent and observed in animals of all |
| | | dose groups (ranging from 33-53% in the |
| | | low dose group and 72-85% in the high dose |
| | | group). The effects were not fully recovered |
| | | after 3 weeks (ranging from 19-22% in the |
| | | low dose group and 40-74% in |
| | | the high dose group). |
| | LPT No. | No test item-related changes between both |
| | 30586 | groups were noted in organ weights. |
| Bone | LPT No. | Myeloid: erythroid ratios were not |
| marrow | 28864 | influenced in any of the studies. |
| | LPT No. | |
| | 30283 | |
| | LPT No. | |
| | 30586 | |
| Histo- | LPT No. | Examination was restricted to control and |
| path- | 28864 | groups 3 and 4. |
| ology | | Spleen: lymphoid hyperplasia of the white |
| | | pulp in animals of group 3 and 4. |
| | | Thymus: marked atrophy in female animals |
| | | only in animals of group 3 and 4. |
| | | All effects were fully recovered after 3 |
| | | weeks. |
| | | Lymphoid hyperplasia was not observed in |
| | | any other organ. The observed effects in |
| | | lymphoid organs are due to the pharma- |
| | | cological mode of action and not considered |
| | | an adverse reaction. |
| | LPT No. | Examination was restricted to control and |
| | 30283 | group 3 and 5. Spleen: lymphoid |
| | | hyperplasia of the white pulp in animals of |
| | | the high dose groups. All effects were fully |
| | | recovered after 3 weeks. |
| | | Lymphoid hyperplasia was not observed in |
| | | any other organ. The observed effects in |
| | | lymphoid organs are due to the pharma- |
| | | cological mode of action and not considered |
| | | as an adverse reaction. |
| | LPT No. | The histopathological examination was |
| | 30586 | restricted to the main study animals treated |
| | | with 20 μg RBL008.1/animal +40 μg L2 |
| | | Liposomes/animal (group 2). No morpho- |
| | | logical lesions were noted that are |
| | | considered to be related to the administration |
| | | of RBL008.1 combined with L2 Liposomes. |
| | | All observations were considered to be |
| | | within the normal range of background |
| | | alterations, which may be seen in untreated |
| | | mice of this age and strain. |

Genotoxicity

The components of RNA(LIP) products (lipids and RNA) are not suspected to have genotoxic potential. No impurity or component of the delivery system warrants genotoxicity testing. In accordance with recommendations given in the ICH guideline on Preclinical safety evaluation of biotechnology-derived pharmaceuticals S6(R1) (June 2011) no genotoxicity studies are planned.

Carcinogenicity

RNA itself and lipids used as vehicles have no carcinogenic or tumorigenic potential. In accordance with ICH S1A, no long-term carcinogenicity studies are required in cases where there is no cause for concern derived from laboratory and toxicology studies and where no chronic application of the drug is intended expectancy.

Reproductive and Developmental Toxicity

Macroscopic and microscopic evaluations of male and female reproductive tissues were included in the repeated-dose toxicity studies in mice treated with RNA(LIP). No findings were noted in these studies, thus no specific fertility and developmental toxicity studies will be performed prior to initiation of the phase 1 studies with RNA(LIP) vaccines. Direct cytotoxic effects on reproductive tissues are not expected with RNA(LIP) as supported by the experience from other cancer vaccines showing no effects on reproduction and development. Since effects on reproduction cannot be excluded, women of childbearing potential will have to use effective contraception during treatment. No further long-term or reproductive toxicity studies are planned at this point.

Local Tolerance

According to ICH recommendation, testing for local tolerance was evaluated in the GLP repeated dose toxicity study for IV injection. Signs of local intolerance were not observed during the studies.

Other Toxicity Studies

Antigenicity

Due to the fast extracellular breakdown of IVT-RNA within seconds to minutes no formation of anti-drug antibody (ADA) is expected. Therefore no specific antigenicity testing regarding antibody induction is planned.

Immunotoxicity

Since it is the intention to activate the immune system by the RNA(LIP) products, particular attention was paid on immunotoxicological parameters to exclude unintended activation or suppression. Inspection of immunotoxicology was implemented in both the 6-week repeated-dose toxicity studies (LPT No. 28864 and 30283). In addition to monitoring cytokine levels in the serum, the following relevant parameters were considered to evaluate immunotoxicity: body weight, body temperature, weight of lymphatic organs, macroscopic and histopathology of lymphatic organs, absolute and relative differential blood count, total serum protein, albumin/immunoglobulin ratio, myeloid/erythroid ratio in the bone marrow, coagulation parameters.

Hematology

A decrease in lymphocytes, white blood cell counts (mainly due to the lymphocyte decrease) and platelets was observed in all treatment groups on test day 44, approximately 24 h after the 8th injection in both studies. All effects were fully recovered after two weeks. The results are shown in Table 15 and Table 16 for studies LPT No. 28864 and 30283, respectively.

TABLE 15
Hematology data (LPT No. 28864).
Samples for hematology determination were taken on test day 44
(approx. 24 h after the 8th injection).
Changes in hematological parameters compared to the control group
(mean values) at the end of the treatment period (test day 44) [%]

| | | Group 2 | | Group 3 | | | |
|---|---|---|---|---|---|---|---|
| | | 15 μg/animal | | 30 μg/animal | | Group 4 60 μg/animal | |
| Parameter | | males | females | males | females | males | females |
| Leukocytes (WBC) | | −72 ** | −60 ** | −78 ** | −61 ** | −68 ** | −69 ** |
| Neutrophilic | -rel. | +100 | +50 | +163 | +58 | +74 | +36 |
| granulocytes (Neut) | -abs. | −46 * | −30 | −44 * | −29 | −47 * | −51 |
| Lymphocytes (Lym) | -rel. | −16 | −10 | −19 | none | −10 | none |
| | -abs. | −77 ** | −64 ** | −82 ** | −65 ** | −71 ** | −71 ** |
| Monocytes (Mono) | -rel. | +89 | −21 | +68 | none | −24 | −29 |
| | -abs. | −44 ** | −64 | −59 ** | −43 | −72 ** | −79 * |
| Eosinophilic | -rel. | +41 | +68 | +90 | +45 | −34 | −23 |
| granulocytes (Eos) | -abs. | −59 ** | −43 | −59 ** | −38 | −78 ** | −73 * |
| Large unstained cells | -rel. | +385 | +315 | +257 | +239 | +259 | +275 |
| (LUC) | -abs. | +59 | +28 | −25 | none | +13 | none |
| Basophilic | -rel. | +255 | +144 | +445 | +50 | +273 | +80 |
| granulocytes (Baso) | | | | | | | |
| Platelets (PCT) | | −35 ** | −38 ** | −32 ** | −39 ** | −46 ** | −39 ** |

* Statistically significant, p ≤0.05;
** statistically significant, p ≤0.01 (Dunnett's test).

TABLE 16
Hematology data (LPT No. 30283).
Samples for hematology determination were taken on test day 44
(approx. 24 h after the 8th injection).
Changes in hematological parameters compared to the control group
(mean values) at theend of the treatment period (test day 44) [%]

| | | Group 2: | | Group 3: | | Group 4: | | Group 5: | |
|---|---|---|---|---|---|---|---|---|---|
| | | 5 μg/animal | | 50 μg/animal | | 5 μg/animal | | 50 μg/animal | |
| | | (RNAset 1) + | | (RNAset 1) + | | (RNAset 2) + | | (RNAset 2) + | |
| | | 9 μg/animal | | 90 μg/animal | | 9 μg/animal | | 90 μg/animal | |
| | | (liposomes) | | (liposomes) | | (liposomes) | | (liposomes) | |
| Parameter | | males | females | males | females | males | females | males | females |
| Leukocytes | | −15 | 54 | −65 | −39 | −51** | −48 | −71 ** | −57 ** |
| (WBC) | | | | | | | | | |
| Platelets (PCT) | | −18 * | none | −48 ** | −43 ** | −16 ** | none | −45 ** | −46 ** |
| Reticulocytes | | None | −30 ** | −27 ** | −34 ** | −17 ** | −25 ** | −27 ** | −38 ** |
| (Ret) | | | | | | | | | |
| Neutrophilic | -rel. | +125 | +24 | +199 | +32 | +81 | +22 | +234 | +72 |
| granulocytes | | | | | | | | | |
| (Neut) | | | | | | | | | |
| Lymphocytes | -rel. | −11 | −5 | −21 | −8 | −11 | −4 | −20 | −9 |
| (Lym) | -abs. | −21 | −56 * | −73 | −43 | −56 *− | 49 * | −89 ** | −61 * |
| Monocytes | -rel. | None | none | −12 | −74 | none | none | −41 | −47 |
| (Mono) | -abs. | None | none | −74 * | −93 * | none | none | −76 ** | −71 |
| Large | -rel. | +86 | +29 | +516 | +640 | +194 | +195 | +320 | +295 |
| unstained | -abs. | None | none | +80 * | +336 * | +30 | +50 | none | +71 * |
| cells (LUC) | | | | | | | | | |
| Basophilic | -rel. | None | none | +540 | +290 | none | none | +300 | +90 |
| granulocytes | | −18 * | none | −48 ** | −43 ** | −16** | none | −45 ** | −46 ** |
| (Baso) | | | | | | | | | |
| Platelets (PCT) | | | | | | | | | |

* Statistically significant, p ≤0.05;
** statistically significant, p ≤0.01 (Dunnett's test).

Cytokine Determination

Excretion of following cytokines was analyzed in the repeated-dose toxicity studies: IL-1β, IL-2, IL-6, IL-10, IL-12p70, TNF-α, IFN-γ, and IP-10 that are known to be sensitive indicators of immune activation or TLR7 signaling. In the toxicity study LPT No. 28864 mice revealed a dose-dependent and test item-related increase of the cytokine IP-10. IP-10 levels increased transiently and were highest at 6 h after the 4th injection. After 24 h, the levels were still significantly higher compared to control levels, but were already nearly back to normal levels. Compared to the control group, IP-10 showed a maximum induction of 28- and 16-fold (in males and females, respectively) after 6 h. A significant increase was also observed for TNF-α (only females, group 2, 3, and 4), IL-10 (only females, group 3 and 4), IL-6 (male, group 4), and IFN-γ (male, group 4). The maximum inductions were 3-fold for TNF-α, 4-fold for IL-10, 7- and 8-fold for IL-6, and 6- and 2-fold for IFN-γ. All effects were fully reversible after 24 h (with the exception of TNF-α levels in females of group 4). The results are summarized in Table 17.

TABLE 17
Cytokine levels in plasma (LPT No. 28864).
Samples for cytokine determination were taken 6 h and 24 h after the 4^{th} injection.
Test item-related changes in cytokine levels (mean values), expressed as
x-fold increase over the control group level if applicable

| | | Group 2 15 μg/animal | | Group3 30 μg/animal | | Group 460 μg/animal | |
|---|---|---|---|---|---|---|---|
| Cytokine | Time | males | females | males | females | males | females |
| IL-6 | 6 h | 2x | 2x | 3x | 7x ** | 7x ** | 8x ** |
| IL-10 | 6 h | none | 2x | none | 4x ** | none | 4x ** |
| | 6 h | 17x ** | 11x ** | 22x** | 17x ** | 29x ** | 17x ** |
| IP-10 | 24 h | 4x ** | 4x ** | 4x ** | 4x ** | 5x ** | 5x ** |
| IFN-γ | 6 h | none | none | none | none | 6x ** | 2x |
| TNF-α | 6 h | 1x | 2x * | 1x | 2x ** | 1x | 2x ** |
| | 24 h | none | none | none | 1x | none | 2x ** |

* Statistically significant, p ≤0.05;
** statistically significant, p ≤0.01 (Dunnett's test).

For cytokine determination in study LPT No. 30283 serum samples were taken 6 h and 24 h after the 5th injection. Increased serum levels of IL-2, IL-6, IP-10, IFN-α, and IFN-γ were found (Table 18). A clearly dose-dependent induction was noted for IL-6. For IFN-γ, an induction was noted after high-dose treatment (statistically significant at p≤0.01), being more pronounced in the male animals. For IFN-α, an induction was observed after low dose and after high dose treatment (statistically significant at p≤0.01 or p≤0.05), being more pronounced for the female animals in group 5 (RNA Set 2, high dose). The induction of all abovementioned cytokines had subsided at 24 h post administration.

A relatively low, but dose-related induction for IL-2 was noted for the male and female animals 6 h and 24 h after low or high-dose treatment.

A pronounced dose-dependent effect was detected for IP-10 at all dose levels for the male and female animals compared to the control group at 6 h after high-dose treatment. At 24 h post administration, the IP-10 levels were still increased compared to the control group at all dose levels. This induction of IP-10 reflects an intended pharmacological effect and is not regarded as an unwanted immunotoxicological event.

TABLE 18
Cytokine levels in plasma (LPT No. 30283).
Samples for cytokine determination were taken 6 h and 24 h after the 5^{th} injection.
Test item-related changes in cytokine levels (mean values), expressed as x-fold
increase over the control group level if applicable , or as increase only

| | | Group 2: | | Group 3: | | Group 4: | | Group 5: | |
|---|---|---|---|---|---|---|---|---|---|
| | | 5 μg/animal | | 50 μg/animal | | 5 μg/animal | | 50 μg/animal | |
| | | (RNAset 1) + | | (RNAset 1) + | | (RNAset 2) + | | (RNAset 2) + | |
| | | 9 μg/animal | | 90 μg/animal | | 9 μg/animal | | 90 μg/animal | |
| | | (liposomes) | | (liposomes) | | (liposomes) | | (liposomes) | |
| Cytokine | Time | Males | females | males | females | males | females | males | females |
| IL-2 | 6 h | incr. | 1x | incr. | 2x | none | 1x | incr. ** | 31x |
| | 24 h | none | incr. | incr. * | incr. | incr. | incr. | incr. | incr. |
| IL-6 | 6 h | 2x | incr. | 22x ** | incr.** | 2x | incr. | 21x ** | incr.** |
| | 24 h | none | none | none | 2x | 1x | 3x | 1x | 2x |
| IP-10 | 6 h | 18x * | 11x ** | 41x ** | 25x ** | 20x ** | 14x ** | 34x ** | 22x ** |
| | 24 h | 4x * | 3x ** | 6x** | 5x ** | 3x ** | 3x * | 4x ** | 4x ** |
| IFN-α | 6 h | 5x * | 8x * | 15x ** | 28x ** | 8x * | 9x | 13x ** | 55x ** |
| | 24 h | none | none | none | none | none | none | none | none |
| IFN-γ | 6 h | 4x | incr. | 176x ** | incr. ** | 2x | incr. | 49x** | incr.** |
| | 24 h | none | none | none | none | none | none | none | none |

incr.: a clear increase was noted compared to control group, however as the control group value was set to ‘0.0’ the increase cannot be expressed as a multiple.
* Statistically significant, p ≤0.05;
** statistically significant, p ≤0.01 (Dunnett's test).

A cytokine determination was also performed in the course of the study comparing L1 and L2 Liposomes (LPT No. 30586). Here the cytokines were analyzed 6 h and 24 h after the 4th immunization. No test-item related changes between the groups were noted.

Discussion and Conclusion

Treatment with RNA(LIP) is well tolerated in mice, as shown for a number of antigen-encoding RNAs assessed in three different repeated-dose toxicity studies (LPT No. 28864, 30283, and 30586). Overall, the treatment with up to eight IV injections was well tolerated, also in animals of the high dose groups. No test item-related premature deaths were observed in the studies No. 30283 and 30586. One animal died prematurely after administration of the test item in study No. 28864. As there were no findings in the low dose group of study No. 30283 the NOAEL was reached at a dose of 5 μg of total RNA per animal (i.e., approx. 0.2 mg/kg b.w. in mice). In addition, vaccination with RNA(LIP) products was also very well tolerated in a non-GLP pharmacology study in twelve cynomolgus monkeys (no clinical observation findings). The toxicological assessment of RNA(LIP) in the repeated-dose toxicity studies in mice revealed effects including transient induction of cytokines, hematological changes, and elevation of liver enzymes that could be attributed to the test item. Observed effects were mainly the induction of the cytokines IP-10, IFN-α, IFN-γ, and IL-6 in the in vivo studies reported here, as well as in the in vitro studies described and discussed above.

Notably, none of the pro-inflammatory cytokines such as TNF-α, IFN-γ, or IL-2 were up-regulated in an excessive manner in mice. However, in the cynomolgus study, at least one animal revealed a high transient induction of IL-6 (1,076 μg/mL). IL-6 induction was also observed in mice in a dose-dependent manner, but to a lower extent. IL-6 along with other cytokines will be monitored carefully throughout the clinical study and directly analyzed in patients.

Effects such as lymphopenia and liver enzymes elevation were also reported after treatment with plasmid lipoplexes and by TLR activation in mice and monkeys and are commonly observed as secondary effects driven by IFN-α secretion that has been commonly described for patients treated with recombinant IFN-α which is on the market for many years for the treatment of several oncological and non-oncological diseases.

Observed changes in liver parameters of the high dose group in mice suggest that the liver may be a target of toxicity at higher doses of liposome formulated RNA. The changes include increase in liver weight, increase in GLDH-, LDH-, ASAT-, and ALAT-levels in plasma. These changes are regarded as mild and were not observed in animals of the recovery group, suggesting a full recovery of the effects within at least three weeks. In addition, histopathology did not reveal any liver toxicity. In cynomolgus, the biochemical parameters for the animals of the liposome-treated group and for the test item-treated animals in comparison to the control animals were considered to lie within the limits of normal biological variability. Increased CK noted for individual animals of groups 4, 5, or 6 in comparison to the control animals on test days 9, 16, or 23 was mainly attributable to the CK-MM fraction and considered stress-related. Mild elevation of liver parameters in mice may be a reaction by immunomodulatory effects that may be triggered by the phagocytosis of RNA(LIP) by liver target cells, such as Kupffer cells. In contrast to the effects observed in the spleen of mice (lymphoid hyperplasia) this does not lead to a recruitment of leukocytes to the liver suggesting that desired pharmacological effects such as TLR activation and lymphocyte trafficking are limited to the lymphoid organ. Complement activation has been reported previously for liposome formulated substances. For RNA(LIP) vaccination slightly elevated C5a levels were observed in female mice, but this was regarded as event with low biological relevance. In addition, mice are not considered as a good model for extrapolation of complement effects to humans.

Overall, the immunological responses seen in all three RNA(LIP) repeated-dose toxicity studies (LPT No. 28864, 30283, and 30586) suggest a comprehensive picture from increase of spleen weight, cytokine/chemokine activation, and lymphocyte trafficking. This reflects induction of the intended pharmacological events and underlines the relevance of the mouse as the correct test model for toxicity studies. In the bridging study LPT No. 30583 evaluating the toxicity of the pH-adapted L2 Liposomal formulation no noteworthy differences were observed between the two liposome formulations. In this trial L4 pH-adapted liposomes which are half concentrated L2 Liposomes will be applied. No further bridging studies were planned as the same characteristics as for L2 Liposomes are given.

### Example 3: Induction of Antigen-Specific T Cells in the Spleen by KLK2-, KLK3-, ACPP-, NKX3-1- and HOXB13-Coding RNA

At day 1, day 8, day 15, and day 22 A2/DR1 mice were immunized with 200 μL of a 0.15 mg/mL RNA(Lip) solution which corresponds to 30 μg RNA(Lip).

Splenocytes were obtained from the immunized A2/DR1 mice five days after the final of four RNA(Lip) injections, and in vivo induction ofantigen-specificTcells was determined by ELISPOT analysis. To test for the immunogenicity, isolated splenocytes were re-stimulated with a peptide pool (15 mers, overlapping by 11 amino acids) spanning the respective human proteins, KLK2, KLK3 (PSA), ACPP (PAP), NKX3-1 or HOXB13. Results are shown in FIG. 20. IFN-γ+ spot count induced by KLK2-, ACPP-, and HOXB13-coding RNA(Lip) was statistically higher compared to restimulation with the control peptide CMV pp65 according to an unpaired t test (P=0.0056; P>0.0001; P=0.0095). In all immunized mice, restimulation of splenocytes with control P2/P16/P17 peptides also resulted in high spot counts indicating successful immunization. On all ELISPOT plates, the negative control medium alone induced only a minimal spot count regardless of the animal the splenocytes had been derived from.

1. A composition or medical preparation comprising at least one RNA, wherein the at least one RNA encodes the following amino acid sequences:
(i) an amino acid sequence comprising Kallikrein-2 (KLK2), an immunogenic variant thereof, or an immunogenic fragment of the KLK2 or the immunogenic variant thereof,
(ii) an amino acid sequence comprising Prostate Specific Antigen (PSA), an immunogenic variant thereof, or an immunogenic fragment of the PSA or the immunogenic variant thereof;
(iii) an amino acid sequence comprising Prostatic Acid Phosphatase (PAP), an immunogenic variant thereof, or an immunogenic fragment of the PAP or the immunogenic variant thereof;
(iv) an amino acid sequence comprising Homeobox B13 (HOXB13), an immunogenic variant thereof, or an immunogenic fragment of the HOXB13 or the immunogenic variant thereof; and
(v) an amino acid sequence comprising NK3 Homeobox 1 (NKX3-1), an immunogenic variant thereof, or an immunogenic fragment of the NKX3-1 or the immunogenic variant thereof.

2. The composition or medical preparation of claim 1, wherein each of the amino acid sequences under (i), (ii), (iii), (iv), or (v) is encoded by a separate RNA.

3. The composition or medical preparation of claim 1, wherein
the RNA encoding the amino acid sequence under (i) comprises the nucleotide sequence of SEQ ID NO: 3 or 4, or a nucleotide sequence having at least 99%, 98%, 97%, 96%, 95%, 90%, 85%, or 80% identity to the nucleotide sequence of SEQ ID NO: 3 or 4; and/or
the amino acid sequence under (i) comprises the amino acid sequence of SEQ ID NO: 1 or 2, or an amino acid sequence having at least 99%, 98%, 97%, 96%, 95%, 90%, 85%, or 80% identity to the amino acid sequence of SEQ ID NO: 1 or 2; and/or
the RNA encoding the amino acid sequence under (ii) comprises the nucleotide sequence of SEQ ID NO: 7 or 8, or a nucleotide sequence having at least 99%, 98%, 97%, 96%, 95%, 90%, 85%, or 80% identity to the nucleotide sequence of SEQ ID NO: 7 or 8; and/or
the amino acid sequence under (ii) comprises the amino acid sequence of SEQ ID NO: 5 or 6, or an amino acid sequence having at least 99%, 98%, 97%, 96%, 95%, 90%, 85%, or 80% identity to the amino acid sequence of SEQ ID NO: 5 or 6; and/or
the RNA encoding the amino acid sequence under (iii) comprises the nucleotide sequence of SEQ ID NO: 11 or 12, or a nucleotide sequence having at least 99%, 98%, 97%, 96%, 95%, 90%, 85%, or 80% identity to the nucleotide sequence of SEQ ID NO: 11 or 12; and/or
the amino acid sequence under (iii) comprises the amino acid sequence of SEQ ID NO: 9 or 10, or an amino acid sequence having at least 99%, 98%, 97%, 96%, 95%, 90%, 85%, or 80% identity to the amino acid sequence of SEQ ID NO: 9 or 10; and/or
the RNA encoding the amino acid sequence under (iv) comprises the nucleotide sequence of SEQ ID NO: 15 or 16, or a nucleotide sequence having at least 99%, 98%, 97%, 96%, 95%, 90%, 85%, or 80% identity to the nucleotide sequence of SEQ ID NO: 15 or 16; and/or
the amino acid sequence under (iv) comprises the amino acid sequence of SEQ ID NO: 13 or 14, or an amino acid sequence having at least 99%, 98%, 97%, 96%, 95%, 90%, 85%, or 80% identity to the amino acid sequence of SEQ ID NO: 13 or 14; and/or
the RNA encoding the amino acid sequence under (v) comprises the nucleotide sequence of SEQ ID NO: 19 or 20, or a nucleotide sequence having at least 99%, 98%, 97%, 96%, 95%, 90%, 85%, or 80% identity to the nucleotide sequence of SEQ ID NO: 19 or 20; and/or
the amino acid sequence under (v) comprises the amino acid sequence of SEQ ID NO: 17 or 18, or an amino acid sequence having at least 99%, 98%, 97%, 96%, 95%, 90%, 85%, or 80% identity to the amino acid sequence of SEQ ID NO: 17 or 18.

4-7. (canceled)

8. The composition or medical preparation of claim 1, wherein at least one of or each of the amino acid sequences under (i), (ii), (iii), (iv), or (v) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

9. (canceled)

10. The composition or medical preparation of claim 1, wherein at least one or each RNA comprises the 5′ cap m₂^{7,2′-O}Gppₛp(5′)G.

11. (canceled)

12. The composition or medical preparation of claim 1, wherein at least one or each RNA comprises a 5′ UTR comprising the nucleotide sequence of SEQ ID NO: 21, or a nucleotide sequence having at least 99%, 98%, 97%, 96%, 95%, 90%, 85%, or 80% identity to the nucleotide sequence of SEQ ID NO: 21.

13. (canceled)

14. The composition or medical preparation of claim 1, wherein at least one or each amino acid sequence under (i), (ii), (iii), (iv), or (v) comprises an amino acid sequence enhancing antigen processing and/or presentation.

15. (canceled)

16. The composition or medical preparation of claim 14, wherein the amino acid sequence enhancing antigen processing and/or presentation comprises an amino acid sequence corresponding to the transmembrane and cytoplasmic domain of a MHC molecule, preferably a MHC class I molecule.

17. The composition or medical preparation of claim 14, wherein
(i) the RNA encoding the amino acid sequence enhancing antigen processing and/or presentation comprises the nucleotide sequence of SEQ ID NO: 25, or a nucleotide sequence having at least 99%, 98%, 97%, 96%, 95%, 90%, 85%, or 80% identity to the nucleotide sequence of SEQ ID NO: 25; and/or
(ii) the amino acid sequence enhancing antigen processing and/or presentation comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24.

18. The composition or medical preparation of claim 1, wherein at least one of or each amino acid sequence under (i), (ii), (iii), (iv), or (v) comprises an amino acid sequence which breaks immunological tolerance.

19. (canceled)

20. The composition or medical preparation of claim 18, wherein the amino acid sequence which breaks immunological tolerance comprises helper epitopes, preferably tetanus toxoid-derived helper epitopes.

21. The composition or medical preparation of claim 18, wherein
(i) the RNA encoding the amino acid sequence which breaks immunological tolerance comprises the nucleotide sequence of SEQ ID NO: 27, or a nucleotide sequence having at least 99%, 98%, 97%, 96%, 95%, 90%, 85%, or 80% identity to the nucleotide sequence of SEQ ID NO: 27; and/or
(ii) the amino acid sequence which breaks immunological tolerance comprises the amino acid sequence of SEQ ID NO: 26, or an amino acid sequence having at least 99%, 98%, 97%, 96%, 95%, 90%, 85%, or 80% identity to the amino acid sequence of SEQ ID NO: 26.

22. The composition or medical preparation of claim 1, wherein at least one RNA comprises a 3′ UTR comprising the nucleotide sequence of SEQ ID NO: 28, or a nucleotide sequence having at least 99%, 98%, 97%, 96%, 95%, 90%, 85%, or 80% identity to the nucleotide sequence of SEQ ID NO: 28.

23. (canceled)

24. The composition or medical preparation of claim 1, wherein at least one or each RNA comprises a poly-A sequence.

25. (canceled)

26. The composition or medical preparation of claim 24, wherein the poly-A sequence comprises at least 100 nucleotides.

27. The composition or medical preparation of claim 24, wherein the poly-A sequence comprises or consists of the nucleotide sequence of SEQ ID NO: 29.

28-32. (canceled)

33. The composition or medical preparation of claim 1, which is a pharmaceutical composition.

34. The composition or medical preparation of claim 33, wherein the pharmaceutical composition further comprises one or more pharmaceutically acceptable carriers, diluents and/or excipients.

35. The composition or medical preparation of claim 1, wherein the medical preparation is a kit.

36-47. (canceled)

48. A method of treating prostate cancer in a subject comprising administering at least one RNA to the subject, wherein the at least one RNA encodes the following amino acid sequences:
(i) an amino acid sequence comprising Kallikrein-2 (KLK2), an immunogenic variant thereof, or an immunogenic fragment of the KLK2 or the immunogenic variant thereof,
(ii) an amino acid sequence comprising Prostate Specific Antigen (PSA), an immunogenic variant thereof, or an immunogenic fragment of the PSA or the immunogenic variant thereof;
(iii) an amino acid sequence comprising Prostatic Acid Phosphatase (PAP), an immunogenic variant thereof, or an immunogenic fragment of the PAP or the immunogenic variant thereof;
(iv) an amino acid sequence comprising Homeobox B13 (HOXB13), an immunogenic variant thereof, or an immunogenic fragment of the HOXB13 or the immunogenic variant thereof; and
(v) an amino acid sequence comprising NK3 Homeobox 1 (NKX3-1), an immunogenic variant thereof, or an immunogenic fragment of the NKX3-1 or the immunogenic variant thereof.

49. The method of claim 48, wherein each of the amino acid sequences under (i), (ii), (iii), (iv), or (v) is encoded by a separate RNA.

50. The method of claim 48, wherein
the RNA encoding the amino acid sequence under (i) comprises the nucleotide sequence of SEQ ID NO: 3 or 4, or a nucleotide sequence having at least 99%, 98%, 97%, 96%, 95%, 90%, 85%, or 80% identity to the nucleotide sequence of SEQ ID NO: 3 or 4; and/or
the amino acid sequence under (i) comprises the amino acid sequence of SEQ ID NO: 1 or 2, or an amino acid sequence having at least 99%, 98%, 97%, 96%, 95%, 90%, 85%, or 80% identity to the amino acid sequence of SEQ ID NO: 1 or 2; and/or
the RNA encoding the amino acid sequence under (ii) comprises the nucleotide sequence of SEQ ID NO: 7 or 8, or a nucleotide sequence having at least 99%, 98%, 97%, 96%, 95%, 90%, 85%, or 80% identity to the nucleotide sequence of SEQ ID NO: 7 or 8; and/or
the amino acid sequence under (ii) comprises the amino acid sequence of SEQ ID NO: 5 or 6, or an amino acid sequence having at least 99%, 98%, 97%, 96%, 95%, 90%, 85%, or 80% identity to the amino acid sequence of SEQ ID NO: 5 or 6; and/or
the RNA encoding the amino acid sequence under (iii) comprises the nucleotide sequence of SEQ ID NO: 11 or 12, or a nucleotide sequence having at least 99%, 98%, 97%, 96%, 95%, 90%, 85%, or 80% identity to the nucleotide sequence of SEQ ID NO: 11 or 12; and/or
the amino acid sequence under (iii) comprises the amino acid sequence of SEQ ID NO: 9 or 10, or an amino acid sequence having at least 99%, 98%, 97%, 96%, 95%, 90%, 85%, or 80% identity to the amino acid sequence of SEQ ID NO: 9 or 10; and/or
the RNA encoding the amino acid sequence under (iv) comprises the nucleotide sequence of SEQ ID NO: 15 or 16, or a nucleotide sequence having at least 99%, 98%, 97%, 96%, 95%, 90%, 85%, or 80% identity to the nucleotide sequence of SEQ ID NO: 15 or 16; and/or
the amino acid sequence under (iv) comprises the amino acid sequence of SEQ ID NO: 13 or 14, or an amino acid sequence having at least 99%, 98%, 97%, 96%, 95%, 90%, 85%, or 80% identity to the amino acid sequence of SEQ ID NO: 13 or 14; and/or
the RNA encoding the amino acid sequence under (v) comprises the nucleotide sequence of SEQ ID NO: 19 or 20, or a nucleotide sequence having at least 99%, 98%, 97%, 96%, 95%, 90%, 85%, or 80% identity to the nucleotide sequence of SEQ ID NO: 19 or 20; and/or
the amino acid sequence under (v) comprises the amino acid sequence of SEQ ID NO: 17 or 18, or an amino acid sequence having at least 99%, 98%, 97%, 96%, 95%, 90%, 85%, or 80% identity to the amino acid sequence of SEQ ID NO: 17 or 18.

51-74. (canceled)

75. The method of claim 48, wherein the RNA is administered by injection.

76. The method of claim 48, wherein the RNA is administered by intravenous administration.

77. The method of claim 48, wherein the RNA is formulated as lipoplex particles.

78. The method of claim 77, wherein the RNA lipoplex particles are obtainable by mixing the RNA with liposomes.

79. The method of claim 48, wherein the subject is a human.

80. The method of claim 48, which further comprises administering a further therapy.

81. The method of claim 80, wherein the further therapy comprises one or more selected from the group consisting of: (i) surgery to excise, resect, or debulk a tumor, (ii) radiotherapy, and (iii) chemotherapy.

82. The method of claim 80, wherein the further therapy comprises administering a further therapeutic agent.

83. The method of claim 82, wherein the further therapeutic agent comprises an anti-cancer therapeutic agent.

84. The method of claim 82, wherein the further therapeutic agent is a checkpoint modulator.

85. The method of claim 84, wherein the checkpoint modulator is an anti-PD1 antibody, an anti-CTLA-4 antibody, or a combination of an anti-PD1 antibody and an anti-CTLA-4 antibody.
